(19) United States
(12) Patent Application Publication
(10) Pub. No.: US 2015/0197497 A1
(43) Pub. Date: Jul. 16, 2015

(54) SELECTIVE INHIBITORS OF HISTONE DEACETYLASE ISOFORM 6 AND METHODS THEREOF

(71) Applicants: Chandima Abeywickrama, Wayne, NJ (US); James E. Bradner, Weston, MA (US); Shashikanth Ponnala, Jersey City, NJ (US)

(72) Inventors: Chandima Abeywickrama, Wayne, NJ (US); James E. Bradner, Weston, MA (US); Shashikanth Ponnala, Jersey City, NJ (US)

(73) Assignee: DANA-FARBER CANCER INSTITUTE, INC., Boston, MA (US)

(21) Appl. No.: 14/129,108

(22) PCT Filed: Jun. 25, 2012
(86) PCT No.: PCT/US12/44087

§ 371 Date: Mar. 17, 2015

Related U.S. Application Data
(60) Provisional application No. 61/500,785, filed on Jun. 24, 2011.

(51) Int. Cl. C07D 249/04; C07D 235/08; C07D 235/26; C07D 235/10

(52) U.S. Cl. CPC C07D 249/04; C07D 235/10; C07D 235/08; C07D 235/26

(57) ABSTRACT

The described invention provides histone deacetylase (HDAC) inhibitor compounds with substituted benzimidazole, benzimidazolone and benzotriazole heterocycles showing selective inhibition of histone deacetylase isoform HDAC6. The described invention further provides methods of making such compounds and methods of inhibiting HDAC, treating HDAC-associated diseases, including cell proliferative disorders, such as cancer, autoimmune or inflammatory diseases and neurodegenerative diseases.

### CROSS-REFERENCE TO RELATED APPLICATIONS

This application is a U.S. national stage entry of PCT/U.S. Ser. No. 12/044,087 (filed Jun. 25, 2012), which claims the benefit of priority to U.S. Provisional application No. 61/500,785, filed Jun. 24, 2011. The contents of each of these applications are incorporated herein by reference in their entirety.

### STATEMENT OF GOVERNMENT FUNDING

This invention was made with government support awarded by the National Institute of Health. The government has certain rights in the invention.

### FIELD OF THE INVENTION

The described invention relates to compounds of Formula I, derivatives, prodrugs and pharmaceutically acceptable salts, compositions and kits comprising such compounds, methods for making, and methods of use in treating histone deacetylase-associated disorders.

### BACKGROUND

In eukaryotic cells, DNA is packaged into a higher order compact complex known as chromatin by virtue of the tight binding of highly conserved positively charged histone proteins with negatively charged phosphate groups of nuclear DNA. The fundamental unit of nuclear chromatin is a nucleosome. Each nucleosome comprises a stretch of about 200 base pairs of DNA wrapped in two loops around a central core containing an octamer of two copies each of four histone proteins, H2A, H2B, H3 and H4. Individual nucleosomes are connected by short stretches of DNA, known as linker DNA to form a “beads on a string” structure. The linker DNA is variable in length ranging from about 8 to 114 base pairs and remains in tight association with a fifth histone, H1. The beaded string nucleosome structure is further organized into a 30 nm helical solenoid fiber comprising about six nucleosomes per turn. Solenoid fibers are further packaged to form chromosomes by extensive looping of solenoid fibers.

Histone proteins undergo posttranslational modifications of various types, including, but not limited to, methylation of lysine and arginine groups, acetylation of lysine groups, phosphorylation of serine groups and ubiquitination of lysine groups. (Reviewed in Kouzarides, T. et al., Cell, 128:693-705 (2007)). These modifications play an important role in the regulation of gene transcription by controlling the recruitment of non-histone proteins, such as transcription factors, to specific DNA sequences. One of these modifications, namely reversible acetylation of histones, neutralizes the positive charge of lysine residues, thereby lowering the binding affinities of histones to DNA, resulting in destabilization, and subsequent loosening of chromatin structure, thereby increasing the accessibility of specific DNA sequences to transcription factors, facilitating transcription. Histone acetylation levels are maintained by a delicate balance between activities of two enzymes: histone acetyl transferases (HATs) and histone deacetylases (HDACs).

The HDAC enzyme family constitutes a family of 18 genes that can be grouped into four subclasses; classes I-IV, based on their homology to respective yeast orthologs. HDACs belonging to classes I, II and IV, which comprise 11 members, namely HDAC isoforms 1-11, commonly referred to as the classical HDACs, are metal-dependent hydrolases. HDACs of class III, which comprise 7 members, known as sirtuins, namely Sirt 1-7, are NAD+-dependent hydrolases. Class I HDACs are nuclear proteins with ubiquitous tissue expression. Class II and IV HDACs are found in both the nucleus and cytoplasm and exhibit tissue-specific expression. The Class II HDAC family is further subdivided into subclasses IIA and IIB. Class IIA comprises isoforms HDAC4, HDAC5, HDAC7 and HDAC9 while Class IIB comprises isoforms HDAC6 and HDAC10. HDAC6 contains two tandem deacetylase domains and a C-terminal zinc finger domain. HDAC10 is structurally related to HDAC6 but has one additional catalytic domain. Table 1 represents the cellular location and tissue expression of classical HDACs (adapted from Witt, O. et al., Cancer Lett., 277:8-21 (2008)).

TABLE 1
Classical HDACs, Cellular Location and Tissue Expression

| Class | Isoform | Cellular Location | Tissue Expression |
|---|---|---|---|
| Class I | HDAC1 | Nuclear | Ubiquitous |
| | HDAC2 | Nuclear | Ubiquitous |
| | HDAC3 | Nuclear | Ubiquitous |
| | HDAC8 | Nuclear/cytoplasmic | Ubiquitous |
| Class IIA | HDAC4 | Nuclear/cytoplasmic | Heart, smooth |
| | | | muscles, brain |
| | HDAC5 | Nuclear/cytoplasmic | Heart, smooth |
| | | | muscle, brain |
| | HDAC7 | Nuclear/cytoplasmic | Heart, placenta, |
| | | | pancreas, smooth muscle |
| | HDAC9 | Nuclear/cytoplasmic | Smooth muscle, |
| | | | brain |
| Class IIB | HDAC6 | Cytoplasmic | Kidney, liver, |
| | | | heart, pancreas |
| | HDAC10 | Cytoplasmic | Spleen, kidney, liver |
| Class IV | HDAC11 | Nuclear/cytoplasmic | Heart, smooth muscle, |
| | | | kidney, brain |

HDACs play a significant role in both normal and aberrant cell proliferation and differentiation. HDACs have been associated with a number of diseased states involving proliferation, including, but not limited to, cell proliferative diseases and conditions, such as various forms of cancer. (Reviewed in Witt, O. et al., Cancer Lett., 277:8-21 (2008); and Portella A. et al., Nat. Biotechnol., 28:1057-1068 (2010)). Class I and II HDACs have been identified as attractive targets for anticancer therapy. In particular, distinct class I and class II HDAC proteins are overexpressed in some cancers, including ovarian (HDAC1-3), gastric (HDAC2), and lung cancers (HDAC1 and 3), among others. In addition, a possible correlation between HDAC8 and acute myeloid leukemia (AML) has been suggested. With respect to class II HDAC proteins, aberrant expression of HDAC6 is induced in some breast cancer cells. Based on their clinical effects, HDAC inhibitors have been identified that suppress tumor cell proliferation, induce cell differentiation, and upregulate crucial genes associated with anti-cancer effects. HDACs have also been implicated in various types of cancers (Bali P, et al, “Inhibition of histone deacetylase 6 acetylates and disrupts the chaperone function of heat shock protein 90: A novel basis for antileukemia activity of histone deacetylase inhibitors,” J. Biol. Chem., 2005 280:26729-26734; Santo L. et al., “Preclinical activity, pharmacodynamic and pharmacokinetic properties of a selective HDAC6 inhibitor, ACY-1215, in combination with bortezomib in multiple myeloma,” Blood, 2012, 119(11): 2579-89), autoimmune or inflammatory diseases (Shuttleworth, S. J., et al., Curr. Drug Targets, 11:1430-1438 (2010)), cognitive and neurodegenerative diseases (Fischer, A., et al., Trends Pharmacol. Sci., 31:605-617 (2010); Chuang, D.-M., et al., Trends Neurosci. 32:591-601 (2009)), fibrotic diseases (Pang, M. et al., J. Pharmacol. Exp. Ther., 335:266-272 (2010)), protozoal diseases (see, e.g., U.S. Pat. No. 5,922,837), and viral diseases (Margolis, D. M. et al., Curr. Opin. HIV AIDS, 6:25-29 (2011)).

A large number of HDAC inhibitors (HDACi) have been reported. These can be structurally classified into short chain fatty acids, including not limited to butyrate and valproate, depsipeptides, including but not limited to apicidin, FK228, etc., and inhibitors, such as inhibitors of class I and class II HDAC enzymes, with a general structure characterized by a metal-binding motif (usually zinc-binding), a linker, and a capping group, also known as a surface recognition motif (Reviewed in Paris, M. et al., J. Med. Chem., 51:1505-1529 (2008)). The class I and class II inhibitors can be further grouped into two broad categories depending on the metal binding moiety: hydroxamic acid derivatives and non-hydroxamic acid derivatives. Hydroxamic acid derivatives can be further classified into subclasses depending on the nature of the linker: hydroxamic acid derivatives with linear linkers, hydroxamic acid derivatives with cinnamyl and aromatic linkers and hydroxamic acid derivatives with heteroaromatic linkers. Non-hydroxamic acid derivatives can be further classified into three subclasses depending on the nature of the metal binding group: thiols and thiol derivatives, benzamides and ketones. For example, hydroxamic acid derivatives with linear linkers include, but are not limited to, trichostatin A (TSA), suberoylanilide hydroxamic acid (SAHA), CRA-A, etc. For example, a hydroxamic acid derivative with aromatic linker includes, but is not limited to, MS-244. For example, a benzamide derivative includes, but is not limited to, MS 27-275. Exemplary inhibitors of each class as disclosed in the art have been reviewed in Paris et al., Id.

HDAC6 is primarily cytoplasmic and regulates acetylation of many cytoplasmic proteins, including but not limited to α-tubulin and heat shock protein 90 (HSP90), as described in Bali, P. et al., “Inhibition of histone deacetylase 6 acetylates and disrupts the chaperone function of heat shock protein 90: a novel basis for antileukemia activity of histone deacetylase inhibitors,” J. Biol. Chem., 2005, 280: 26729-26734; Grozinger, C. M. et al., “Three proteins define a class of a human histone deacetylases related to yeast Hdalp,” Proc. Natl. Acad. Sci. U.S.A., 1999, 96: 4868-4873; Hubert, C. et al., “HDAC6 is a microtubule associated deacetylase,” Nature, 2002, 417: 455-458; Kovacs, J. J. et al., “HDAC6 regulates Hsp90 acetylation and chaperone-dependent activation of glucocorticoid receptor,” Mol. Cell., 2005, 18: 601-607; Valenzuela-Fernandez, A. et al., “HDAC6: a key regulator of cytoskeleton, cell migration and cell-cell interactions,” Trends Cell Biol., 2008, 18: 291-297; and de Zoeten, E. F. et al., “Histone deacetylase 6 and heat shock protein 90 control the functions of Foxp3+T-regulatory cells,” Mol. Cell. Biol., 2011, 31(10): 2066-2078.

Further exemplary HDAC inhibitors have been described, such as: bicyclic hydroxamic acid derivatives (see, e.g., WO 2003/066579); substituted piperazinylpyrimidinylhydroxamic acid derivatives (see, e.g., WO 2003/075929, WO 2003/076395, WO 2003/076400, WO 2003/076401, WO 2003/076421, WO 2003/076422, WO 2003/076430, WO 2003/076438, WO 2003/076422); carbamic acid derivatives with piperazine linkers (see, e.g., WO 2003/082288); substituted piperazinyl phenyl benzamide derivatives (see, e.g., WO 2003/087057); benzamides (see, e.g., WO 2003/092686); derivatives containing an alkyl linker between the aryl group and the hydroxamate (see, e.g., WO 2004/009536); (hetero)arylalkenyl substituted bicyclic hydroxamates (see, e.g., WO 2004/013130); arylene-carboxylic acid (2-amino-phenyl)-amide derivatives (see, e.g., WO 2004/056748); N-hydroxy-benzamide derivatives with anti-inflammatory and antitumor activity (see, e.g., WO 2004/063146); substituted aryl hydroxamate derivatives (see, e.g., WO 2004/063169); mono-acylated 0-phenylendiamines derivatives (see, e.g., WO 2004/069803); diaminophenylene derivatives (see, e.g., WO 2004/069823); benzamide derivatives (see, e.g., WO 2004/071400); indoles, benzimidazoles and naphhimidazoles (see, e.g., WO 2004/072047); hydroxamates linked to non-aromatic heterocyclic ring systems (see, e.g., WO2004/08638); oxime derivatives (see, e.g., WO 2004/087693); hydroxamate derivatives (see, e.g., WO 2004/092115); benzimidazoles (see, e.g., WO 2005/028447); benzamides (see, e.g., WO 2005/030704 and WO 2005/030705); acylurea connected and sulfonylurea connected hydroxamates (see, e.g., WO 2005/040101); biaryl linked hydroxamates (see, e.g., WO 2005/040161); thiazolyl hydroxamic acids and thiadiazolyl hydroxamic acids (see, e.g., WO 2005/075469); heteropentacyclic hydroxamic acids (see, e.g., WO 2005/086898); alkenylbenzamides (see, e.g., WO 2005/092899), etc.

The majority of the small molecule HDAC inhibitors in use or being evaluated in clinical trials inhibit all HDAC isoforms nonspecifically. Such non-specific inhibitors are known as pan-inhibitors. (Bradner, J. E. et al., 2010, Nat. Chem. Biol., 6:238-243). For example, SAHA and TSA are canonical pan-inhibitors, influencing the activity of HDAC1-9 isoforms with roughly equivalent potency. Only two of the eleven HDAC isoforms have been tested for isoform selectivity (e.g. trapoxin and tubacin). (Bieliauskas A. V. et al., Chem. Soc. Rev., 37:1402-1413 (2008)).

Non-selective HDAC inhibitors have been associated with toxicity and side effects, such as nausea and vomiting. SAHA (vorinostat; Merck Research Laboratories) and FK-228 (romidepsin, istodax; Gloucester Pharmaceuticals) are two pharmaceutical HDAC inhibitor drugs approved for use in humans. These drugs are used for the treatment of advanced cutaneous T-cell lymphoma (CTCL). The most common drug-related adverse reactions with SAHA include pulmonary embolism, deep vein thrombosis and anemia. FK-228 is known to cause nausea, vomiting, diarrhea, constipation, anemia, ECG T-wave changes, neutropenia, and lymphopenia. Despite the therapeutic advantage of isoform-selective HDAC inhibitors, design of such inhibitors has been challenging due to the high sequence similarity within the active sites of the isoforms. (Bradner, J. E. et al., Nat. Chem. Biol., 6:238-243 (2010)).

The described invention provides inhibitors that are structurally distinct from known HDAC inhibitors, HDAC6 show higher activity to all HDAC isoforms as compared to known HDAC inhibitors and three to four orders of magnitude higher activity to HDAC6 as compared to other HDAC isoforms, thereby showing selectivity toward HDAC6 isoform.

### SUMMARY

According to one aspect, the present invention provides a compound of Formula I:

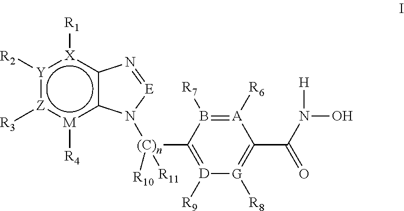

or a pharmaceutically acceptable salt thereof, wherein: each of X, Y, Z and M is independently C or N; each of R₁, R₂, R₃ and R₄ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₁, R₂, R₃ and R₄ is H or a substituent when X, Y, Z and M is carbon; E is C—R₅, or N; R₅ is H, OH, NH₂, amino optionally substituted by alkyl or aryl, CN, F, Cl, Br, I, C₁-C₆ perfluoroalkyl, O-alkyl, O-aryl, O-heteroaryl, NO₂, cycloalkyl, aryl, acyl, optionally substituted C₁-C₆ alkyl, C₂-C₆ alkene, or C₂-C₆ alkyne, wherein when R₅ is OH, the compound exists as a keto tautomer, as an enol tautomer or as a mixture of keto-enol tautomers; each of A, B, D, and G is independently C or N; each of R₆, R₇, R₈, and R₉ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₆, R₇, R₈ and R₉ is H or a substituent when A, B, D and G is carbon; each of R₁₀ and R₁₁ is independently H, alkyl, or aryl, wherein (C)ₙ optionally is a chiral center, wherein (C)ₙ can exist as both R and S enantiomers, with the proviso that when R₁₀ is H, R₁₁ is alkyl or aryl; and when R₁₁ is H, R₁₀ is alkyl or aryl; and n is 0, 1, 2, 3, 4, 5, 6, 7, 8, 9 or 10, wherein the compound is a histone deacetylatase (HDAC) inhibitor, and wherein the HDAC inhibitor inhibits histone deacetylating activity of at least one HDAC isoform selected from the group consisting of HDAC1, HDAC2, HDAC3, HDAC4, HDAC5, HDAC6, HDAC7, HDAC8, HDAC9, and a combination thereof. According to one embodiment, the compound of formula I is a compound of Formula Ia:

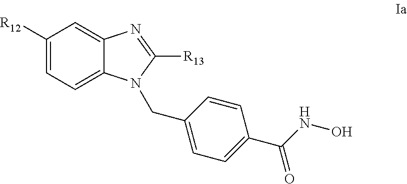

or a pharmaceutically acceptable salt thereof, wherein: R₁₂ is selected from the group consisting of H, alkyl, F, Cl, Br, I, and O-alkyl; and R₁₃ is selected from the group consisting of H and C₁-C₆ perfluoroalkyl. According to another embodiment, the compound of Formula I is a compound of Formula Ib:

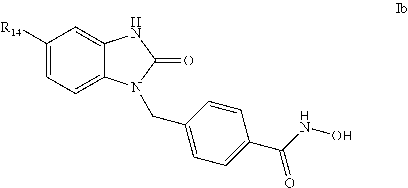

or a pharmaceutically acceptable salt thereof, wherein: R₁₄ is selected from the group consisting of H, alkyl, F, Cl, Br, I, O-alkyl, and C₁-C₆ perfluoroalkyl. According to another embodiment, the compound of Formula I is a compound of Formula Ic:

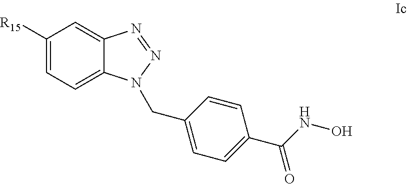

or a pharmaceutically acceptable salt thereof, wherein: R₁₅ is selected from the group consisting of H, alkyl, F, Cl, Br, I, and O-alkyl. According to another embodiment, the HDAC inhibitor inhibits the histone deacetylating activity of at least one HDAC isoform with an inhibition activity (IC₅₀) from about 0.005 μM to about 2.76 μM. According to another embodiment, the HDAC inhibitor inhibits the histone deacetylating activity of HDAC6 with an inhibition activity (IC₅₀) from about 0.000001 μM to about 0.001 μM. According to another embodiment, the HDAC inhibitor is selective toward HDAC6. According to another embodiment, a ratio of the inhibitory activity (IC₅₀) of the HDAC inhibitor obtained in the presence of an HDAC isoform selected from the group consisting of HDAC1, HDAC2, HDAC3, HDAC4, HDAC5, HDAC7, HDAC8, and HDAC9, to the inhibition activity (IC₅₀) value of the HDAC inhibitor selective toward HDAC6 obtained in vitro in the presence of HDAC6 (in vitro selectivity value) has a value of at least 100. According to another embodiment, a ratio of the inhibitory activity (IC₅₀) of the HDAC inhibitor obtained in the presence of an HDAC isoform selected from the group consisting of HDAC1, HDAC2, HDAC3, HDAC4, HDAC5, HDAC7, HDAC8, and HDAC9, to the inhibition activity (IC₅₀) value of the HDAC inhibitor selective toward HDAC6 obtained in vitro in the presence of HDAC6 (in vitro selectivity value) has a value of at least 30,000. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 2.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 50.0. According to another embodiment, the compound is selected from:

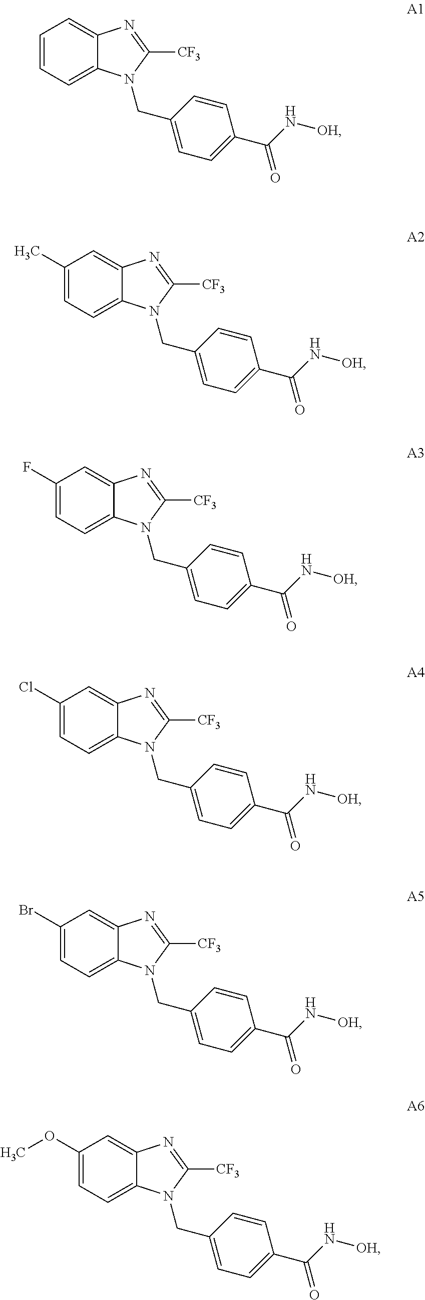 [SMILES: O=C(NO)c1ccc(Cn2c(C(F)(F)F)nc3ccccc32)cc1 ; Cc1ccc2c(c1)nc(C(F)(F)F)n2Cc1ccc(C(=O)NO)cc1 ; O=C(NO)c1ccc(Cn2c(C(F)(F)F)nc3cc(F)ccc32)cc1 ; O=C(NO)c1ccc(Cn2c(C(F)(F)F)nc3cc(Cl)ccc32)cc1 ; O=C(NO)c1ccc(Cn2c(C(F)(F)F)nc3cc(Br)ccc32)cc1 ; COc1ccc2c(c1)nc(C(F)(F)F)n2Cc1ccc(C(=O)NO)cc1]

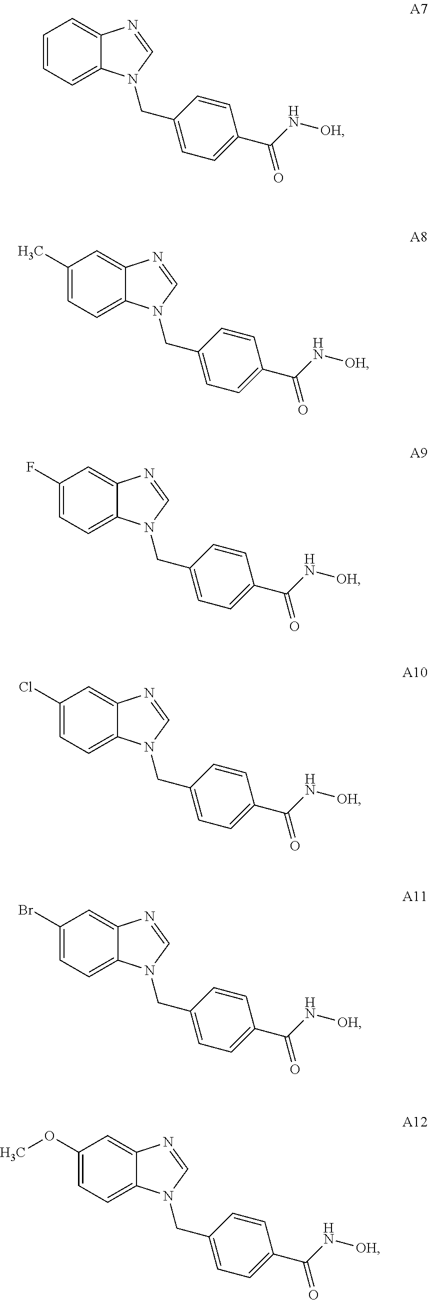 [SMILES: O=C(NO)c1ccc(Cn2cnc3ccccc32)cc1 ; Cc1ccc2c(c1)ncn2Cc1ccc(C(=O)NO)cc1 ; O=C(NO)c1ccc(Cn2cnc3cc(F)ccc32)cc1 ; O=C(NO)c1ccc(Cn2cnc3cc(Cl)ccc32)cc1 ; O=C(NO)c1ccc(Cn2cnc3cc(Br)ccc32)cc1 ; COc1ccc2c(c1)ncn2Cc1ccc(C(=O)NO)cc1]

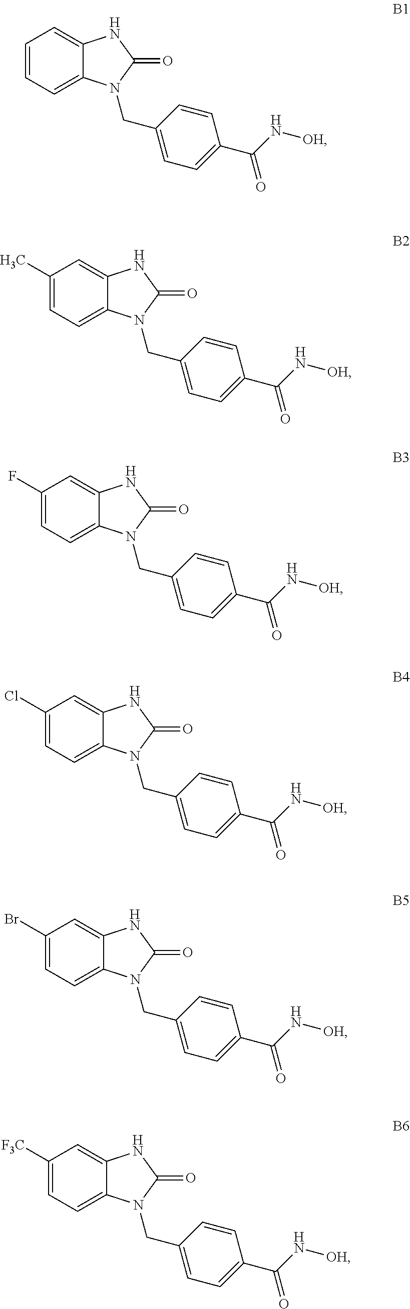 [SMILES: O=C(NO)c1ccc(Cn2c(=O)[nH]c3ccccc32)cc1 ; Cc1ccc2c(c1)[nH]c(=O)n2Cc1ccc(C(=O)NO)cc1 ; O=C(NO)c1ccc(Cn2c(=O)[nH]c3cc(F)ccc32)cc1 ; O=C(NO)c1ccc(Cn2c(=O)[nH]c3cc(Cl)ccc32)cc1 ; O=C(NO)c1ccc(Cn2c(=O)[nH]c3cc(Br)ccc32)cc1 ; O=C(NO)c1ccc(Cn2c(=O)[nH]c3cc(C(F)(F)F)ccc32)cc1]

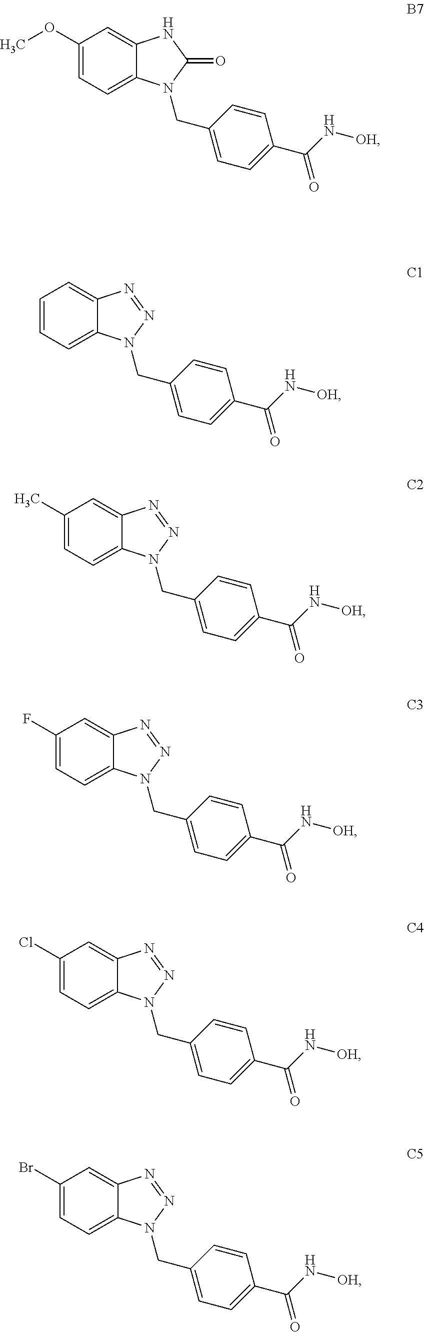 [SMILES: COc1ccc2c(c1)[nH]c(=O)n2Cc1ccc(C(=O)NO)cc1 ; O=C(NO)c1ccc(Cn2nnc3ccccc32)cc1 ; Cc1ccc2c(c1)nnn2Cc1ccc(C(=O)NO)cc1 ; O=C(NO)c1ccc(Cn2nnc3cc(F)ccc32)cc1 ; O=C(NO)c1ccc(Cn2nnc3cc(Cl)ccc32)cc1 ; O=C(NO)c1ccc(Cn2nnc3cc(Br)ccc32)cc1]

or a combination thereof. According to another embodiment, the compound is

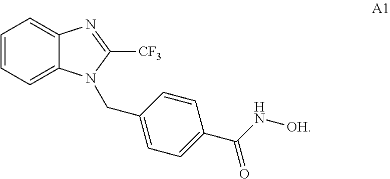 [SMILES: O=C(NO)c1ccc(Cn2c(C(F)(F)F)nc3ccccc32)cc1]

According to another embodiment, the compound is

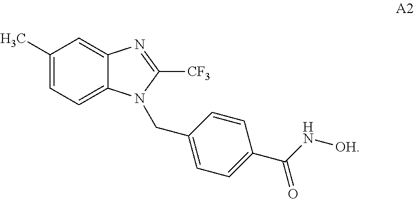 [SMILES: Cc1ccc2c(c1)nc(C(F)(F)F)n2Cc1ccc(C(=O)NO)cc1]

According to another embodiment, the compound is

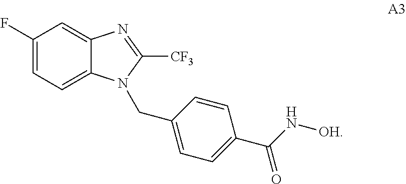 [SMILES: O=C(NO)c1ccc(Cn2c(C(F)(F)F)nc3cc(F)ccc32)cc1]

According to another embodiment, the compound is

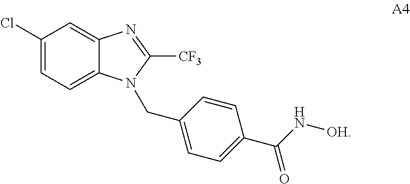 [SMILES: O=C(NO)c1ccc(Cn2c(C(F)(F)F)nc3cc(Cl)ccc32)cc1]

According to another embodiment, the compound is

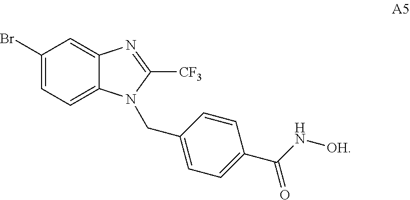 [SMILES: O=C(NO)c1ccc(Cn2c(C(F)(F)F)nc3cc(Br)ccc32)cc1]

According to another embodiment, the compound is

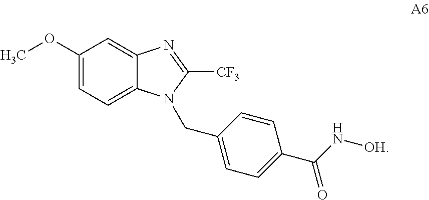 [SMILES: COc1ccc2c(c1)nc(C(F)(F)F)n2Cc1ccc(C(=O)NO)cc1]

According to another embodiment, the compound is

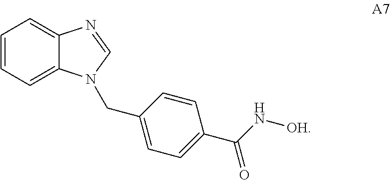 [SMILES: O=C(NO)c1ccc(Cn2cnc3ccccc32)cc1]

According to another embodiment, the compound is

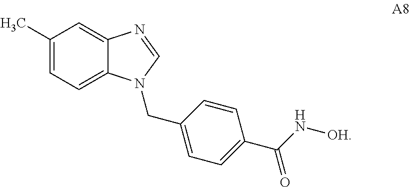 [SMILES: Cc1ccc2c(c1)ncn2Cc1ccc(C(=O)NO)cc1]

According to another embodiment, the compound is

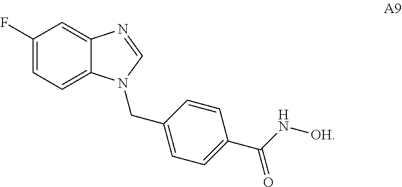 [SMILES: O=C(NO)c1ccc(Cn2cnc3cc(F)ccc32)cc1]

According to another embodiment, the compound is

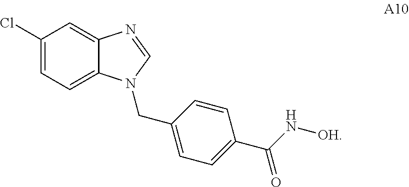 [SMILES: O=C(NO)c1ccc(Cn2cnc3cc(Cl)ccc32)cc1]

According to another embodiment, the compound is

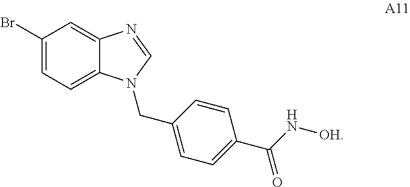 [SMILES: O=C(NO)c1ccc(Cn2cnc3cc(Br)ccc32)cc1]

According to another embodiment, the compound is

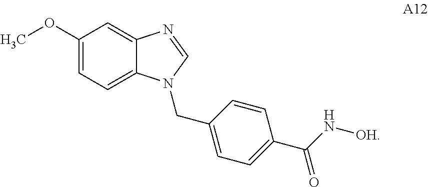 [SMILES: COc1ccc2c(c1)ncn2Cc1ccc(C(=O)NO)cc1]

According to another embodiment, the compound is

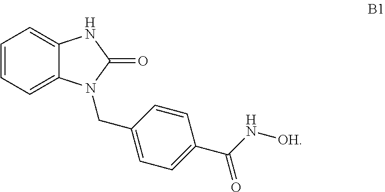 [SMILES: O=C(NO)c1ccc(Cn2c(=O)[nH]c3ccccc32)cc1]

According to another embodiment, the compound is

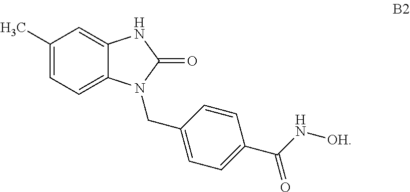 [SMILES: Cc1ccc2c(c1)[nH]c(=O)n2Cc1ccc(C(=O)NO)cc1]

According to another embodiment, the compound is

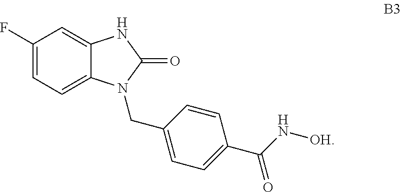 [SMILES: O=C(NO)c1ccc(Cn2c(=O)[nH]c3cc(F)ccc32)cc1]

According to another embodiment, the compound is

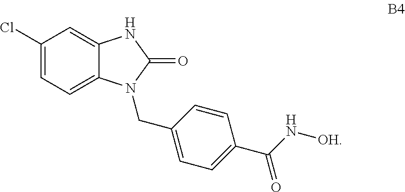 [SMILES: O=C(NO)c1ccc(Cn2c(=O)[nH]c3cc(Cl)ccc32)cc1]

According to another embodiment, the compound is

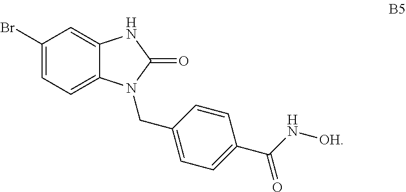 [SMILES: O=C(NO)c1ccc(Cn2c(=O)[nH]c3cc(Br)ccc32)cc1]

According to another embodiment, the compound is

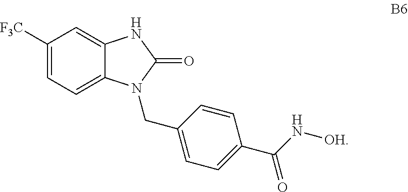 [SMILES: O=C(NO)c1ccc(Cn2c(=O)[nH]c3cc(C(F)(F)F)ccc32)cc1]

According to another embodiment, the compound is

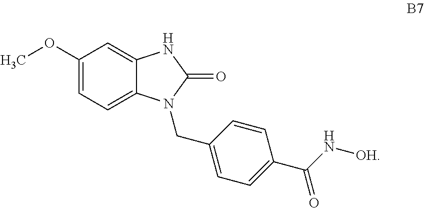 [SMILES: COc1ccc2c(c1)[nH]c(=O)n2Cc1ccc(C(=O)NO)cc1]

According to another embodiment, the compound is

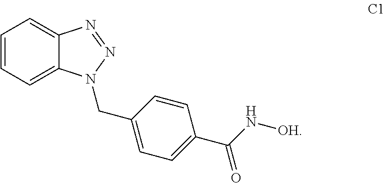 [SMILES: O=C(NO)c1ccc(Cn2nnc3ccccc32)cc1]

According to another embodiment, the compound is

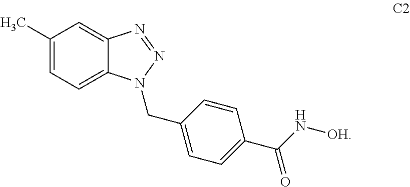 [SMILES: Cc1ccc2c(c1)nnn2Cc1ccc(C(=O)NO)cc1]

According to another embodiment, the compound is

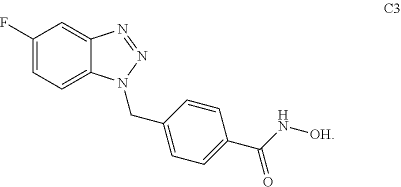 [SMILES: O=C(NO)c1ccc(Cn2nnc3cc(F)ccc32)cc1]

According to another embodiment, the compound is

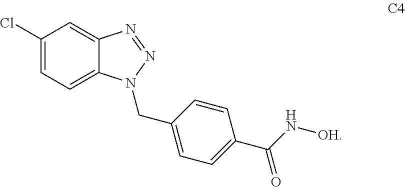 [SMILES: O=C(NO)c1ccc(Cn2nnc3cc(Cl)ccc32)cc1]

According to another embodiment, the compound is

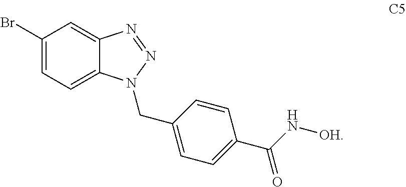 [SMILES: O=C(NO)c1ccc(Cn2nnc3cc(Br)ccc32)cc1]

According to another aspect, the present invention provides a composition for treating a histone deacetylase (HDAC)-associated disease, wherein the composition comprises (a) at least one compound of Formula I

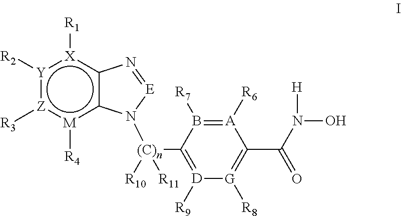

or a pharmaceutically acceptable salt thereof, wherein: each of X, Y, Z and M is independently C or N; each of R₁, R₂, R₃ and R₄ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₁, R₂, R₃ and R₄ is H or a substituent when X, Y, Z and M is carbon; E is C—R₅, or N; R₅ is H, OH, NH₂, amino optionally substituted by alkyl or aryl, CN, F, Cl, Br, I, C₁-C₆ perfluoroalkyl, O-alkyl, O-aryl, O-heteroaryl, NO₂, cycloalkyl, aryl, acyl, optionally substituted C₁-C₆ alkyl, C₂-C₆ alkene, or C₂-C₆ alkyne, wherein when R₅ is OH, the compound exists as a keto tautomer, as an enol tautomer or as a mixture of keto-enol tautomers; each of A, B, D, and G is independently C or N; each of R₆, R₇, R₈, and R₉ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₆, R₇, R₈ and R₉ is H or a substituent when A, B, D and G is carbon; each of R₁₀ and R₁₁ is independently H, alkyl, or aryl, wherein (C)ₙ optionally is a chiral center, wherein (C)ₙ can exist as both R and S enantiomers, with the proviso that when R₁₀ is H, R₁₁ is alkyl or aryl; and when R₁₁ is H, R₁₀ is alkyl or aryl; and n is 0, 1, 2, 3, 4, 5, 6, 7, 8, 9 or 10, wherein the compound is a histone deacetylatase (HDAC) inhibitor, and wherein the HDAC inhibitor inhibits histone deacetylating activity of at least one HDAC isoform selected from the group consisting of HDAC1, HDAC2, HDAC3, HDAC4, HDAC5, HDAC6, HDAC7, HDAC8, HDAC9, and a combination thereof; and (b) a pharmaceutically acceptable carrier. According to one embodiment, the HDAC inhibitor compound of formula I is a compound of Formula Ia:

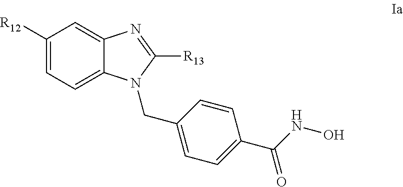

or a pharmaceutically acceptable salt thereof, wherein: R₁₂ is selected from the group consisting of H, alkyl, F, Cl, Br, I, and O-alkyl; and R₁₃ is selected from the group consisting of H and C₁-C₆ perfluoroalkyl. According to another embodiment, the HDAC inhibitor compound of Formula I is a compound of Formula Ib:

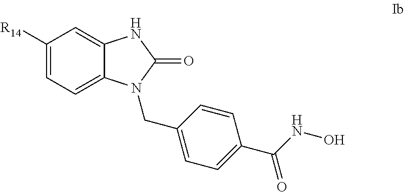

or a pharmaceutically acceptable salt thereof, wherein: R₁₄ is selected from the group consisting of H, alkyl, F, Cl, Br, I, O-alkyl, and C₁-C₆ perfluoroalkyl. According to another embodiment, the HDAC inhibitor compound of Formula I is a compound of Formula Ic:

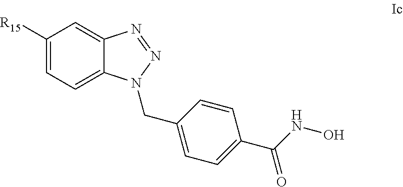

or a pharmaceutically acceptable salt thereof, wherein: R₁₅ is selected from the group consisting of H, alkyl, F, Cl, Br, I, and O-alkyl. According to another embodiment, the HDAC inhibitor compound inhibits the histone deacetylating activity of at least one HDAC isoform with an inhibition activity (IC₅₀) of from about 0.005 μM to about 2.76 μM. According to another embodiment, the HDAC inhibitor compound inhibits the histone deacetylating activity of HDAC6 with an inhibition activity (IC₅₀) from about 0.000001 μM to about 0.001 μM. According to another embodiment, the HDAC inhibitor compound is selective toward HDAC6. According to another embodiment, a ratio of the inhibitory activity (IC₅₀) of the HDAC inhibitor compound obtained in the presence of an HDAC isoform selected from the group consisting of HDAC1, HDAC2, HDAC3, HDAC4, HDAC5, HDAC7, HDAC8, and HDAC9, to the inhibition activity (IC₅₀) value of the HDAC inhibitor compound selective toward HDAC6 obtained in vitro in the presence of HDAC6 (in vitro selectivity value) has a value of at least 100. According to another embodiment, a ratio of the inhibitory activity (IC₅₀) of the HDAC inhibitor compound obtained in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor compound selective toward HDAC6 obtained in vitro in the presence of HDAC6 (in vitro selectivity value) has a value of at least 30,000. According to another embodiment, a ratio of the half-maximal dose response (EC50) value of acetylated histone obtained in cell with the HDAC inhibitor compound to the half-maximal dose response (EC50) value of acetylated tubulin obtained in cell with the HDAC inhibitor compound (in cell selectivity value) has a value of at least 2.0. According to another embodiment, a ratio of the half-maximal dose response (EC50) value of acetylated histone obtained in cell with the HDAC inhibitor compound to the half-maximal dose response (EC50) value of acetylated tubulin obtained in cell with the HDAC inhibitor compound (in cell selectivity value) has a value of at least 50.0. According to another embodiment, the HDAC inhibitor compound is selected from:

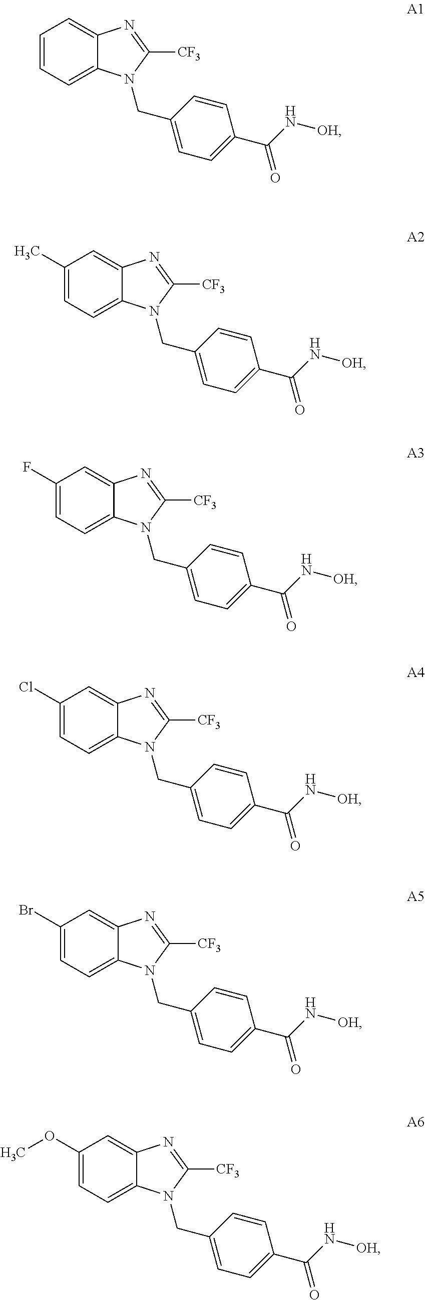 [SMILES: O=C(NO)c1ccc(Cn2c(C(F)(F)F)nc3ccccc32)cc1 ; Cc1ccc2c(c1)nc(C(F)(F)F)n2Cc1ccc(C(=O)NO)cc1 ; O=C(NO)c1ccc(Cn2c(C(F)(F)F)nc3cc(F)ccc32)cc1 ; O=C(NO)c1ccc(Cn2c(C(F)(F)F)nc3cc(Cl)ccc32)cc1 ; O=C(NO)c1ccc(Cn2c(C(F)(F)F)nc3cc(Br)ccc32)cc1 ; COc1ccc2c(c1)nc(C(F)(F)F)n2Cc1ccc(C(=O)NO)cc1]

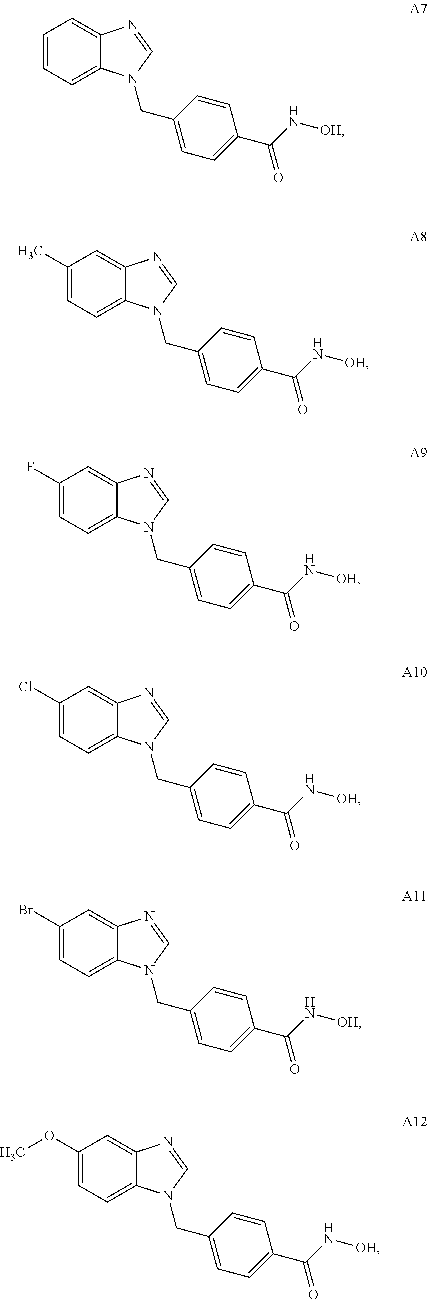 [SMILES: O=C(NO)c1ccc(Cn2cnc3ccccc32)cc1 ; Cc1ccc2c(c1)ncn2Cc1ccc(C(=O)NO)cc1 ; O=C(NO)c1ccc(Cn2cnc3cc(F)ccc32)cc1 ; O=C(NO)c1ccc(Cn2cnc3cc(Cl)ccc32)cc1 ; O=C(NO)c1ccc(Cn2cnc3cc(Br)ccc32)cc1 ; COc1ccc2c(c1)ncn2Cc1ccc(C(=O)NO)cc1]

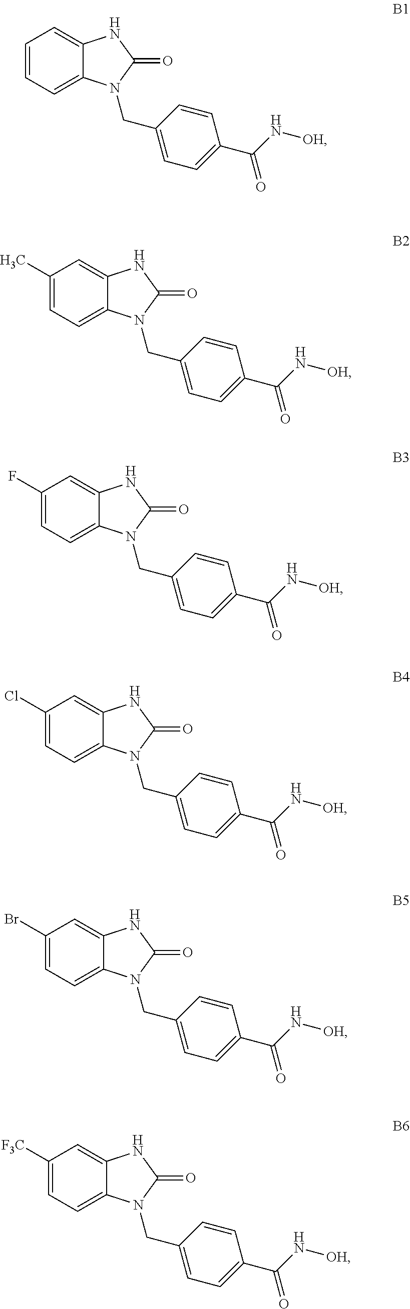 [SMILES: O=C(NO)c1ccc(Cn2c(=O)[nH]c3ccccc32)cc1 ; Cc1ccc2c(c1)[nH]c(=O)n2Cc1ccc(C(=O)NO)cc1 ; O=C(NO)c1ccc(Cn2c(=O)[nH]c3cc(F)ccc32)cc1 ; O=C(NO)c1ccc(Cn2c(=O)[nH]c3cc(Cl)ccc32)cc1 ; O=C(NO)c1ccc(Cn2c(=O)[nH]c3cc(Br)ccc32)cc1 ; O=C(NO)c1ccc(Cn2c(=O)[nH]c3cc(C(F)(F)F)ccc32)cc1]

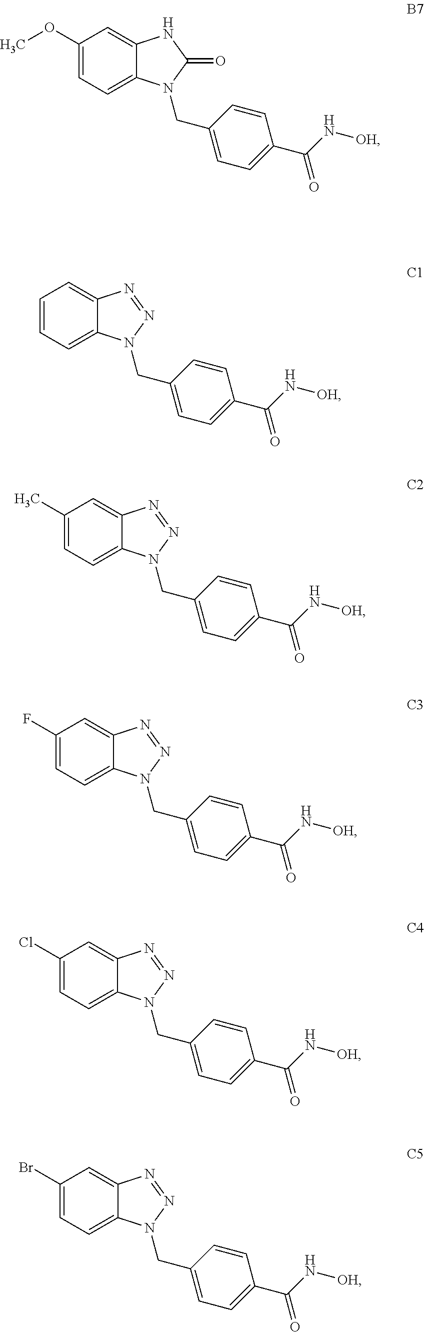 [SMILES: COc1ccc2c(c1)[nH]c(=O)n2Cc1ccc(C(=O)NO)cc1 ; O=C(NO)c1ccc(Cn2nnc3ccccc32)cc1 ; Cc1ccc2c(c1)nnn2Cc1ccc(C(=O)NO)cc1 ; O=C(NO)c1ccc(Cn2nnc3cc(F)ccc32)cc1 ; O=C(NO)c1ccc(Cn2nnc3cc(Cl)ccc32)cc1 ; O=C(NO)c1ccc(Cn2nnc3cc(Br)ccc32)cc1]

or a combination thereof. According to another embodiment, the HDAC inhibitor compound is

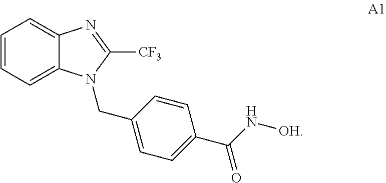 [SMILES: O=C(NO)c1ccc(Cn2c(C(F)(F)F)nc3ccccc32)cc1]

According to another embodiment, the HDAC inhibitor compound is

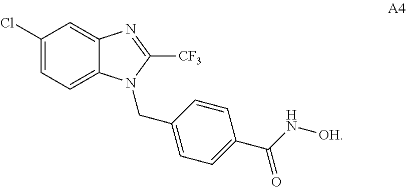 [SMILES: O=C(NO)c1ccc(Cn2c(C(F)(F)F)nc3cc(Cl)ccc32)cc1]

According to another embodiment, the HDAC inhibitor compound is

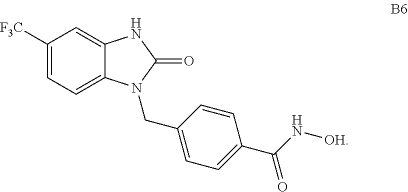 [SMILES: O=C(NO)c1ccc(Cn2c(=O)[nH]c3cc(C(F)(F)F)ccc32)cc1]

According to another aspect, the present invention provides a method of treating a histone deacetylase (HDAC)-associated disease, comprising: (a) providing at least one compound of Formula I:

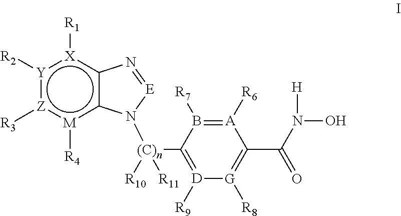

or a pharmaceutically acceptable salt thereof, wherein: each of X, Y, Z and M is independently C or N; each of R₁, R₂, R₃ and R₄ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₁, R₂, R₃ and R₄ is H or a substituent when X, Y, Z and M is carbon; E is C—R₅, or N; R₅ is H, OH, NH₂, amino optionally substituted by alkyl or aryl, CN, F, Cl, Br, I, C₁-C₆ perfluoroalkyl, O-alkyl, O-aryl, O-heteroaryl, NO₂, cycloalkyl, aryl, acyl, optionally substituted C₁-C₆ alkyl, C₂-C₆ alkene, or C₂-C₆ alkyne, wherein when R₅ is OH, the compound exists as a keto tautomer, as an enol tautomer or as a mixture of keto-enol tautomers; each of A, B, D, and G is independently C or N; each of R₆, R₇, R₈, and R₉ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₆, R₇, R₈ and R₉ is H or a substituent when A, B, D and G is carbon; each of R₁₀ and R₁₁ is independently H, alkyl, or aryl, wherein (C)ₙ optionally is a chiral center, wherein (C)ₙ can exist as both R and S enantiomers, with the proviso that when R₁₀ is H, R₁₁ is alkyl or aryl; and when R₁₁ is H, R₁₀ is alkyl or aryl; and n is 0, 1, 2, 3, 4, 5, 6, 7, 8, 9 or 10, wherein the compound is a histone deacetylatase (HDAC) inhibitor, and wherein the HDAC inhibitor inhibits histone deacetylating activity of at least one HDAC isoform selected from the group consisting of HDAC1, HDAC2, HDAC3, HDAC4, HDAC5, HDAC6, HDAC7, HDAC8, HDAC9, and a combination thereof; and (b) administering a composition to a subject with symptoms of the HDAC-associated disease, comprising a therapeutic amount of the HDAC inhibitor compound and a pharmaceutically acceptable carrier, wherein the therapeutic amount is effective to inhibit the activity of at least one HDAC isoform and in treating the symptoms of the HDAC-associated disease, wherein the therapeutic amount of the HDAC inhibitor compound is capable of achieving a half-maximal dose response (EC50) value of acetylated tubulin obtained in cell ranging between 0.05 μM to 0.5 μM, wherein the HDAC-associated disease is characterized by lower level of acetylated tubulin in cells isolated from the subject with symptoms of the HDAC-associated disease relative to the level of acetylated tubulin in cells isolated from a healthy subject, and wherein the HDAC-associated disease is selected from the group consisting of a cell proliferative disease, an autoimmune or inflammatory disorder, a neurodegenerative disease, or a combination thereof. According to one embodiment, the HDAC inhibitor compound of formula I is a compound of Formula Ia:

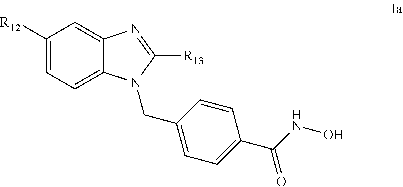

or a pharmaceutically acceptable salt thereof, wherein: R₁₂ is selected from the group consisting of H, alkyl, F, Cl, Br, I, and O-alkyl; and R₁₃ is selected from the group consisting of H and C₁-C₆ perfluoroalkyl. According to another embodiment, the HDAC inhibitor compound of Formula I is a compound of Formula Ib:

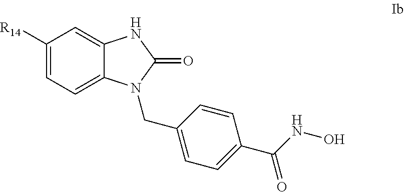

or a pharmaceutically acceptable salt thereof, wherein: R₁₄ is selected from the group consisting of H, alkyl, F, Cl, Br, I, O-alkyl, and C₁-C₆ perfluoroalkyl. According to another embodiment, the HDAC inhibitor compound of Formula I is a compound of Formula Ic:

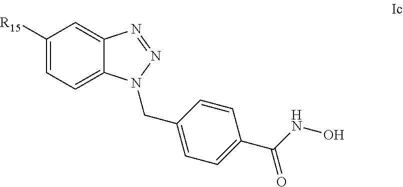

or a pharmaceutically acceptable salt thereof, wherein: R₁₅ is selected from the group consisting of H, alkyl, F, Cl, Br, I, and O-alkyl. According to another embodiment, the HDAC inhibitor compound inhibits the histone deacetylating activity of at least one HDAC isoform with an inhibition activity (IC₅₀) of from about 0.005 μM to about 2.76 μM. According to another embodiment, the HDAC inhibitor compound inhibits the histone deacetylating activity of HDAC6 with an inhibition activity (IC₅₀) from about 0.000001 μM to about 0.001 μM. According to another embodiment, the HDAC inhibitor compound is selective toward HDAC6. According to another embodiment, a ratio of the inhibitory activity (IC₅₀) of the HDAC inhibitor compound obtained in the presence of an HDAC isoform selected from the group consisting of HDAC1, HDAC2, HDAC3, HDAC4, HDAC5, HDAC7, HDAC8, and HDAC9, to the inhibition activity (IC₅₀) value of the HDAC inhibitor compound selective toward HDAC6 obtained in vitro in the presence of HDAC6 (in vitro selectivity value) has a value of at least 100. According to another embodiment, a ratio of the inhibitory activity (IC₅₀) the HDAC inhibitor compound obtained in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor compound selective toward HDAC6 obtained in vitro in the presence of HDAC6 (in vitro selectivity value) has a value of at least 30,000. According to another embodiment, a ratio of the half-maximal dose response (EC50) value of acetylated histone obtained in cell with the HDAC inhibitor compound to the half-maximal dose response (EC50) value of acetylated tubulin obtained in cell with the HDAC inhibitor compound (in cell selectivity value) has a value of at least 2.0. According to another embodiment, a ratio of the half-maximal dose response (EC50) value of acetylated histone obtained in cell with the HDAC inhibitor compound to the half-maximal dose response (EC50) value of acetylated tubulin obtained in cell with the HDAC inhibitor compound (in cell selectivity value) has a value of at least 50.0. According to another embodiment, the HDAC compound is selected from:

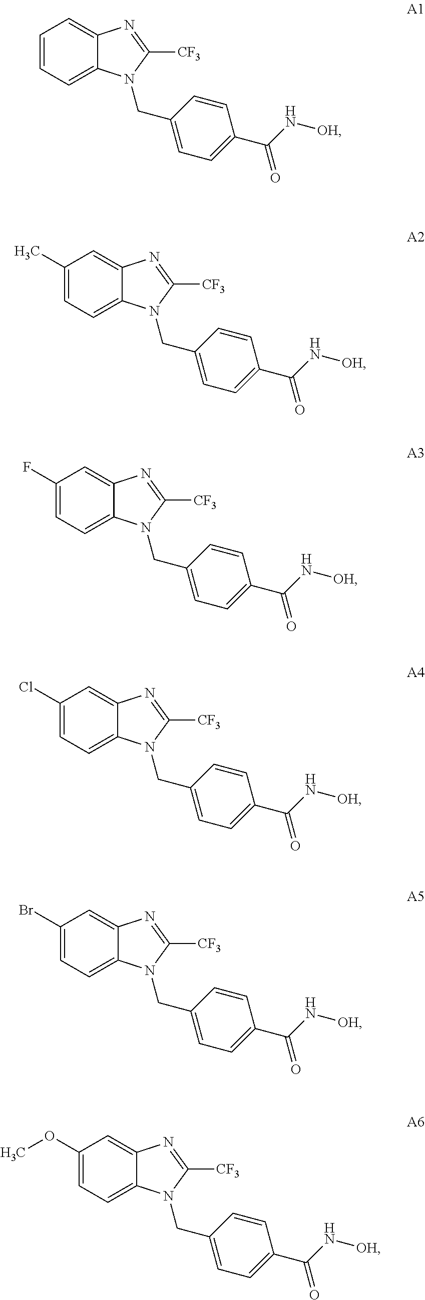 [SMILES: O=C(NO)c1ccc(Cn2c(C(F)(F)F)nc3ccccc32)cc1 ; Cc1ccc2c(c1)nc(C(F)(F)F)n2Cc1ccc(C(=O)NO)cc1 ; O=C(NO)c1ccc(Cn2c(C(F)(F)F)nc3cc(F)ccc32)cc1 ; O=C(NO)c1ccc(Cn2c(C(F)(F)F)nc3cc(Cl)ccc32)cc1 ; O=C(NO)c1ccc(Cn2c(C(F)(F)F)nc3cc(Br)ccc32)cc1 ; COc1ccc2c(c1)nc(C(F)(F)F)n2Cc1ccc(C(=O)NO)cc1]

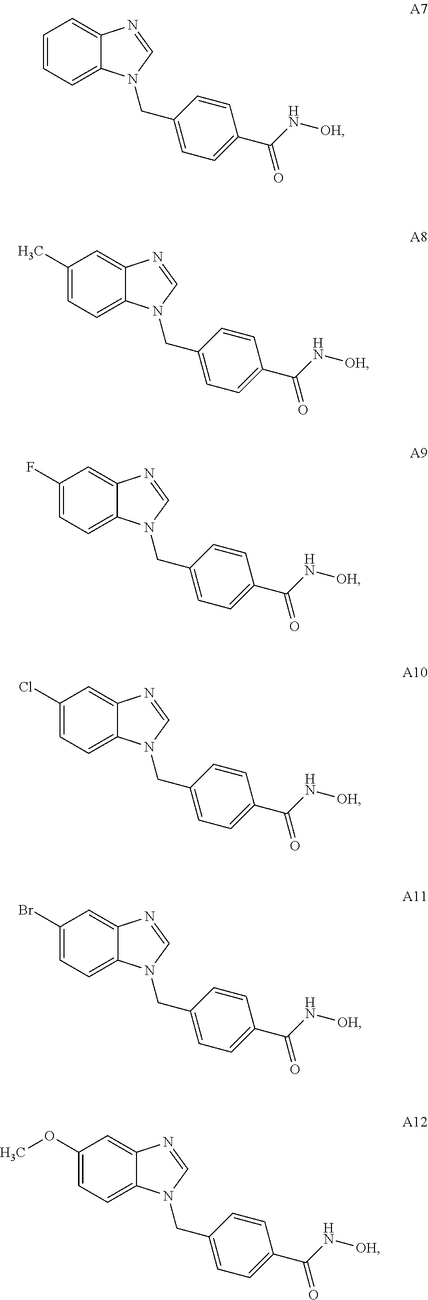 [SMILES: O=C(NO)c1ccc(Cn2cnc3ccccc32)cc1 ; Cc1ccc2c(c1)ncn2Cc1ccc(C(=O)NO)cc1 ; O=C(NO)c1ccc(Cn2cnc3cc(F)ccc32)cc1 ; O=C(NO)c1ccc(Cn2cnc3cc(Cl)ccc32)cc1 ; O=C(NO)c1ccc(Cn2cnc3cc(Br)ccc32)cc1 ; COc1ccc2c(c1)ncn2Cc1ccc(C(=O)NO)cc1]

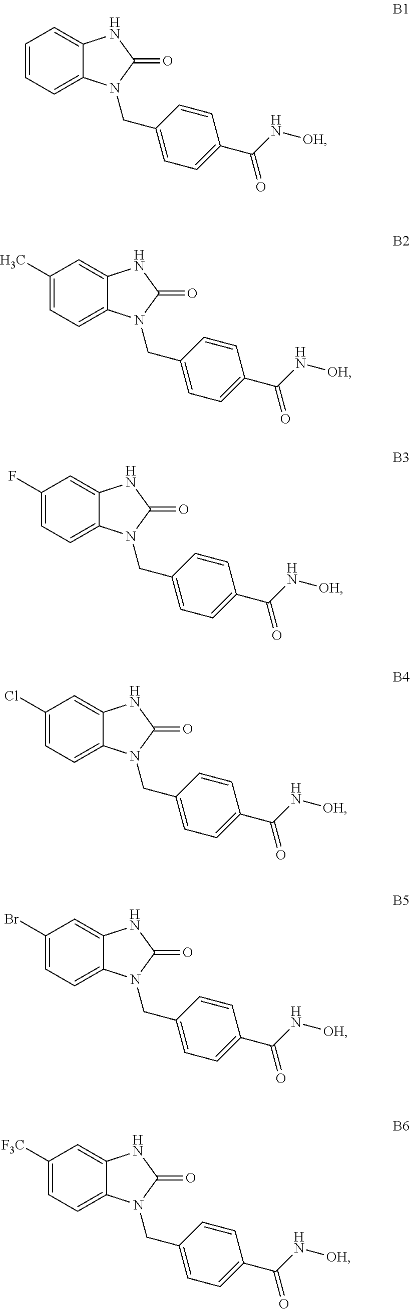 [SMILES: O=C(NO)c1ccc(Cn2c(=O)[nH]c3ccccc32)cc1 ; Cc1ccc2c(c1)[nH]c(=O)n2Cc1ccc(C(=O)NO)cc1 ; O=C(NO)c1ccc(Cn2c(=O)[nH]c3cc(F)ccc32)cc1 ; O=C(NO)c1ccc(Cn2c(=O)[nH]c3cc(Cl)ccc32)cc1 ; O=C(NO)c1ccc(Cn2c(=O)[nH]c3cc(Br)ccc32)cc1 ; O=C(NO)c1ccc(Cn2c(=O)[nH]c3cc(C(F)(F)F)ccc32)cc1]

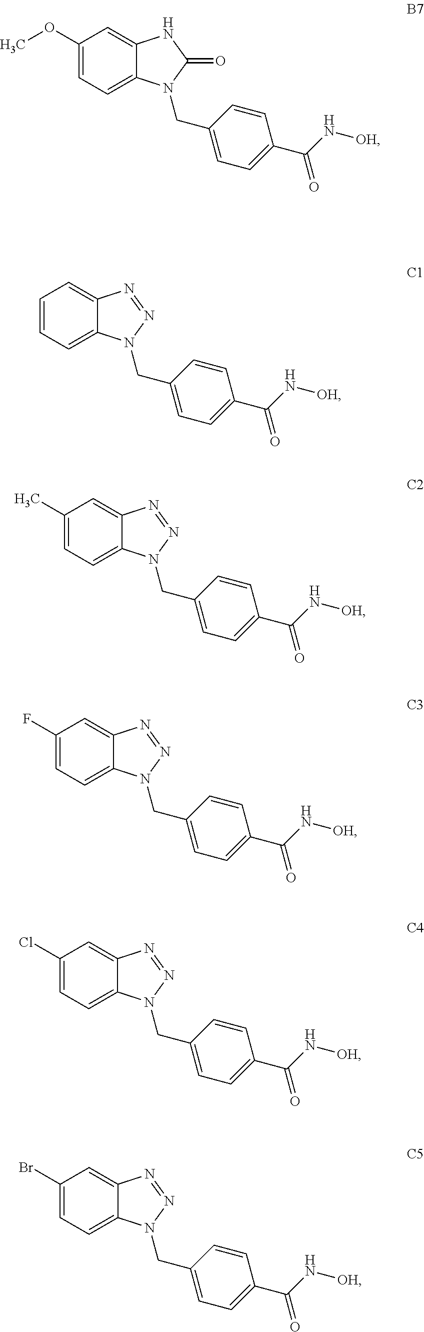 [SMILES: COc1ccc2c(c1)[nH]c(=O)n2Cc1ccc(C(=O)NO)cc1 ; O=C(NO)c1ccc(Cn2nnc3ccccc32)cc1 ; Cc1ccc2c(c1)nnn2Cc1ccc(C(=O)NO)cc1 ; O=C(NO)c1ccc(Cn2nnc3cc(F)ccc32)cc1 ; O=C(NO)c1ccc(Cn2nnc3cc(Cl)ccc32)cc1 ; O=C(NO)c1ccc(Cn2nnc3cc(Br)ccc32)cc1]

or a combination thereof. According to another embodiment, the HDAC inhibitor compound is

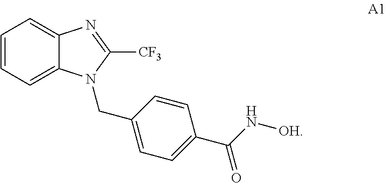 [SMILES: O=C(NO)c1ccc(Cn2c(C(F)(F)F)nc3ccccc32)cc1]

According to another embodiment, the HDAC inhibitor compound is

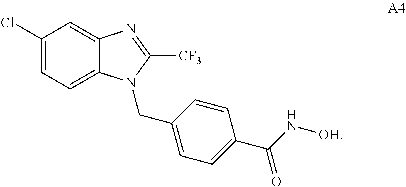 [SMILES: O=C(NO)c1ccc(Cn2c(C(F)(F)F)nc3cc(Cl)ccc32)cc1]

According to another embodiment, the HDAC inhibitor compound is

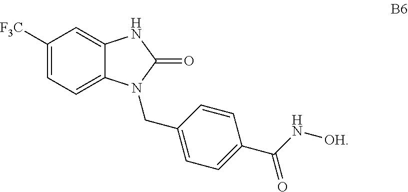 [SMILES: O=C(NO)c1ccc(Cn2c(=O)[nH]c3cc(C(F)(F)F)ccc32)cc1]

According to another embodiment, the cell proliferative disease is a cancer, selected from the group consisting of an ovarian cancer, a prostate cancer, a lung cancer, an acute myeloid leukemia, a multiple myeloma, a bladder carcinoma, a renal carcinoma, a breast carcinoma, a colorectal carcinoma, a neuroblastoma, a melanoma, a gastric cancer, or a combination thereof. According to another embodiment, the autoimmune or inflammatory disorder is selected from the group consisting of a rheumatoid arthritis, a psoriasis, an inflammatory bowel disease, a multiple sclerosis, a systemic lupus erthematosus, an airway hyperresponsiveness, a Crohn's disease, an ulcerative colitis, or a combination thereof. According to another embodiment, the neurodegenerative disorder is selected from the group consisting of a cerebral ischemia, a Huntington's disease, an amyotrophic lateral sclerosis, a spinal musclular atrophy, a Parkinson's disease, an Alzheimer's disease, or a combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows dose response curves obtained with HDAC inhibitors, A1, A2, A3, A4, A5, A6, A7, A8, A9, A10, A11, A12, B1, B2, B3, B4, B5, B6, B7, C1, C2, C3, C4, and C5 for inhibition of HDAC1.

FIG. 2 shows dose response curves obtained with HDAC inhibitors, A1, A2, A3, A4, A5, A6, A7, A8, A9, A10, A11, A12, B1, B2, B3, B4, B5, B6, B7, C1, C2, C3, C4, and C5 for inhibition of HDAC2.

FIG. 3 shows dose response curves obtained with HDAC inhibitors, A1, A2, A3, A4, A5, A6, A7, A8, A9, A10, A11, A12, B1, B2, B3, B4, B5, B6, B7, C1, C2, C3, C4, and C5 for inhibition of HDAC3.

FIG. 4 shows dose response curves obtained with HDAC inhibitors, A1, A2, A3, A4, A5, A6, A7, A8, A9, A10, A11, A12, B1, B2, B3, B4, B5, B6, B7, C1, C2, C3, C4, and C5 for inhibition of HDAC4.

FIG. 5 shows dose response curves obtained with HDAC inhibitors, A1, A2, A3, A4, A5, A6, A7, A8, A9, A10, A11, A12, B1, B2, B3, B4, B5, B6, B7, C1, C2, C3, C4, and C5 for inhibition of HDAC5.

FIG. 6 shows dose response curves obtained with HDAC inhibitors, A1, A2, A3, A4, A5, A6, A7, A8, A9, A10, A11, A12, B1, B2, B3, B4, B5, B6, B7, C1, C2, C3, C4, and C5 for inhibition of HDAC6.

FIG. 7 shows dose response curves obtained with HDAC inhibitors, A1, A2, A3, A4, A5, A6, A7, A8, A9, A10, A11, A12, B1, B2, B3, B4, B5, B6, B7, C1, C2, C3, C4, and C5 for inhibition of HDAC7.

FIG. 8 shows dose response curves obtained with HDAC inhibitors, A1, A2, A3, A4, A5, A6, A7, A8, A9, A10, A11, A12, B1, B2, B3, B4, B5, B6, B7, C1, C2, C3, C4, and C5 for inhibition of HDAC8.

FIG. 9 shows dose response curves obtained with HDAC inhibitors, A1, A2, A3, A4, A5, A6, A7, A8, A9, A10, A11, A12, B1, B2, B3, B4, B5, B6, B7, C1, C2, C3, C4, and C5 for inhibition of HDAC9.

FIG. 10 shows dose response curves obtained with HDAC inhibitors, A1, A2, A3, A4, A5, A6, A7, A8, A9, A10, A11, A12, B1, B2, B3, B4, B5, B6, B7, C1, C2, C3, C4, and C5 for inhibition of HDAC1, HDAC2, HDAC3 and HDAC6.

FIG. 11 shows plots of EC50 (μM) values obtained for half-maximal induction of acetylated histones (Squares) or acetylated tubulin (Circles) as measured by quantitative, automated epifluorescence microscopy, with a control compound, SAHA in (A), HDAC inhibitor A4 in (B), HDAC inhibitor A1 in (C), and HDAC inhibitor B6 in (D).

### GLOSSARY

The term “absolute configuration” refers to the spatial arrangement of the atoms of a chiral molecular entity (or group) and its stereochemical description, for example, R or S.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “active” as used herein refers to having pharmacological or biological activity or affect.

The terms “active agent” or “active ingredient” as used herein refer to the ingredient, component or constituent of the compositions of the present invention responsible for the intended therapeutic effect.

The term “active ingredient” (“AI”, “active pharmaceutical ingredient”, or “bulk active”) is the substance in a drug that is pharmaceutically active. As used herein, the phrase “additional active ingredient” refers to an agent, other than a compound of the inventive composition that exerts a pharmacological, or any other beneficial activity.

The term “acyl” as used herein refers to the group RaC(O)—, where Ra is alkyl, alkenyl, alkynyl, cycloalkyl, cycloalkenyl, or heterocyclyl.

The term “acyloxy” as used herein refers to the group RaC(O)O—, where Ra is alkyl, alkenyl, alkynyl, cycloalkyl, cycloalkenyl, or heterocyclyl

The term “administering” as used herein includes in vivo administration, as well as administration directly to tissue ex vivo. Generally, compositions may be administered systemically either orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be locally administered by means such as, but not limited to, injection, implantation, grafting, topical application, or parenterally.

The term “alkenyl,” or “alkene” as used herein, denotes a monovalent, straight (unbranched) or branched hydrocarbon chain having 2 to 10 carbon atoms and one or more double bonds therein where the double bond can be unconjugated or conjugated to another unsaturated group (e.g., a polyunsaturated alkenyl) and can be unsubstituted or substituted, with multiple degrees of substitution being allowed. It may be optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkenyl” group may contain one or more O, S, S(O), or S(O)₂ atoms. For example, and without limitation, the alkenyl can be vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, decenyl, undecenyl, dodecenyl, heptadecenyl, octadecenyl, nonadecenyl, eicosenyl, heneicosenyl, docosenyl, tricosenyl, tetracisenyl, pentacosenyl, phytyl, the branched chain isomers thereof, and polyunsaturated alkenes including octadec-9,12,-dienyl, octadec-9,12,15-trienyl, and eicos-5,8,11,14-tetraenyl.

As used herein, the term “alkenylene” refers to a straight or branched chain divalent hydrocarbon radical having from 2 to 10 carbon atoms and one or more carbon-carbon double bonds, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkenylene” group may contain one or more O, S, S(O), or S(O)₂ atoms. Examples of “alkenylene” as used herein include, but are not limited to, ethene-1,2-diyl, propene-1,3-diyl, methylene-1,1-diyl, and the like.

The term “alkenyloxy” as used herein refers to the group RaO—, where Ra is alkenyl.

The term “alkenylsulfanyl” as used herein refers to the group RaS—, where Ra is alkenyl.

The term “alkenylsulfenyl” as used herein refers to the group RaS(O)—, where Ra is alkenyl.

The term “alkenylsulfonyl” as used herein refers to the group RaSO₂—, where Ra is alkenyl.

The term “alkoxy” as used herein refers to the group RaO—, where Ra is alkyl.

The term “alkoxycarbonyl” as used herein refers to the group RaOC(O)—, where Ra is alkyl.

As used herein, the term “alkyl” refers to a straight or branched chain hydrocarbon having from 1 to 10 carbon atoms carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkyl” group may contain one or more O, S, S(O), or S(O)₂ atoms. Examples of “alkyl” as used herein include, but are not limited to, methyl, ethyl, propyl, decyl, undecyl, octadecyl, nonadecyl, eicosyl, heneicosyl, decosyl, tricosyl, tetracosyl, and pentacosyl, n-butyl, t-butyl, n-pentyl, isobutyl, and isopropyl, and the like.

The term “alkylene” as used herein refers to a straight or branched chain divalent hydrocarbon radical having from 1 to 10 carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkylene” group may contain one or more O, S, S(O), or S(O)₂ atoms. Examples of “alkylene” as used herein include, but are not limited to, methylene, ethylene, and the like.

The term “alkylsulfanyl” as used herein refers to the group RaS—, where Ra is alkyl.

The term “alkylsulfenyl” as used herein refers to the group RaS(O)—, where Ra is alkyl.

The term “alkylsulfonyl” as used herein refers to the group RaSO₂—, where Ra is alkyl.

As used herein, the term “alkynyl” or “alkyne” refers to a hydrocarbon radical having from 2 to 10 carbon atoms and at least one carbon-carbon triple bond, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkynyl” group may contain one or more O, S, S(O), or S(O)₂ atoms.

As used herein, the term “alkynylene” refers to a straight or branched chain divalent hydrocarbon radical having from 2 to 10 carbon atoms and one or more carbon-carbon triple bonds, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Such an “alkynylene” group may contain one or more O, S, S(O), or S(O)₂ atoms. Examples of “alkynylene” as used herein include, but are not limited to, ethyne-1,2-diyl, propyne-1,3-diyl, and the like.

The term “alkynyloxy” as used herein refers to the group RaO—, where Ra is alkynyl.

The term “alkynylsulfanyl” as used herein refers to the group RaS—, where Ra is alkynyl.

The term “alkynylsulfenyl” as used herein refers to the group RaS(O)—, where Ra is alkynyl.

The term “alkynylsulfonyl” as used herein refers to the group RaSO₂—, where Ra is alkynyl.

The term “amino” as used herein refers to the substituent —NH₂.

The term “aminosulfonyl” as used herein refers to the substituent —SO₂NH₂.

The term “anesthetic agents” refers to agents that resulting in a reduction or loss of sensation.

The term “antibiotic agent” as used herein means any of a group of chemical substances having the capacity to inhibit the growth of, or to destroy bacteria, and other microorganisms, used chiefly in the treatment of infectious diseases.

The term “anti-fungal agent” as used herein means any of a group of chemical substances having the capacity to inhibit the growth of or to destroy fungi.

The term “antihistamine agent” as used herein refers to any of various compounds that counteract histamine in the body and that are used for treating allergic reactions (such as hay fever) and cold symptoms.

The term “anti-inflammatory agent” as used herein refers to an agent that reduces inflammation. The term “steroidal anti-inflammatory agent”, as used herein, refer to any one of numerous compounds containing a 17-carbon 4-ring system and includes the sterols, various hormones (as anabolic steroids), and glycosides. The term “non-steroidal anti-inflammatory agents” refers to a large group of agents that are aspirin-like in their action, including ibuprofen (Advil)®, naproxen sodium (Aleve)®, and acetaminophen (Tylenol)®.

The term “an anti-oxidant agent” as used herein refers to a substance that inhibits oxidation or reactions promoted by oxygen or peroxides.

The term “anti-protozoal agent” as used herein means any of a group of chemical substances having the capacity to inhibit the growth of or to destroy protozoans used chiefly in the treatment of protozoal diseases.

The term “antipruritic agents” as used herein refers to those substances that reduce, eliminate or prevent itching.

The term “anti-viral agent” as used herein means any of a group of chemical substances having the capacity to inhibit the replication of or to destroy viruses used chiefly in the treatment of viral diseases.

The term “aroyl” as used herein refers to the group RaC(O)—, where Ra is aryl.

The term “aroyloxy” as used herein refers to the group RaC(O)O—, where Ra is aryl.

The term “aryl” as used herein refers to a benzene ring or to an optionally substituted benzene ring system fused to one or more optionally substituted benzene rings, with multiple degrees of substitution being allowed. Substituents include, but are not limited to, lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of aryl include, but are not limited to, phenyl, 2-napthyl, 1-naphthyl, 1-anthracenyl, and the like.

It should be understood that wherever the terms “alkyl” or “aryl” or either of their prefix roots appear in a name of a substituent, they are to be interpreted as including those limitations given above for alkyl and aryl. Designated numbers of carbon atoms (e.g. C₁₋₆) shall refer independently to the number of carbon atoms in an alkyl, alkenyl or alkynyl or cyclic alkyl moiety or to the alkyl portion of a larger substituent in which the term “alkyl” appears as its prefix root.

As used herein, the term “arylene” refers to a benzene ring diradical or to a benzene ring system diradical fused to one or more optionally substituted benzene rings, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, silyloxy optionally substituted by alkoxy, alkyl, or aryl, silyl optionally substituted by alkoxy, alkyl, or aryl, nitro, cyano, halogen, or lower perfluoroalkyl, multiple degrees of substitution being allowed. Examples of “arylene” include, but are not limited to, benzene-1,4-diyl, naphthalene-1,8-diyl, and the like.

The term “asymmetric” as used herein refers to lacking all symmetry elements (other than the trivial one of a one-fold axis of symmetry), i.e., belonging to the symmetry of point group C1. The term has been used loosely (and incorrectly) to describe the absence of a rotation-reflection axis (alternating axis) in a molecule, i.e., as meaning chiral, and this usage persists in the traditional terms such as, but not limited to, asymmetric carbon atom, asymmetric synthesis, and asymmetric induction.

The term “benign tumor” as used herein refers to a tumor that is differentiated, localized and non-metastatic and does not contain uncontrollably dividing cells.

The term “bioavailability” refers to the rate and extent to which the active drug ingredient or therapeutic moiety is absorbed into the systemic circulation from an administered dosage form as compared to a standard or control.

The term “binder” refers to substances that bind or “glue” powders together and make them cohesive by forming granules, thus serving as the “adhesive” in the formulation. Binders add cohesive strength already available in the diluent or bulking agent. Suitable binders include sugars such as sucrose; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2% to about 20% by weight of the composition, more preferably from about 3% to about 10% by weight, even more preferably from about 3% to about 6% by weight.

The term “capsule” refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

The term “carbamoyl” as used herein refers to the substituent —C(O)NH₂.

The term “carboxy” as used herein refers to the substituent —COOH.

The terms “cis” and “trans” are descriptors which show the relationship between two ligands attached to separate atoms that are connected by a double bond or are contained in a ring. The two ligands are said to be located cis to each other if they lie on the same side of a plane. If they are on opposite sides, their relative position is described as trans. The appropriate reference plane of a double bond is perpendicular to that of the relevant σ-bonds and passes through the double bond. For a ring (the ring being in a conformation, real or assumed, without re-entrant angles at the two substituted atoms) it is the mean place of the ring(s). For alkenes the terms cis and trans may be ambiguous and have therefore generally have been replaced by the E, Z convention for the nomenclature of organic compounds. If there are more than two entities attached to the ring the use of cis and trans requires the definition of a reference substituent (see IUPAC, Nomenclature of Organic Chemistry, Sections A, B, C, D, E, F and H, Pergamon Press, 1979, p. 478, Rule E-2.3.3, E-2.3.4; IUPAC, A Guide to IUPAC Nomenclature of Organic Chemistry, Blackwell Scientific Publications, 1993, pp. 149-151, Rule R-7.1.1).

The term “carrier” as used herein refers to an organic or inorganic ingredient, natural or synthetic, with which the active ingredient is combined to facilitate the application.

The terms “cis-trans isomers” refer to stereoisomeric olefins or cycloalkanes (or hetero-analogues) which differ in the positions of atoms (or groups) relative to a reference plane: in the cis-isomer the atoms are on the same side, in the trans-isomer they are on opposite sides. According to this definition, cis-trans isomerism is a form of diastereoisomerism. For example:

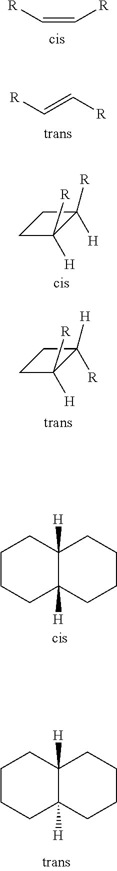

The term “chemotherapetic agent” refers to chemicals useful in the treatment or control of a disease.

The term “chiral” is used to describe asymmetric molecules (with four different substituent groups) that are nonsuperposable since they are mirror images of each other and therefore has the property of chirality. Such molecules are also called enantiomers and are characterized by optical activity.

The term “chirality” refers to the geometric property of a rigid object (or spatial arrangement of points or atoms) of being non-superposable on its mirror image; such an object has no symmetry elements of the second kind (a mirror plane, 6=51, a center of inversion, i=S2, a rotation-reflection axis, S2n). If the object is superposable on its mirror image the object is described as being achiral.

The term “chirality axis” refers to an axis about which a set of ligands is held so that it results in a spatial arrangement which is not superposable on its mirror image. For example, with an allene abC═C═Ccd the chiral axis is defined by the C═C═C bonds; and with an ortho-substituted biphenyl C-1, C-1′, C-4 and C-4′ lie on the chiral axis.

The term “chirality center” or a “chiral center” refers to an atom holding a set of ligands in a spatial arrangement, which is not superposable on its mirror image. A chirality center may be considered a generalized extension of the concept of the asymmetric carbon atom to central atoms of any element.

The terms “chiroptic” or “chiroptical” refer to the optical techniques (using refraction, absorption or emission of anisotropic radiation) for investigating chiral substances (for example, measurements of optical rotation at a fixed wavelength, optical rotary dispersion (ORD), circular dichroism (CD) and circular polarization of luminescence (CPL).

The term “chirotopic” refers to the an atom (or point, group, face, etc. in a molecular model) that resides within a chiral environment. One that resides within an achiral environment has been called achirotopic.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

The term “coloring agents” refers to excipients that provide coloration to the composition or the dosage form. Such excipients can include food grade dyes and food grade dyes adsorbed onto a suitable adsorbent such as clay or aluminum oxide. The amount of the coloring agent can vary from about 0.1% to about 5% by weight of the composition, preferably from about 0.1% to about 1%.

The term “condition”, as used herein, refers to a variety of health states and is meant to include disorders or diseases caused by any underlying mechanism or disorder, injury, and the promotion of healthy tissues and organs. Exemplary conditions include, but are not limited to, a variety of conditions related to HDACs. This term is meant to include disorders or diseases, associated with HDAC1, HDAC2, HDAC3, HDAC4, HDAC5, HDAC6, HDAC7, HDAC8, HDAC9, HDAC10 or HDAC11.

The term “configuration” refers to the three-dimensional shape of a molecule. In order to represent three-dimensional configurations on a two-dimensional surface, perspective drawings in which the direction of a bond is specified by the line connecting the bonded atoms are used.

The terms “contain” or “containing” can as used herein refers to in-line substitutions at any position along the above defined alkyl, alkenyl, alkynyl or cycloalkyl substituents with one or more of any of O, S, SO, SO₂, N, or N-alkyl, including, for example, —CH₂—O—CH₂—, CH₂ SO₂ CH₂—, CH₂ NH CH₃ and so forth.

The term “controlled release” is intended to refer to any drug-containing formulation in which the manner and profile of drug release from the formulation are controlled. This refers to immediate as well as non-immediate release formulations, with non-immediate release formulations including, but not limited to, sustained release and delayed release formulations.

The term “cyano” as used herein refers to the substituent —CN.

As used herein, “cycloalkyl” (used interchangeably with “aliphatic cyclic” herein) refers to a alicyclic hydrocarbon group optionally possessing one or more degrees of unsaturation, having from three to twelve carbon atoms, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. “Cycloalkyl” includes by way of example cyclopropyl, cyclobutyl, cyclopentyl, cyclohexyl, cycloheptyl, or cyclooctyl, and the like.

As used herein, the term “cycloalkylene” refers to an non-aromatic alicyclic divalent hydrocarbon radical having from three to twelve carbon atoms and optionally possessing one or more degrees of unsaturation, optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Examples of “cycloalkylene” as used herein include, but are not limited to, cyclopropyl-1,1-diyl, cyclopropyl-1,2-diyl, cyclobutyl-1,2-diyl, cyclopentyl-1,3-diyl, cyclohexyl-1,4-diyl, cycloheptyl-1,4-diyl, or cyclooctyl-1,5-diyl, and the like.

The term “delayed release” is used herein in its conventional sense to refer to a drug formulation in which there is a time delay between administration of the formulation and the release of the drug there from. “Delayed release” may or may not involve gradual release of drug over an extended period of time, and thus may or may not be “sustained release.”

The term “derivative” as used herein refers to a compound obtained from, or regarded as derived from, or produced by modification of, another and containing essential elements of the parent substance. The term “variant” as used herein refers to a compound or substance that deviates or differs from a standard. Generally, variants are slightly different from standards.

The term “diastereoisomerism” refers to stereoisomerism other than enantiomerism. Diastereoisomers (or diastereomers) are stereoisomers not related as mirror images. Diastereoisomers are characterized by differences in physical properties, and by some differences in chemical behavior towards achiral as well as chiral reagents. Diastereomers have similar chemical properties, since they are members of the same family. Their chemical properties are not identical, however. Diastereomers have different physical properties: different melting points, boiling points solubilities in a given solvent, densities, refractive indexes, and so on. Diastereomers also differ in specific rotation; they may have the same or opposite signs of rotation, or some may be inactive. The presence of two chiral centers can lead to the existence of as many as four stereoisomers. For compounds containing three chiral centers, there could be as many as eight stereoisomers; for compounds containing four chiral centers, there could be as many as sixteen stereoisomers, and so on. The maximum number of stereoisomers that can exist is equal to 2n, where n is the number of chiral centers. The term “diastereotopic” refers to constitutionally equivalent atoms or groups of a molecule which are not symmetry related. Replacement of one of two diastereotopic atoms or groups results in the formation of one of a pair of diastereoisomers. For example, the two hydrogen atoms of the methylene group

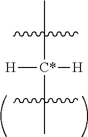

are diastereotopic.

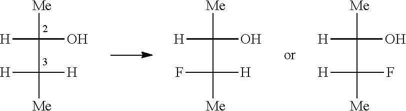 [SMILES: CCC(C)O ; CC(O)C(C)F ; CC(O)C(C)F]

The term “diluent” refers to substances that usually make up the major portion of the composition or dosage form. Exemplary diluents include, but are not limited to, sugars such as lactose, sucrose, mannitol and sorbitol; starches derived from wheat, corn, rice and potato; and celluloses such as microcrystalline cellulose. The amount of diluent in the composition can range from about 10% to about 90% by weight of the total composition, preferably from about 25% to about 75%, more preferably from about 30% to about 60% by weight, even more preferably from about 12% to about 60%.

As used herein, the term “direct bond”, where part of a structural variable specification, refers to the direct joining of the substituents flanking (preceding and succeeding) the variable taken as a “direct bond”.

The term “disintegrant” refers to materials added to the composition to help it break apart (disintegrate) and release the medicaments. Suitable disintegrants include starches; “cold water soluble” modified starches such as sodium carboxymethyl starch; natural and synthetic gums such as locust bean, karaya, guar, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2 to about 15% by weight of the composition, more preferably from about 4 to about 10% by weight.

The term “disease” or “disorder”, as used herein, refers to an impairment of health or a condition of abnormal functioning.

The term “drug” as used herein refers to a therapeutic agent or any substance used in the prevention, diagnosis, alleviation, treatment, or cure of disease.

The term “EC50” as used herein refers to the molar concentration of an agonist that produces 50% of the maximum possible response for that agonist.

The term “enantiomer” as used herein refers to one of a pair of optical isomers containing one or more asymmetric carbons (C*) whose molecular configurations have left- and right-hand (chiral) configurations. Enantiomers have identical physical properties, except as to the direction of rotation of the plane of polarized light. For example, glyceraldehyde and its mirror image have identical melting points, boiling points, densities, refractive indexes, and any other physical constant one might measure, expect that they are non-superimposable mirror images and one rotates the plane-polarized light to the right, while the other to the left by the same amount of rotation.

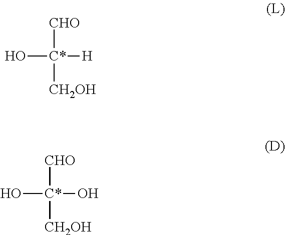

As used herein, the term “enzymatic activity” refers to the amount of substrate consumed (or product formed) in a given time under given conditions. Enzymatic activity also may be referred to as “turnover number.”

The term “ethoxy” as used herein refers to the substituent —O—CH₂CH₃.

The term “glidant” refers to material that prevents caking and improve the flow characteristics of granulations, so that flow is smooth and uniform. Suitable glidants include silicon dioxide and talc. The amount of glidant in the composition can range from about 0.1% to about 5% by weight of the total composition, preferably from about 0.5% to about 2% by weight.

The term “halogen” or “halo” as used herein includes iodine, bromine, chlorine and fluorine.

The term “heteroaroyl” as used herein refers to the group RaC(O)—, where Ra is heteroaryl.

The term “heteroaroyloxy” as used herein refers to the group RaC(O)O—, where Ra is heteroaryl.

The term “hormone” as used herein refers to natural substances produced by organs of the body that travel by blood to trigger activity in other locations or their synthetic analogs.

The term “IC₅₀ value” as used herein refers to the concentration of the HDAC inhibitor that results in 50% inhibition of HDAC activity.

The term “in cell selectivity value” as used herein refers to the ratio of the half-maximal dose response (EC50) value of acetylated histone obtained in cell with a HDAC inhibitor to the half-maximal dose response (EC50) value of acetylated tubulin obtained in cell with the HDAC inhibitor.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “inhibiting” as used herein refers to reducing or modulating the chemical or biological activity of a substance or compound.

The term “injury,” as used herein, refers to damage or harm to a structure or function of the body caused by an outside agent or force, which may be physical or chemical.

The term “in vitro selectivity value” as used herein refers to the ratio of the inhibition activity (IC₅₀) value of a HDAC inhibitor obtained in vitro in the presence of a HDAC isoform to the inhibition activity (IC₅₀) value of the HDAC inhibitor obtained in the presence of HDAC6.

The term “isomer” as used herein refers to one of two or more molecules having the same number and kind of atoms and hence the same molecular weight, but differing in respect to the arrangement or configuration of the atoms. Stereoisomers are isomers that are different from each other only in the way the atoms are oriented in space (but are like one another with respect to which atoms are joined to which other atoms).

The term “long-term” release, as used herein, means that the implant is constructed and arranged to deliver therapeutic levels of the active ingredient for at least 7 days, and preferably about 30 days to about 60 days.

The term “lubricant” refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d′1-leucine. Lubricants are usually added at the very last step before compression, since they must be present on the surfaces of the granules and in between them and the parts of the tablet press. The amount of lubricant in the composition can range from about 0.2% to about 5% by weight of the composition, preferably from about 0.5% to about 2%, more preferably from about 0.3% to about 1.5% by weight.

The term “malignant tumor” as used herein refers to tumor that is differentiated, and contain uncontrollably dividing cells. Such a tumor may be primary or secondary. A primary tumor refers to a malignant tumor that is localized at a site from where it arises, while a secondary tumor refers to malignant tumors that has metastasized from its cite of origin.

The term “mercapto” as used herein refers to the substituent —SH.

The term “methoxy” as used herein refers to the substituent —O—CH₃.

The term “modify” as used herein means to change, vary, adjust, temper, alter, affect or regulate to a certain measure or proportion in one or more particulars.

The term “modifying agent” as used herein refers to a substance, composition, extract, botanical ingredient, botanical extract, botanical constituent, therapeutic component, active constituent, therapeutic agent, drug, metabolite, active agent, protein, non-therapeutic component, non-active constituent, non-therapeutic agent, or non-active agent that reduces, lessens in degree or extent, or moderates the form, symptoms, signs, qualities, character or properties of a condition, state, disorder, disease, symptom or syndrome.

The term “modulate” as used herein means to regulate, alter, adapt, or adjust to a certain measure or proportion.

The terms “moiety” or “part” as used herein refer to functional groups of a molecule.

The term “O-linked moiety” means a moiety that is bonded through an oxygen atom. Thus, when an R group is an O-linked moiety, that R is bonded through oxygen and it thus can be an ether, an ester (e.g., —O—C(O)-optionally substituted alkyl), a carbonate or a carbamate (e.g., —O—C(O)—NH₂ or —O—C(O)—NH-optionally substituted alkyl). Similarly, the term “S-linked moiety” means a moiety that is bonded through a sulfur atom. Thus, when an R group is an S-linked moiety, that R is bonded through sulfur and it thus can be a thioether (e.g., —S-optionally substituted alkyl), a thioester (—S—C(O)-optionally substituted alkyl) or a disulfide (e.g., —S—S-optionally substituted alkyl). The term “N-linked moiety” means a moiety that is bonded through a nitrogen atom. Thus, when an R group is an N-linked moiety, the R group is bonded through nitrogen and one or more of these can thus be an N-linked amino acid such as —NH—CH₂—COOH, a carbamate such as —NH—C(O)—O-optionally substituted alkyl, an amine such as —NH-optionally substituted alkyl, an amide such as —NH—C(O)-optionally substituted alkyl or —N₃. The term “C-linked moiety” means a moiety that is bonded through a carbon atom. When one or more R group is bonded through carbon, one or more of these thus can be -optionally substituted alkyl such as —CH₂—CH₂—O—CH₃, —C(O)-optionally substituted alkyl hydroxyalkyl, mercaptoalkyl, aminoalkyl or ═CH-optionally substituted alkyl.

The term “optical rotation” refers to the change of direction of the plane of polarized light to either the right or the left as it passes through a molecule containing one or more asymmetric carbon atoms or chirality centers. The direction of rotation, if to the right, is indicated by either a plus sign (+) or a d−; if to the left, by a minus (−) or an l−. Molecules having a right-handed configuration (D) usually are dextrorotatory, D(+), but may be levorotatory, L(−). Molecules having left-handed configuration (L) are usually levorotatory, L(−), but may be dextrorotatory, D(+). Compounds with this property are said to be optically active and are termed optical isomers. The amount of rotation of the plane of polarized light varies with the molecule but is the same for any two isomers, though in opposite directions.

As used herein, the term “optionally” means that the subsequently described event(s) may or may not occur, and includes both event(s) which occur and events that do not occur.

The term “oral gel” refers to the active ingredients dispersed or solubilized in a hydrophillic semi-solid matrix.

The term “oxo” as used herein refers to the substituent═O.

The term “parenteral” as used herein refers to introduction into the body by way of an injection (i.e., administration by injection), including, for example, subcutaneously (i.e., an injection beneath the skin), intramuscularly (i.e., an injection into a muscle); intravenously (i.e., an injection into a vein), intrathecally (i.e., an injection into the space around the spinal cord or under the arachnoid membrane of the brain), intrasternal injection, or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersing or wetting agents and suspending agents.

The term “particles” as used herein refers to nano or microparticles (or in some instances larger) that may contain in whole or in part the HDAC inhibitor or the other therapeutic agent(s) as described herein.

The term “pharmaceutical composition” as used herein refers to a preparation comprising a pharmaceutical product, drug, metabolite, or active ingredient.

The term “pharmaceutically-acceptable carrier” as used herein refers to one or more compatible solid or liquid filler, diluents or encapsulating substances which are suitable for administration to a human or other vertebrate animal.

The term “pharmaceutically acceptable salt” as used herein refers to those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002).

The phrase “powder for constitution” refers to powder blends containing the active ingredients and suitable diluents which can be suspended in water or juices.

The term “racemate” as used herein refers to an equimolar mixture of two optically active components that neutralize the optical effect of each other and is therefore optically inactive.

The term “reduce” or “reducing” as used herein refers to limit occurrence of a disorder in individuals at risk of developing the disorder.

The term “relative configuration” refers to the configuration of any stereogenic (asymmetric) center with respect to any other stereogenic center contained within the same molecular entity. Unlike absolute configuration, relative configuration is reflection-invariant. Relative configuration, distinguishing diastereoisomers may be denoted by the configurational descriptors R*,R* (or l) and R*,S* (or u) meaning, respectively, that the two centers have identical or opposite configurations. For molecules with more than two asymmetric centers, the prefix rel- may be used in front of the name of one enantiomer where R and S have been used. If any centers have known absolute configuration then only R* and S* can be used for the relative configuration. For example, two different molecules Xabcd and Xabce may be said to have the same relative configurations if e takes the position of d in the tetrahedral arrangement of ligands around X (i.e., the pyramidal fragments Xabc are superposable). Similarly, the enantiomer of Xabce may be said to have the opposite relative configuration to Xabcd. The terms may be applied to chiral molecular entities with central atoms other than carbon but are limited to cases where the two related molecules differ in a single ligand. These definitions can be generalized to include stereogenic units other than asymmetric centers.

The term “selective inhibitor” as used herein refers to an inhibitor showing measurable preference for binding to a given HDAC isoform over binding to other isoforms in order to achieve inhibition of histone deacetylase activity, as reflected by at least one order of magnitude difference in binding or inhibition activity obtained with the isoform to which the inhibitor is selective as compared to binding or inhibition activity obtained with other isoforms, respectively.

The term “stereogenic unit” (or “stereogen” or “stereoelement”) refers to a grouping within a molecular entity that may be considered a focus of stereoisomerism. At least one of these must be present in every enantiomer (though the presence of stereogenic units does not conversely require the corresponding chemical species to be chiral). Three basic types are recognized for molecular entities involving atoms having not more than four substituents: (a) a grouping of atoms consisting of a central atom and distinguishable ligands, such that the interchange of any two of the substituents leads to a stereoisomer. An asymmetric atom (chirality center) is the traditional example of this stereogenic unit; (b) a chain of four non-coplanar atoms (or rigid groups) in a stable conformation, such that an imaginary or real (restricted) rotation (with a change of sign of the torsion angle) about the central bond leads to a stereoisomer; and (c) a grouping of atoms consisting of a double bond with substituents which give rise to cis-trans isomerism.

The terms “subject” or “individual” or “patient” are used interchangeably to refer to a member of an animal species of mammalian origin, including humans.

The term “substituted” as used herein refers to replacement of an atom or a group of atoms by another as a result of a chemical reaction, multiple degrees of substitution being allowed unless otherwise stated.

The term “sulfanyl” as used herein refers to the substituent —S—.

The term “sulfenyl” as used herein refers to the substituent —S(O)—.

The term “sulfonyl” as used herein refers to the substituent —S(O)₂—.

The term “sustained release” (also referred to as “extended release”) is used herein in its conventional sense to refer to a drug formulation that provides for gradual release of a drug over an extended period of time, and that preferably, although not necessarily, results in substantially constant blood levels of a drug over an extended time period.

The term “syndrome,” as used herein, refers to a pattern of symptoms indicative of some disease or condition.

The term “symptom” as used herein refers to a phenomenon that arises from and accompanies a particular disease or disorder and serves as an indication of it.

The term “tablet” refers to a compressed or molded solid dosage form containing the active ingredients with suitable diluents. The tablet can be prepared by compression of mixtures or granulations obtained by wet granulation, dry granulation or by compaction.

The term “therapeutic agent” as used herein refers to a drug, molecule, nucleic acid, protein, metabolite, composition or other substance that provides a therapeutic effect. The terms “therapeutic agent” and “active agent” are used interchangeably herein. The active agent may be, for example, but not limited to, at least one of a compound of Formula I, Formula Ia, Formula Ib, Formula Ic, or a pharmaceutically acceptable salt thereof.

The term “therapeutically effective amount” refers to the amount necessary or sufficient to realize a desired biologic effect. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen may be planned which does not cause substantial toxicity and yet is effective to treat the particular subject. The effective amount for any particular application may vary depending on such factors as the disease or condition being treated, the particular inventive compound, the size of the subject, or the severity of the disease or condition. One of ordinary skill in the art may determine empirically the therapeutically effective amount of a particular inventive compound and/or other therapeutic agent without necessitating undue experimentation. It is generally preferred that a maximum dose be used, that is, the highest safe dose according to some medical judgment. The terms “dose” and “dosage” are used interchangeably herein.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50, which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation.

The term “topical” refers to administration of a composition at, or immediately beneath, the point of application. The phrase “topically applying” describes application onto one or more surfaces(s) including epithelial surfaces. Although topical administration, in contrast to transdermal administration, generally provides a local rather than a systemic effect, the terms “topical administration” and “transdermal administration” as used herein, unless otherwise stated or implied, are used interchangeably.

The term “treat” or “treating” as used herein refers to accomplishing one or more of the following: (a) reducing the severity of a disorder; (b) limiting development of symptoms characteristic of the disorder(s) being treated; (c) limiting worsening of symptoms characteristic of the disorder(s) being treated; (d) limiting recurrence of the disorder(s) in subjects that have previously had the disorder(s); and (e) limiting recurrence of symptoms in subjects that were previously symptomatic for the disorder(s).

The term “vitamin” as used herein, refers to any of various organic substances essential in minute quantities to the nutrition of most animals act especially as coenzymes and precursors of coenzymes in the regulation of metabolic processes.

### DETAILED DESCRIPTION

The described invention relates to novel histone deacetylase isoform-6 selective inhibitors, pharmaceutical compositions containing at least one such inhibitor, methods of preparing such inhibitors, and methods of using such inhibitors to treat HDAC-associated disorders.

### HDAC6-Selective Inhibitors

According to one aspect, the present invention provides compounds of Formula I:

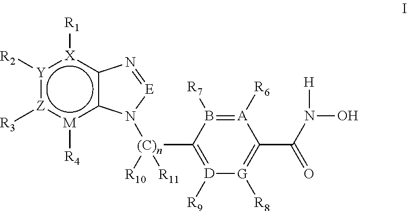

or a pharmaceutically acceptable salt thereof, wherein:

each of R₁, R₂, R₃ and R₄ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₁, R₂, R₃ and R₄ is H or a substituent when X, Y, Z and M is carbon;

E is C—R₅, or N;

R₅ is H, OH, NH₂, amino optionally substituted by alkyl or aryl, CN, F, Cl, Br, I, C₁-C₆ perfluoroalkyl, O-alkyl, O-aryl, O-heteroaryl, NO₂, cycloalkyl, aryl, acyl, optionally substituted C₁-C₆ alkyl, C₂-C₆ alkene, or C₂-C₆ alkyne, wherein when R₅ is OH, the compound exists as a keto tautomer, as an enol tautomer or as a mixture of keto-enol tautomers;

each of A, B, D, and G is independently C or N;

each of R₆, R₇, R₈, and R₉ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₆, R₇, R₈ and R₉ is H or a substituent when A, B, D and G is carbon;

each of R₁₀ and R₁₁ is independently H, alkyl, or aryl, wherein (C)ₙ optionally is a chiral center, wherein (C)ₙ can exist as both R and S enantiomers, with the proviso that when R₁₀ is H, R₁₁ is alkyl or aryl; and when R₁₁ is H, R₁₀ is alkyl or aryl; and
n is 0, 1, 2, 3, 4, 5, 6, 7, 8, 9 or 10.

wherein (C)ₙ can be monovalent, straight (unbranched) or branched hydrocarbon chain having 1 to 10 carbon atoms, saturated or unsaturated, wherein a double bond, if it exists, can be unconjugated or conjugated to another unsaturated group (e.g., a polyunsaturated alkenyl), can be unsubstituted or substituted, with multiple degrees of substitution being allowed, and can be optionally substituted with substituents selected from the group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, silyloxy optionally substituted by alkoxy, alkyl, or aryl, silyl optionally substituted by alkoxy, alkyl, or aryl, nitro, cyano, halogen, or lower perfluoroalkyl, with multiple degrees of substitution being allowed. Such an “alkenyl” group may contain one or more O, S, S(O), or S(O)₂ atoms. For example, and without limitation, the alkenyl can be vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, decenyl, undecenyl, dodecenyl, heptadecenyl, octadecenyl, nonadecenyl, eicosenyl, heneicosenyl, docosenyl, tricosenyl, tetracisenyl, pentacosenyl, phytyl, the branched chain isomers thereof, and polyunsaturated alkenes including octadec-9,12,-dienyl, octadec-9,12,15-trienyl, and eicos-5,8,11,14-tetraenyl.

According to some embodiments, the present invention provides compounds of Formula Ia:

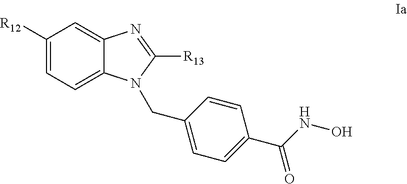

or a pharmaceutically acceptable salt thereof, wherein:

R₁₂ is H, alkyl, F, Cl, Br, I, or O-alkyl; and

R₁₃ is H or C₁-C₆ perfluoroalkyl.

According to some embodiments, the present invention provides compounds of Formula Ib:

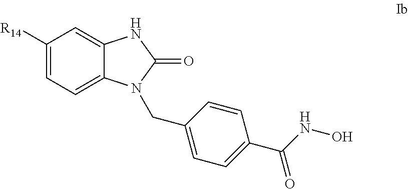

or a pharmaceutically acceptable salt thereof, wherein:

R₁₄ is H, alkyl, F, Cl, Br, I, O-alkyl, or C₁-C₆ perfluoroalkyl.

According to some embodiments, the present invention provides compounds of Formula Ic:

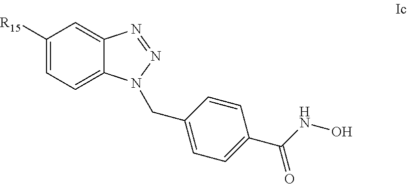

or a pharmaceutically acceptable salt thereof, wherein:

R₁₅ is H, alkyl, F, Cl, Br, I, or O-alkyl.

Particular embodiments of compounds of the present invention with different parts (moieties) or groups are discussed in more detail below. Those of ordinary skill in the art will appreciate that, unless otherwise indicated, each embodiment of each individual part or group may be independently combined with each embodiment of each other individual part or group in compounds of the present invention.

According to some embodiments, each of X, Y, Z and M, independently is C or N. According to some embodiments, each of X, Y, Z and M, independently is C. According to some embodiments, each of X, Y, Z and M, independently is N. According to some embodiments, X is C or N. According to some embodiments, X is C. According to some embodiments, X is N. According to some embodiments, Y is C or N. According to some embodiments, Y is C. According to some embodiments, Y is N. According to some embodiments, Z is C or N. According to some embodiments, Z is C. According to some embodiments, Z is N. According to some embodiments, M is C or N. According to some embodiments, Z is C. According to some embodiments, Z is N.

According to some embodiments, each of R₁, R₂, R₃ and R₄ are independently is H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₁, R₂, R₃ and R₄ is H or a substituent when X, Y, Z and M is carbon. According to some embodiments, each of R₁, R₂, R₃ and R₄ is independently OH. According to some embodiments, each of R₁, R₂, R₃ and R₄ is independently optionally substituted amino According to some embodiments, each of R₁, R₂, R₃ and R₄ is independently CN. According to some embodiments, each of R₁, R₂, R₃ and R₄ is independently F. According to some embodiments, each of R₁, R₂, R₃ and R₄ is independently Cl. According to some embodiments, each of R₁, R₂, R₃ and R₄ is independently Br. According to some embodiments, each of R₁, R₂, R₃ and R₄ is independently I. According to some embodiments, each of R₁, R₂, R₃ and R₄ is independently C₁-C₆ perfluoroalkyl. According to some embodiments, each of R₁, R₂, R₃ and R₄ is independently O-alkyl. According to some embodiments, each of R₁, R₂, R₃ and R₄ is independently O-aryl. According to some embodiments, each of R₁, R₂, R₃ and R₄ is independently O-heteroaryl. According to some embodiments, each of R₁, R₂, R₃ and R₄ is independently NO₂. According to some embodiments, each of R₁, R₂, R₃ and R₄ each of independently cycloalkyl. According to some embodiments, each of R₁, R₂, R₃ and R₄ each of independently aryl. According to some embodiments, each of R₁, R₂, R₃ and R₄ is independently acyl. According to some embodiments, each of R₁, R₂, R₃ and R₄ is independently optionally substituted C₁-C₆ alkyl. According to some embodiments, each of R₁, R₂, R₃ and R₄ is independently C₂-C₆ alkene. According to some embodiments, each of R₁, R₂, R₃ and R₄ is independently C₂-C₆ alkyne.

According to some embodiments, the compounds of the described invention are provided with the proviso that R₁, R₂, R₃ and R₄ is H or a substituent when X, Y, Z and M is carbon.

According to some embodiments, E is C—R₅ or N. According to some embodiments, E is C—R₅. According to some embodiments, E is N. According to some embodiments, E is C(O).

According to some embodiments, R₅ is H, OH, NH₂, amino optionally substituted by alkyl or aryl, CN, F, Cl, Br, I, C₁-C₆ perfluoroalkyl, O-alkyl, O-aryl, O-heteroaryl, NO₂, cycloalkyl, aryl, acyl, optionally substituted C₁-C₆ alkyl, C₂-C₆ alkene, or C₂-C₆ alkyne, wherein when R₅ is OH, the compound may exist as a keto tautomer, as an enol tautomer or as a mixture of keto-enol tautomers. According to some embodiments, R₅ is H. According to some embodiments, R₅ is OH. According to some embodiments, R₅ is optionally substituted amino. According to some embodiments, R₅ is CN. According to some embodiments, R₅ is F. According to some embodiments, R₅ is Cl. According to some embodiments, R₅ is Br. According to some embodiments, R₅ is I. According to some embodiments, R₅ is C₁-C₆ perfluoroalkyl. According to some embodiments, R₅ is O-alkyl. According to some embodiments, R₅ is O-aryl. According to some embodiments, R₅ is O-heteroaryl. According to some embodiments, R₅ is NO₂. According to some embodiments, R₅ is cycloalkyl. According to some embodiments, R₅ is aryl. According to some embodiments, R₅ is acyl. According to some embodiments, R₅ is optionally substituted C₁-C₆ alkyl. According to some embodiments, R₅ is C₂-C₆ alkene. According to some embodiments, R₅ is C₂-C₆ alkyne. According to some embodiments, compounds of the present invention may exist as a keto tautomer, as an enol tautomer or as a mixture of keto-enol tautomers. According to some embodiments, R₅ is C₂-C₆ alkyne. According to some embodiments, compounds of the present invention may exist as a keto tautomer. According to some embodiments, R₅ is C₂-C₆ alkyne. According to some embodiments, compounds of the present invention may exist as an enol tautomer. According to some embodiments, R₅ is C₂-C₆ alkyne. According to some embodiments, compounds of the present invention may exist as a mixture of keto-enol tautomers.

According to some embodiments, each of A, B, D, and G is independently C or N. According to some embodiments, each of A, B, D, and G is independently C. According to some embodiments, each of A, B, D, and G is independently N. According to some embodiments, A is C or N. According to some embodiments, A is C. According to some embodiments, A is N. According to some embodiments, B is C or N. According to some embodiments, B is C. According to some embodiments, B is N. According to some embodiments, D is C or N. According to some embodiments, D is C. According to some embodiments, D is N. According to some embodiments, G is C or N. According to some embodiments, G is C. According to some embodiments, G is N.

According to some embodiments, each of R₆, R₇, R₈, and R₉ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne; with the-proviso that R₁, R₂, R₃ and R₄ can be H or a substituent only when X, Y, Z and M is carbon. According to some embodiments, each of R₆, R₇, R₈, and R₉ is independently H. According to some embodiments, each of R₆, R₇, R₈, and R₉ is independently OH. According to some embodiments, each of R₆, R₇, R₈, and R₉ is independently optionally substituted amino According to some embodiments, each of R₆, R₇, R₈, and R₉ is independently CN. According to some embodiments, each of R₆, R₇, R₈, and R₉ is independently F. According to some embodiments, each of R₆, R₇, R₈, and R₉ is independently Cl. According to some embodiments, each of R₆, R₇, R₈, and R₉ is independently Br. According to some embodiments, each of R₆, R₇, R₈, and R₉ is independently I. According to some embodiments, each of R₆, R₇, R₈, and R₉ is independently C₁-C₆ perfluoroalkyl. According to some embodiments, R₆, R₇, R₈, and R₉ are each independently O-alkyl. According to some embodiments, each of R₆, R₇, R₈, and R₉ is independently O-aryl. According to some embodiments, each of R₆, R₇, R₈, and R₉ is independently O-heteroaryl. According to some embodiments, each of R₆, R₇, R₈, and R₉ is independently NO₂. According to some embodiments, each of R₆, R₇, R₈, and R₉ are is cycloalkyl. According to some embodiments, each of R₆, R₇, R₈, and R₉ is independently aryl. According to some embodiments, each of R₆, R₇, R₈, and R₉ is independently acyl. According to some embodiments, each of R₆, R₇, R₈, and R₉ is independently optionally substituted C₁-C₆ alkyl. According to some embodiments, each of R₆, R₇, R₈, and R₉ is independently C₂-C₆ alkene. According to some embodiments, each of R₆, R₇, R₈, and R₉ is independently C₂-C₆ alkyne.

According to some embodiments, each of R₁₀ and R₁₁ is independently H, alkyl, or aryl. According to some embodiments, compounds are provided with the proviso that when R₁₀ is H, R₁₁ is alkyl or aryl; and when R₁₁ is H, R₁₀ is alkyl or aryl. According to some embodiments, each of R₁₀ and R₁₁ is independently H. According to some embodiments, each of R₁₀ and R₁₁ is independently alkyl. According to some embodiments, each of R₁₀ and R₁₁ is independently aryl. According to some embodiments, (C)ₙ is optionally a chiral center, wherein (C)ₙ can exist as both R and S enantiomers.

According to some embodiments, compounds of the present invention are provided with the proviso that when R₁₀ is H, R₁₁ is alkyl or aryl; and when R₁₁ is H, R₁₀ is alkyl or aryl. According to some embodiments, compounds of the present invention are provided with the proviso that when R₁₀ is H, R₁₁ is alkyl or aryl. According to some embodiments, compounds of the present invention are provided with the proviso that when R₁₁ is H, R₁₀ is alkyl or aryl. According to some embodiments, compounds of the present invention are provided with the proviso that when R₁₁ is H, R₁₀ is H. According to some embodiments, compounds of the present invention are provided with the proviso that when R₁₁ is H, R₁₀ is alkyl. According to some embodiments, compounds of the present invention are provided with the proviso that when R₁₁ is H, R₁₀ is aryl. According to some embodiments, compounds of the present invention are provided with the proviso that when R₁₀ is H, R₁₁ is alkyl or aryl. According to some embodiments, compounds of the present invention are provided with the proviso that when R₁₀ is H, R₁₁ is H. According to some embodiments, compounds of the present invention are provided with the proviso that when R₁₀ is H, R₁₁ is alkyl. According to some embodiments, compounds of the present invention are provided with the proviso that when R₁₀ is H, R₁₁ is aryl.

According to some embodiments, n is 0, 1, 2, 3, 4, 5, 6, 7, 8, 9, or 10. According to some embodiments, n is 0. According to some embodiments, n is 1. According to some embodiments, n is 2. According to some embodiments, n is 3. According to some embodiments, n is 4. According to some embodiments, n is 5. According to some embodiments, n is 6. According to some embodiments, n is 7. According to some embodiments, n is 8. According to some embodiments, n is 9. According to some embodiments, n is 10.

According to some embodiments, a compound of Formula I comprises a metal binding moiety, a linker moiety, and a capping moiety.

According to some embodiments, the metal binding moiety is:

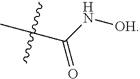 [SMILES: *C(=O)NO]

According to some embodiments, the linker moiety is:

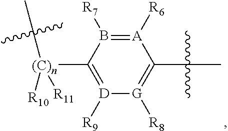

wherein:

each of A, B, D, and G is independently C or N;

each of R₆, R₇, R₈, and R₉ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy optionally substituted C₁-C₆ alkyl, C₂-C₆ alkene, or C₂-C₆ alkyne, with the-proviso that R₆, R₇, R₈ and R₉ is H or a substituent when A, B, D and G is carbon;

each of R₁₀ and R₁₁ is independently H, alkyl, or aryl, wherein (C)ₙ optionally is a chiral center, wherein (C)ₙ can exist as both R and S enantiomers, with the proviso that when R₁₀ is H, R₁₁ is alkyl or aryl; and when R₁₁ is H, R₁₀ is alkyl or aryl; and

n is an integer 0, 1, 2, 3, 4, 6, 7, 8, 9, or 10.

According to some embodiments, the linker moiety is:

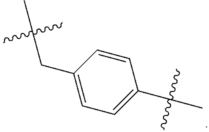 [SMILES: *Cc1ccc(*)cc1]

According to some embodiments, the capping moiety is:

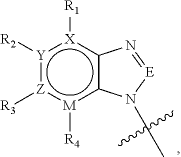

wherein:

each of X, Y, Z and M is independently C or N;

each of R₁, R₂, R₃ and R₄ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₁, R₂, R₃ and R₄ is H or a substituent when X, Y, Z and M is carbon; and

E is C—R₅, or N.

According to some embodiments, the capping moiety is selected from the group consisting of a benzimidazole, a benzimidazolone, and a benzitriazole.

According to some embodiments, the benzimidazole is:

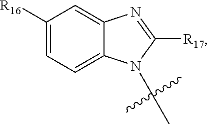

wherein:

each of R₁₆, and R₁₇ is independently H, OH, optionally substituted amino, CN, F, Br, I, C₁-C₆ perfluoroalkyl, O-alkyl, O-aryl, O-heteroaryl, NO₂, cycloalkyl, aryl, acyl, optionally substituted C₁-C₆ alkyl, C₂-C₆ alkene, or C₂-C₆ alkyne.

According to some embodiments, the benzimidazolone is:

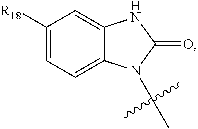

wherein:
R₁₈ is H, OH, optionally substituted amino, CN, F, Cl, Br, I, C₁-C₆ perfluoroalkyl, O-alkyl, O-aryl, O-heteroaryl, NO₂, cycloalkyl, aryl, acyl, optionally substituted C₁-C₆ alkyl, C₂-C₆ alkene, or C₂-C₆ alkyne.

According to some embodiments, the benzitriazole is:

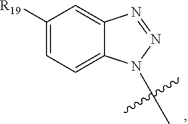

wherein:

R₁₉ is H, OH, optionally substituted amino, CN, F, Cl, Br, I, C₁-C₆ perfluoroalkyl, O-alkyl, O-aryl, O-heteroaryl, NO₂, cycloalkyl, aryl, acyl, optionally substituted C₁-C₆ alkyl, C₂-C₆ alkene, or C₂-C₆ alkyne.

### Stereochemistry

According to some embodiments, the compounds of the present invention have one or more chirality centers. According to some embodiments, the stereochemistry of the chiral centers represents all possible combinations in terms of relative and absolute chemistry. Accordingly, it may represent either racemic enantiomers or pure enantiomers.

Enantiomers show different properties (physical or chemical) only in a chiral medium. Polarized light provides such a medium, and in it enantiomers differ in a physical property: direction of the rotation of the light. They also may differ in solubility in an optically active solvent, or in adsorption on an optically active surface. For enantiomers to react at different rates, the necessary chiral medium can be provided in a number of ways: by an optically active reagent; by a chiral solvent, or the chiral surface of a catalyst. The terms “optically active reagent” or “chiral reagent” refer to reaction under any chiral condition. The terms “optically inactive reagent” or “achiral reagent” refer to reaction in the absence of a chiral medium.

Each chiral center is labeled R or S according to a system by which its substituents are each designated a priority according to the Cahn Ingold Prelog priority rules (CIP), based on atomic number. If the center is oriented so that the lowest priority of the four is pointed away from a viewer, the viewer will see two possibilities: if the priority of the remaining three substituents decreases in clockwise direction, it is labeled R (for Rectus), if it decreases in counterclockwise direction, it is S (for Sinister).

This system labels each chiral center in a molecule (and also has an extension to chiral molecules not involving chiral centers). Thus, it has greater generality than the D/L system, and can label, for example, an (R,R) isomer versus an (R,S)—diastereomers.

The R/S system has no fixed relation to the (+)/(−) system. An R isomer can be either dextrorotatory or levorotatory, depending on its exact substituents.

The R/S system also has no fixed relation to the D/L system. For example, the side-chain one of serine contains a hydroxyl group, —OH. If a thiol group, —SH, were swapped in for it, the D/L labeling would, by its definition, not be affected by the substitution. But this substitution would invert the molecule's R/S labeling, because the CIP priority of CH₂OH is lower than that for CO₂H but the CIP priority of CH₂SH is higher than that for CO₂H.

For this reason, the D/L system remains in common use in certain areas of biochemistry, such as amino acid and carbohydrate chemistry, because it is convenient to have the same chiral label for all of the commonly occurring structures of a given type of structure in higher organisms. In the D/L system, they are nearly all consistent—naturally occurring amino acids are nearly all L, while naturally occurring carbohydrates are nearly all D. In the R/S system, they are mostly S, but there are some common exceptions.

Superposability refers to the ability to bring two particular stereochemical formulae (or models) into coincidence (or to be exactly superposable in space, and for the corresponding molecular entities or objects to become exact replicas of each other) by no more than translation and rigid rotation.

As described herein, compounds may comprise one or more chirality centers, and thus can exist in various stereoisomeric forms, e.g., enantiomers, diastereomers, or geometric isomers. Thus, inventive compounds and pharmaceutical compositions thereof may be in the form of a racemic compound, an individual enantiomer (e.g., enantiomerically pure), an individual diastereomer (e.g., diastereomerically pure), an individual geometric isomer (e.g., geometrically pure), or may be in the form of a mixture of stereoisomers. In certain embodiments, compounds of the present invention are racemic compounds. In certain embodiments, compounds of the present invention are enantioenriched compounds. In certain embodiments, compounds of the present invention are diasteriomerically enriched compounds. In certain embodiments, wherein one or more double bonds is present, compounds of the present invention may be geometrically enriched compounds. In certain embodiments, compounds of the present invention are provided such that 75% of the preparation is of the same enantiomer or diastereomer. In certain embodiments, compounds of the present invention are provided such that at least 80%, 90%, 95%, or 97.5% of the preparation is of the same enantiomer or diastereomer. In certain embodiments, compounds of the present invention are provided such the preparation consists of a single enantiomer or diastereomer to the limits of detection (i.e., is “enantiopure”).

It will be apparent to one skilled in the art that each chiral center in a provided compound can be present in an (R)-configuration or in an (S)-configuration. In addition, where stereoisomeric forms of provided compounds may exist, such forms may be present in any ratio relative to one another. One skilled in the art will further understand that ratios of stereoisomers may vary according to methods by which such compounds are prepared. Exemplary ratios provided herein are meant to illustrate the present invention, and are not meant to limit the present invention.

With respect to geometric isomerism, the present invention contemplates both E and Z isomers wherein there exists one or more double bonds, unless otherwise indicated. According to some embodiments, the invention encompasses compounds as a single geometric isomer substantially free of other geometric isomers and alternatively, as mixtures of various isomers, e.g., racemic mixtures of E and Z isomers. In addition to the above-mentioned compounds per se, the invention also encompasses pharmaceutically acceptable derivatives of these compounds and compositions comprising one or more compounds of the invention and one or more pharmaceutically acceptable excipients or additives.

Where a stereoisomer is preferred, it may, According to some embodiments, be provided substantially free of other stereoisomers, as defined herein. According to certain embodiments, a compound of Formula I, Formula Ia, Formula Ib, Formula Ic, or a combination thereof is substantially free of other stereoisomers.

Enantiomeric and stereoisomeric mixtures may be resolved into their component enantiomers or stereoisomers by well known methods, such as chiral-phase gas chromatography, chiral-phase high performance liquid chromatography, crystallizing a compound as a chiral salt complex, or crystallizing a compound in a chiral solvent or by enzymatic resolution of a compound, its precursor or its derivative. Enantiomers and stereoisomers may also be obtained from stereomerically or enantiomerically pure intermediates, reagents, and catalysts by well-known asymmetric synthetic methods.

Unless otherwise stated, all tautomeric forms of the compounds of the invention are within the scope of the invention.

According to some embodiments, the present invention provides any compound depicted in Table 2, below, or a pharmaceutically acceptable salt thereof.

TABLE 2
Exemplary Compounds

| Compound | Details |
|---|---|
| 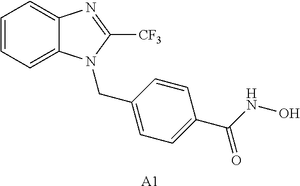 | Name: N-hydroxy-4-((2-(trifluoromethyl)-1H- benzo[d]imidazol-1-yl)methyl)benzamide Chemical Formula: C₁₆H₁₂F₃N₃O₂ Molecular Weight: 335.28 Data: ¹H NMR (400 MHz, DMSO-d₆) δ 5.74 (s, 2H), 7.12 (d, 2H), 7.41 (m, 2H), 7.67 (m, 2H), 7.86 (d, 2H); ¹³C NMR (400 MHz, s: DMSO) δ 47.90, 112.49, 121.46, 124.32, 126.37, 127.55, 136.05, 136.09, 138.48, 140.93, 140.96, 161.93; ESMS m/z 336.0 (M + 1) |
| 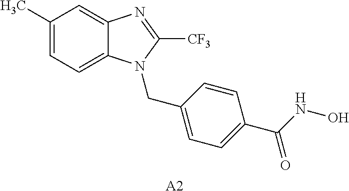 [SMILES: Cc1ccc2c(c1)nc(C(F)(F)F)n2Cc1ccc(C(=O)NO)cc1 ; "A2"] | Name: N-hydroxy-4-((5-methyl-2-(trifluoromethyl)-1H- benzo[d]imidazol-1-yl)methyl)benzamide Chemical Formula: C₁₇H₁₄F₃N₃O₂ Molecular Weight: 349.31 Data: ¹H NMR (400 MHz, DMSO-d₆) δ 2.42 (s, 3H), 5.7 (s, 2H), 7.07 (d, 2H), 7.25 (d, 1H), 7.50 (d, 1H), 7.63 (s, 1H), 7.67(m, 2H); ¹³C NMR (400 MHz, s: DMSO) δ: 21.48, 47.81, 111.98, 120.81, 126.43, 127.72, 127.79, 133.75, 134.19, 139.24, 139.69, 141.26, 163.94; ESMS m/z 350.1 (M + 1). |
| 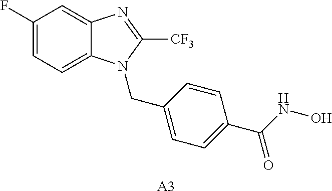 [SMILES: O=C(NO)c1ccc(Cn2c(C(F)(F)F)nc3cc(F)ccc32)cc1 ; "A3"] | Name: 4-((5-fluoro-2-(trifluoromethyl)-1H- benzo[d]imidazol-1-yl)methyl)-N-hydroxybenzamide Chemical Formula: C₁₆H₁₁F₄N₃O₂ Molecular Weight: 353.27 Data: ¹H NMR (400 MHz, DMSO-d₆) δ 5.76 (s, 2H), 7.12-7.14 (d, 2H), 7.34-7.36 (m, 1H), 7.68-7.70 (m, 4H), 9.03 (bs, 1H), 11.16 (bs, 1H); ¹³C NMR (400 MHz, s: DMSO) δ: 48.06, 106.70, 113.73, 113.83, 114.89, 115.15, 117.83, 126.55, 127.88, 132.84, 139.23, 141.15, 158.60, 160.97, 161.94, 164.13; ESMS m/z 354.0 (M + 1). |
| 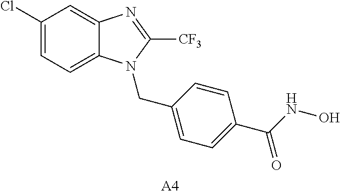 [SMILES: O=C(NO)c1ccc(Cn2c(C(F)(F)F)nc3cc(Cl)ccc32)cc1 ; "A4"] | Name: 4-((5-chloro-2-(trifluoromethyl)-1H- benzo[d]imidazol-1-yl)methyl)-N-hydroxybenzamide Chemical Formula: C₁₆H₁₁ClF₃N₃O₂ Molecular Weight: 369.73 Data: ¹H NMR (400 MHz, DMSO-d₆) δ 5.62 (s, 2H), 7.16 (d, 2H), 7.5 (d, 1H), 7.69 (m, 3H), 7.90 (s, 1H); ¹³C NMR (400 MHz, s: DMSO) δ: 48.07, 114.14, 118.50, 120.93, 126.53, 126.90, 127.82, 128.85, 134.93, 141.65, 145.19, 161.94; ESMS m/z 370.0 (M + 1). |
| 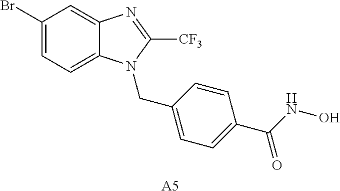 [SMILES: O=C(NO)c1ccc(Cn2c(C(F)(F)F)nc3cc(Br)ccc32)cc1 ; "A5"] | Name: 4-((5-bromo-2-(trifluoromethyl)-1H- benzo[d]imidazol-l-yl)methyl)-N-hydroxybenzamide Chemical Formula: C₁₆H₁₁BrF₃N₃O₂ Molecular Weight: 414.18 Data: ¹H NMR (400 MHz, DMSO-d₆) δ 5.76 (s, 2H), 7.12 (d, 2H), 7.58 (d, 1H), 7.69 (d, 3H), 8.11 (s, 1H); ¹³C NMR (400 MHz, s: DMSO) δ: 48.07, 114.50, 116.63, 117.76, 120.47, 123.95, 126.54, 127.85, 129.08, 135.23, 139.07, 141.01, 141.39, 142.17, 161.94, 164.01; ESMS m/z 414.9 (M + 1). |
| 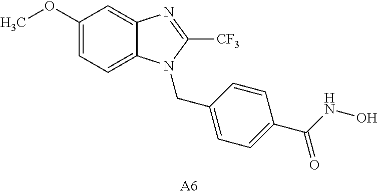 [SMILES: COc1ccc2c(c1)nc(C(F)(F)F)n2Cc1ccc(C(=O)NO)cc1 ; "A6"] | Name: N-hydroxy-4-((5-methoxy-2-(trifluoromethyl)-1H- benzo[d]imidazol-1-yl)methyl)benzamide Chemical Formula: C₁₇H₁₄F₃N₃O₃ Molecular Weight: 365.31 Data: ¹H NMR (400 MHz, DMSO-d₆) δ3.79 (s, 3H), 5.74 (s, 2H), 7.10-7.67 (m, 7H), 9.02 (s, 1H), 11.16 (s, 1H); ¹³C NMR (400 MHz, s: DMSO) δ: 47.74, 48.90, 55.86, 102.57, 112.77, 116.72, 117.98, 120.68, 127.75, 130.44, 132.63, 139.45, 141.77, 157.17, 164.27; ESMS m/z 366.0 (M + 1). |
| 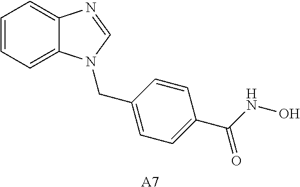 | Name: 4-((1H-benzo[d]imidazol-1-yl(methyl)-N- hydroxybenzamide Chemical Formula: C₁₅H₁₃N₃O₂ Molecular Weight: 267.28 Data: ¹H NMR (400 MHz, DMSO-d₆) δ 5.54 (s, 2H), 7.18 (m, 2H), 7.34 (d, 2H), 7.48 (m, 1H), 7.68 (m. 3H), 8.42 (s, 1H); ¹³C NMR (400 MHz, s: DMSO) δ: 47.74, 111.12, 119.98, 122.14, 122.95, 127.75, 132.68, 134.04, 140.43, 143.97, 144.75, 164.24; ESMS m/z 268.2 (M + 1). |
| 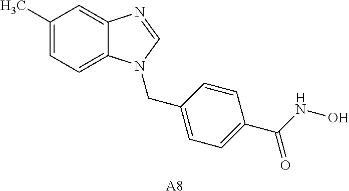 [SMILES: Cc1ccc2c(c1)ncn2Cc1ccc(C(=O)NO)cc1 ; "A8"] | Name: N-hydroxy-4-((5-methyl-1H-benzo[d]imidazol-1- yl)methyl)benzamide Chemical Formula: C₁₆H₁₅N₃O₂ Molecular Weight: 281.31 Data: ¹H NMR (400 MHz, DMSO-d₆) δ 2.35 (s, 3H), 5.49 (s, 2H), 7.01 (d, 1H), 7.31 (m, 3H), 7.43 (s, 1H), 7.69 (m, 2H), 8.31 (s, 1H); ¹³C NMR (400 MHz, s: DMSO) δ: 21.54, 47.74, 110.65, 119.70, 124.36, 127.67, 127.70, 131.19, 132.17, 132.65, 140.53, 144.37, 144.65, 164.22; ESMS m/z 282.2 (M + 1). |
| 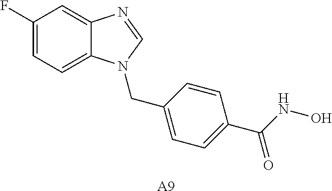 [SMILES: O=C(NO)c1ccc(Cn2cnc3cc(F)ccc32)cc1 ; "A9"] | Name: 4-((5-fluoro-1H-benzo[d]imidazol-1-yl)methyl)-N- hydroxybenzamide Chemical Formula: C₁₅H₁₂FN₃O₂ Molecular Weight: 285.27 Data: ¹H NMR (400 MHz, DMSO-d₆) δ 5.54 (s, 2H), 7.07 (m, 1H), 7.34 (d, 2H), 7.48 (m, 2H), 7.69 (d, 2H), 8.49 (bs, 1H), 9.04 (s. 1H), 11.18 (s, 1H); ¹³C NMR (400 MHz, s: DMSO) δ 47.91, 105.39, 111.27, 111.99, 130.77, 132.76, 140.21, 144.29, 146.41, 157.78, 160.12, 161.93, 164.22; ESMS m/z 286.4 (M + 1). |
| 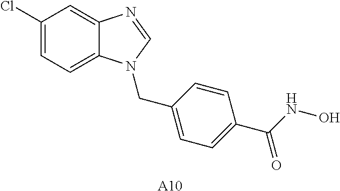 [SMILES: O=C(NO)c1ccc(Cn2cnc3cc(Cl)ccc32)cc1 ; "A10"] | Name: 4-((5-chloro-1H-benzo[d]imidazol-1-yl)methyl)-N- hydroxybenzamide Chemical Formula: C₁₅H₁₂ClN₃O₂ Molecular Weight: 301.73 Data: ¹H NMR (400 MHz, DMSO-d₆) δ 5.56 (s, 2H), 7.04-8.05 (m, 7H), 8.51 (bs, 1H), 9.05 (s, 1H), 11.18 (s. 1H); ¹³C NMR (400 MHz, DMSO) δ: 47.90, 112.58, 119.47, 123.14, 126.79, 127.76, 132.77, 132.90, 140.11, 144.88, 146.32, 164.18, 164.20; ESMS m/z 302.0 (M + 1). |
| 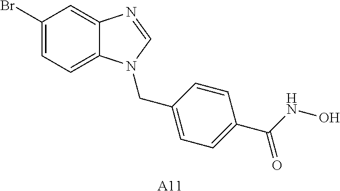 [SMILES: O=C(NO)c1ccc(Cn2cnc3cc(Br)ccc32)cc1 ; "A11"] | Name: 4-((5-bromo-1H-benzo[d]imidazol-1-yl)methyl)-N- hydroxybenzamide Chemical Formula: C₁₅H₁₂BrN₃O₂ Molecular Weight: 346.18 Data: ¹H NMR (400 MHz, DMSO-d₆) δ 5.56 (s, 2H), 6.99-8.10 (m, 7H), 8.49 (s, 1H), 9.07 (bs, 1H), 11.19 (s. 1H); ¹³C NMR (400 MHz, DMSO) δ: 47.90, 113.07, 114.60, 122.46, 125.72, 127.75, 132.75, 140.07, 145.39, 146.22, 146.23, 164.16, 164.18; ESMS m/z 347.1 (M + 1). |
| 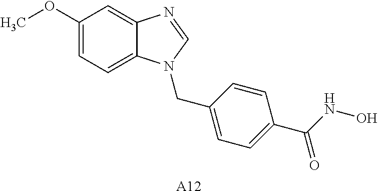 [SMILES: COc1ccc2c(c1)ncn2Cc1ccc(C(=O)NO)cc1 ; "A12"] | Name: N-hydroxy-4-((5-methoxy-1H-benzo[d]imidazol-1- yl)methyl)benzamide Chemical Formula: C₁₆H₁₅N₃O₃ Molecular Weight: 297.31 Data: ¹H NMR (DMSO-d6) δ 3.56 (s, 3H), 5.32 (s, 2H), 6.64 (d, 1H), 7.01 (s, 1H), 7.09-7.27 (m, 3H), 7.5 l (d, 2H), 8.23 (s, 1H), 11.02 (s, 1H); ¹³C NMR (400 MHz, DMSO) δ: 47.91, 55.91, 102.29, 111.58, 112.87, 127.72, 132.64, 140.44, 156.02, 164.36, 164.38, 164.40; ESMS m/z 298.5 (M + 1). |
| 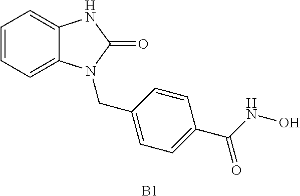 | Name: N-hydroxy-4-((2-oxo-2,3-dihydro-1H- benzo[d]imidazol-1-yl)methyl)benzamide Chemical Formula: C₁₅H₁₃N₃O₃ Molecular Weight: 283.28 Data: ¹HNMR (DMSO-d6) δ 5.04 (s, 2H), 6.95-6.99 (m, 4H), 7.35 (dd, 2H), 7.69 (dd, 2H), 10.98 (bs, 1H); ¹³C NMR (400 MHz, DMSO) δ: 43.40, 61.83, 101.43, 108.48, 109.37, 121.04, 121.58, 127.70, 128.74, 130.28, 130.32, 132.40, 141.19, 154.78, 164.4; ESMS m/z 284.1 (M + 1). |
| 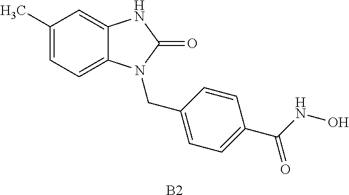 [SMILES: Cc1ccc2c(c1)[nH]c(=O)n2Cc1ccc(C(=O)NO)cc1 ; "B2"] | Name: N-hydroxy-4-((5-methyl-2-oxo-2,3-dihydro-1H- benzo[d]imidazol-1-yl)methyl)benzamide Chemical Formula: C₁₆H₁₅N₃O₃ Molecular Weight: 297.31 Data: ¹H NMR (400 MHz, DMSO-d₆) δ 2.24 (s, 3H), 4.98 (s, 2H), 6.71-6.85 (m, 3H), 7.30-7.32 (d, 2H), 7.66-7.68 (d, 2H), 10.89 (s, 1H); ¹³C NMR (400 MHz, DMSO) δ: 21.31, 43.29, 48.96, 108.13, 109.84, 121.48, 127.53, 127.56, 128.78, 130.69, 132.23, 140.78, 154.83, 164.34; ESMS m/z 298.1 (M + 1). |
| 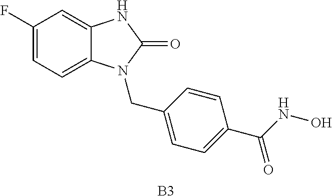 [SMILES: O=C(NO)c1ccc(Cn2c(=O)[nH]c3cc(F)ccc32)cc1 ; "B3"] | Name: 4-((5-fluoro-2-oxo-2,3-dihydro-1H- benzo[d]imidazol-1-yl)methyl)N-hydroxybenzamide Chemical Formula: C₁₅H₁₂FN₃O₃ Molecular Weight: 301.27 Data: ¹H NMR (400 MHz, DMSO-d₆) δ 5.02 (s, 2H), 6.74-6.99 (m, 3H), 7.32-7.34 (d, 2H), 7.67-7.69 (d, 2H), 11.12 (s, 1H); ¹³C NMR (400 MHz, DMSO) δ 43.39, 97.29, 97.58, 107.04, 108.78, 127.62, 129.31, 129.44, 132.38, 140.49, 155.03, 157.11, 159.44, 164.27; ESMS m/z 302.0 (M + 1). |
| 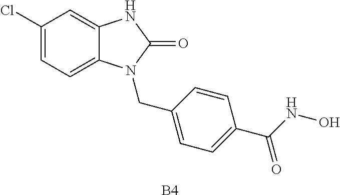 [SMILES: O=C(NO)c1ccc(Cn2c(=O)[nH]c3cc(Cl)ccc32)cc1 ; "B4"] | Name: 4-((5-chloro-2-oxo-2,3-dihydro-1H- benzo[d]imidazol-1-yl)methyl)-N-hydroxybenzamide Chemical Formula: C₁₅H₁₂ClN₃O₃ Molecular Weight: 317.73 Data: ¹H NMR (400 MHz, DMSO-d₆) δ 5.04 (s, 2H), 6.91-7.05 (m, 3H), 7.33 (d, 2H), 7.74 (d, 2H), 11.16 (s, 1H); ¹³C NMR (400 MHz, s: DMSO) δ: 43.47, 109.36, 109.62, 120.77, 125.76, 127.69, 127.40, 129.93, 129.80, 132.49, 140.42, 154.75, 164.33; ESMS m/z 318.0 (M + 1). |
| 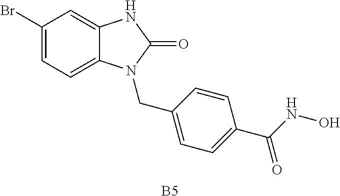 [SMILES: O=C(NO)c1ccc(Cn2c(=O)[nH]c3cc(Br)ccc32)cc1 ; "B5"] | Name: 4-((5-bromo-2-oxo-2,3-dihydro-1H- benzo[d]imidazol-1-yl)methyl)-N-hydroxybenzamide Chemical Formula: C₁₅H₁₂BrN₃O₃ Molecular Weight: 362.18 Data: ¹H NMR (400 MHz, DMSO-d₆) δ 5.02 (s, 2H), 6.95-6.97 (d, 1H), 7.19-7.13 (m, 2H), 7.31-7.33 (d, 2H), 7.66-7.68 (d, 2H), 9.0 (s, 1H), 11.16 (s, 1H); ¹³C NMR (400 MHz, s: DMSO) δ 43.46, 110.17, 112.02, 113.29, 123.58, 127.64, 129.66, 130.26, 132.45, 140.37, 154.59, 161.94, 164.32; ESMS m/z 363.0 (M + 1). |
| 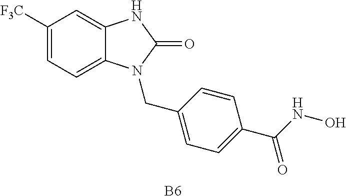 [SMILES: O=C(NO)c1ccc(Cn2c(=O)[nH]c3cc(C(F)(F)F)ccc32)cc1 ; "B6"] | Name: N-hydroxy-4-((2-oxo-5-(trifluoromethyl)-2,3- dihydro-1H-benzo[d]imidazol-1-yl)methyl)benzamide Chemical Formula: C₁₆H₁₂F₃N₃O₃ Molecular Weight: 351.28 Data: ¹H NMR (400 MHz, DMSO-d₆) δ 5.09 (s, 2H), 7.19-7.35 (m, 5H), 7.67-7.69 (d, 2H), 9.01 (s, 1H), 11.25 (s, 1H); ¹³C NMR (400 MHz, s: DMSO) δ 43.52, 105.85, 108.58, 118.44, 118.48, 121.95, 122.28, 127.59, 128.91, 132.46, 133.27, 140.12, 154.84, 164.24; ESMS m/z 352.0 (M + 1). |
| 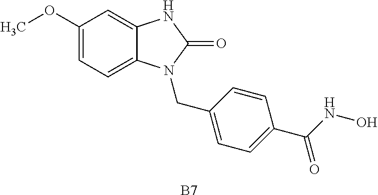 [SMILES: COc1ccc2c(c1)[nH]c(=O)n2Cc1ccc(C(=O)NO)cc1 ; "B7"] | Name: N-hydroxy-4-((5-methoxy-2-oxo-2,3-dihydro-1H- benzo[d]imidazol-1-yl)methyl)benzamide Chemical Formula: C₁₆H₁₅N₃O₄ Molecular Weight: 313.31 Data: ¹H NMR (400 MHz, DMSO-d₆) δ 3.647-3.71 (s, 3H), 4.98 (s, 2H), 6.50-6.58 (m, 2H), 6.85-6.87 (d, 1H), 7.31-7.33 (d, 2H), 7.67-7.74 (d, 2H), 9.01 (s, 1H), 10.89 (s, 1H), 11.15 (s, 1H); ¹³C NMR (400 MHz, s: DMSO) δ 43.52, 105.85, 108.58, 118.44, 121.97, 122.28, 127.67, 128.91, 132.46, 133.27, 133.28, 140.12, 154.84, 164.24; ESMS m/z 314.1 (M + 1). |
| 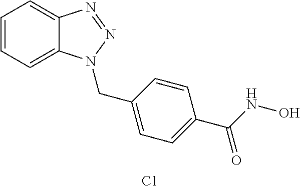 | Name: 4-((1H-benzo[d][1,2,3]triazol-1-yl)methyl)-N- hydroxybenzamide Chemical Formula: C₁₄H₁₂N₄O₂ Molecular Weight: 268.27 Data: ¹H NMR (DMSO-d6) δ 6.02 (s, 2H), 7.35-7.41 (m, 3H), 7.49-7.52 (m, 1H), 7.67-7.70 (m, 2H), 7.80-7.84 (d, 1H), 8.04-8.06 (d, 1H); ¹³C NMR (400 MHz, DMSO) δ: 50.98, 111.05, 119.71, 124.59, 127.83, 128.02, 128.12, 132.99, 133.14, 139.28, 145.75, 164.25; ESMS m/z 269.0 (M + 1). |
| 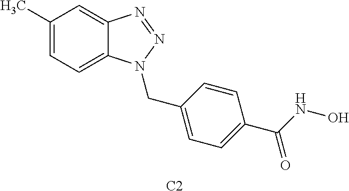 [SMILES: Cc1ccc2c(c1)nnn2Cc1ccc(C(=O)NO)cc1 ; "C2"] | Name: N-hydroxy-4-((5-methyl-1H-benzo[d][1,2,3]triazol-1- yl)methyl)benzamide Chemical Formula: C₁₅H₁₄N₄O₂ Molecular Weight: 282.30 Data: ¹H NMR (DMSO-d6) δ 2.43 (s, 3H), 5.97 (s, 2H), 7.31-7.35 (m, 3H), 7.65-7.71 (m, 3H), 7.80 (s, 1H); ¹³C NMR (400 MHz, DMSO) δ: 21.91, 51.02, 61.85, 101.37, 110.59, 118.45, 128.11, 129.28, 134.20, 146.35, 161.94, 164.12, 166.17; ESMS m/z 283.1 (M + 1). |
| 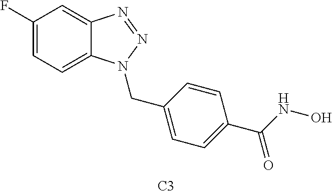 [SMILES: O=C(NO)c1ccc(Cn2nnc3cc(F)ccc32)cc1 ; "C3"] | Name: 4-((5-fluoro-1H-benzo[d][1,2,3]triazol-1-yl)methyl)- N-hydroxybenzamide Chemical Formula: C₁₄H₁₁FN₄O₂ Molecular Weight: 286.26 Data: ¹H NMR (DMSO-d6) δ 5.91 (s, 2H), 7.24-7.36 (d, 3H), 7.57-7.59 (m, 2H), 7.76-7.78 (m, 2H); ¹³CNMR (400 MHz, DMSO) δ: 51.23, 104.14, 104.38, 112.75, 117.70, 117.97, 127.85, 130.40, 133.07, 139.03, 145.82, 158.43, 160.82, 164.20; ESMS m/z 287.0 (M + 1). |
| 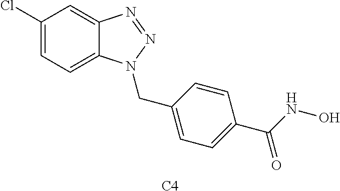 [SMILES: O=C(NO)c1ccc(Cn2nnc3cc(Cl)ccc32)cc1 ; "C4"] | Name: 4-((5-chloro-1H-benzo[d][1,2,3]triazol-1-yl)methyl)- N-hydroxybenzamide Chemical Formula: C₁₄H₁₁ClN₄O₂ Molecular Weight: 302.72 Data: ¹H NMR (DMSO-d6) δ 5.95 (s, 2H), 7.27-7.29 (m, 2H), 7.47-7.49 (d, 1H), 7.62-7.64 (m, 2H), 7.80-7.82 (d, 1H), 8.11 (s, 1H); ¹³CNMR (400 MHz, DMSO) δ: 51.22, 112.78, 119.02, 127.73, 127.86, 128.04, 129.28, 132.08, 133.07, 138.97, 146.37, 164.19; ESMS m/z 303.0 (M + 1). |
| 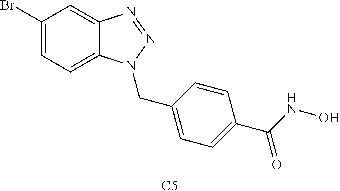 [SMILES: O=C(NO)c1ccc(Cn2nnc3cc(Br)ccc32)cc1 ; "C5"] | Name: 4-((5-bromo-1H-benzo[d][1,2,3]triazol-1-yl)methyl)- N-hydroxybenzamide Chemical Formula: C₁₄H₁₁BrN₄O₂ Molecular Weight: 347.17 Data: ¹H NMR (DMSO-d6) δ 6.03 (s, 2H), 7.30-7.32 (m, 2H), 7.63-7.65 (m, 2H), 7.78-7.79 (d, 1H), 8.01-8.03 (d, 1H), 8.48 (s, 1H); ¹³C NMR (400 MHz, DMSO) δ: 51.29, 112.86, 118.24, 124.43, 124.46, 127.90, 128.20, 134.83, 138.87, 144.87, 161.93, 164.22; ESMS m/z 348.0 (M + 1). |

According to some embodiments, a compound of Formula I, Formula Ia, Formula Ib, Formula Ic, or a combination thereof may be provided according to the present invention in any of a variety of useful forms, for example as pharmaceutically acceptable salts, as particular crystal forms, etc. According to some embodiments, a prodrug of one or more compounds of the present invention are provided. Various forms of prodrug are known in the art, for example as discussed in Bundgaard (ed.), Design of Prodrugs, Elsevier (1985); Widder et al. (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975).

According to some embodiments, provided compounds are considered inhibitors in that they inhibit the histone deacetylating activity of histone deacetylase enzymes, i.e., removal of acetyl groups from an acetylated ε-amino group of a conserved lysine residue on a histone. According to some embodiments, provided compounds are inhibitors of HDAC1, HDAC2, HDAC3, HDAC4, HDAC5, HDAC6, HDAC7, HDAC8, HDAC9, HDAC10, HDAC11, or a combination thereof. According to some embodiments, provided compounds are inhibitors of HDAC1. According to some embodiments, provided compounds are inhibitors of HDAC2. According to some embodiments, provided compounds are inhibitors of HDAC3. According to some embodiments, provided compounds are inhibitors of HDAC4. According to some embodiments, provided compounds are inhibitors of HDAC5. According to some embodiments, provided compounds are inhibitors of HDAC6. According to some embodiments, provided compounds are inhibitors of HDAC7. According to some embodiments, provided compounds are inhibitors of HDAC8. According to some embodiments, provided compounds are inhibitors of HDAC9. According to some embodiments, provided compounds are inhibitors of HDAC10. According to some embodiments, provided compounds are inhibitors of HDAC11. According to some embodiments, provided compounds are selective inhibitors of HDAC6.

According to some embodiments, the HDAC inhibitor inhibits the histone deacetylating activity of an HDAC isoform selected from the group consisting of HDAC1, HDAC2, HDAC3, HDAC4, HDAC5, HDAC7, HDAC8, HDAC9, HDAC10, HDAC11 with a histone deacetylase inhibition activity (IC₅₀) ranging from about 0.005 μM to about 3 μM. According to some embodiments, the HDAC inhibitor inhibits the histone deacetylating activity of an HDAC isoform selected from the group consisting of HDAC1, HDAC2, HDAC3, HDAC4, HDAC5, HDAC7, HDAC8, HDAC9, HDAC10, HDAC11 with a histone deacetylase inhibition activity (IC₅₀) of at least about 0.005 μM, at least about 0.010 μM, at least about 0.020 μM, at least about 0.030 μM, at least about 0.040 μM, at least about 0.050 μM, at least about 0.060 μM, at least about 0.070 μM, at least about 0.080 μM, at least about 0.090 μM, at least about 0.1 μM, at least about 0.2 μM, at least about 0.3 μM, at least about 0.4 μM, at least about 0.5 μM, at least about 0.6 μM, at least about 0.7 μM, at least about 0.8 μM, at least about 0.9 μM, at least about 1 μM, at least about 1.1 μM, at least about 1.2 μM, at least about 1.3 μM, at least about 1.4 μM, at least about 1.5 μM, at least about 1.6 μM, at least about 1.7 μM, at least about 1.8 μM, at least about 1.9 μM, at least about 2 μM, at least about 2.1 μM, at least about 2.2 μM, at least about 2.3 μM, at least about 2.4 μM, at least about 2.5 μM, at least about 2.6 μM, at least about 2.7 μM, at least about 2.8 μM, at least about 2.9 μM.

According to some embodiments, the HDAC inhibitor selectively inhibits the histone deacetylating activity of HDAC6. According to one embodiment, the HDAC inhibitor inhibits the histone deacetylating activity of HDAC6 with an inhibition activity (IC₅₀) ranging from about 0.000001 μM to about 0.001 μM. According to one embodiment, the histone deacetylase inhibition activity (IC₅₀) is at least about 0.000001 μM. According to another embodiment, the histone deacetylase inhibition activity (IC₅₀) is at least about 0.000005 μM. According to another embodiment, the histone deacetylase inhibition activity (IC₅₀) is at least about 0.00001 μM. According to another embodiment, the histone deacetylase inhibition activity (IC₅₀) is at least about 0.00005 μM. According to another embodiment, the histone deacetylase inhibition activity (IC₅₀) is at least about 0.0001 μM. According to another embodiment, the histone deacetylase inhibition activity (IC₅₀) is at least about 0.0005 μM. According to another embodiment, the histone deacetylase inhibition activity (IC₅₀) is about 0.001 μM.

### Methods of Synthesis

According to another aspect, the present invention provides methods of preparing compounds provided herein. As will be appreciated by one of skill in the art, the synthetic methods described herein may be modified without departing from the scope of the present invention. For example, different starting materials and/or different reagents may be used in the inventive synthetic methods.

According to one embodiment, the present invention provides a process for preparing a substituted benzimidazole as an HDAC inhibitor. In some such embodiments, the inventive compounds are prepared as shown in the scheme below:

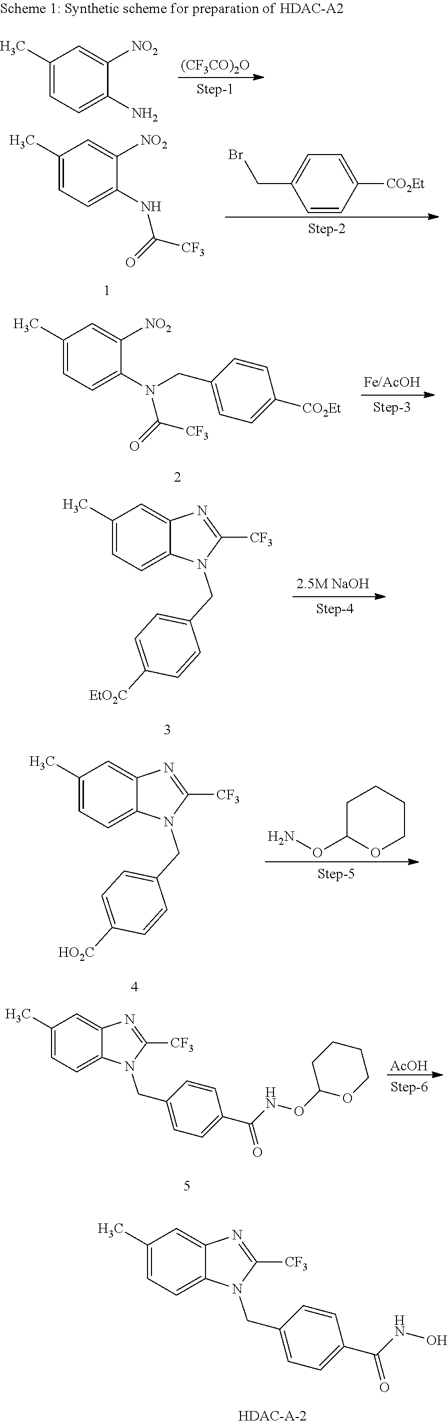

Step-1: A solution of 4-methyl-2-nitro aniline (10 g, 65.8 mmol, 1 eq) in dichloromethane (100 mL) was cooled to 0° C. and stirred for 30 minutes. Trifluoroacetic anhydride (18.3 mL, 131 mmol, 2 eq) was added and the reaction mixture was stirred at 0° C. for another 30 minutes. After completion of the reaction, NaHCO₃ was added to neutralize the reaction. The organic layer was separated and evaporated to dryness to yield compound 1 as a yellow solid (yield: 76%).

Step-2: To a solution of compound 1 (4.0 g, 16.1 mmol, 1 eq) in DMF (15 mL) was added Potassium carbonate (4.45 g, 32.3 mmol, 2 eq) and stirred at rt for 15 minutes. Ethyl 4-(bromomethyl)benzoate (4.43 g, 19.4 mmol, 1.1 eq) dissolved in DMF (5 mL) was added drop-wise and the resulting mixture was refluxed for 4 hrs at 50-60° C. After completion of the reaction, the reaction mixture was extracted using water and ethyl acetate and evaporated to dryness to yield compound 2 (yield: 95%).

Step-3: To a solution of compound 2 (1.0 g, 2.5 mmol, 1 eq) in AcOH (10 mL) and EtOH (10 mL) was added iron powder (1 g, 17.8 mmol, 7.12 eq) and refluxed for 3 hrs. After completion of the reaction, the reaction mixture was filtered and the filtrate was treated with water and extracted with EtOAc. The Organic layer was washed with aq. base and dried over anhydrous magnesium sulfate. The ethyl acetate layer was evaporated to dryness to yield compound 3 (yield: 72%). 1H NMR (DMSO-d6) δ 1.22-1.26 (t, 3H), 2.40 (s, 3H), 4.21-4.26 (m, 2H), 5.73 (s, 2H), 7.12-7.15 (dd, 2H), 7.22-7.24 (d, 1H), 7.48-7.50 (d, 1H), 7.62 (s, 1H), 7.85-7.87 (dd, 2H).

Step-4: To a solution of compound 3 (1.0 g, 2.7 mmol) in methanol (15 mL) was added 2.5 M NaOH (3 mL) and refluxed for 3 hrs. After completion of the reaction, methanol was removed by distillation and the reaction mixture was neutralized with acetic acid. The target compound was extracted with dichloromethane and evaporated to dryness to yield compound 4 (yield: 85%). ¹H NMR (DMSO-d6) δ 2.39 (s, 3H), 5.71 (s, 2H), 7.10-7.12 (dd, 2H), 7.21-7.23 (d, 1H), 7.48-7.50 (d, 1H), 7.61 (s, 1H), 7.84-7.86 (dd, 2H).

Step-5: To a solution of compound 4 (0.47 g, 1.4 mmol) in DMF (6 mL) and triethylamine (0.37 mL, 3 mmol, 2 eq) was added HATU (0.606 g, 1.6 mmol, 1.2 eq) in DMF (3 mL) and stirred for 15 minutes at room temperature. The O-(tetrahydro-2H-pyran-2-yl)-hydroxylamine (0.187 g, 1.6 mmol, 1.2 eq) in DMF (1 mL) was added to the first solution. The resulting solution was stirred at rt for 12 hrs. After completion of the reaction, water was added to the reaction mixture. The solid thus formed was filtered, dried and purified by washing with ether. The compound was used in the next step as it is without any further purification (yield: 66%).

Step-6: To a solution of compound 5 (0.2 g, 0.450 mmol) in THF (5 mL) was added AcOH (10 mL) and water (3 mL). The resulting solution was stirred at 60° C. for 6 hrs. After completion of the reaction, the solvents were evaporated in vacuum. The solid thus formed was washed with water, filtered, dried and purified by preparative TLC using 50% EtoAc in Hexane (R_{f}=0.36) to yield the target compound HDAC-A-2 (yield: 63%). ¹H NMR (400 MHz, DMSO-d₆) δ 2.42 (s, 3H), 5.7 (s, 2H), 7.07 (d, 2H), 7.25 (d, 1H), 7.50 (d, 1H), 7.63 (s, 1H), 7.67 (m, 2H); ¹³C NMR (400 MHz, s: DMSO) δ: 21.48, 47.81, 111.98, 120.81, 126.43, 127.72, 127.79, 133.75, 134.19, 139.24, 139.69, 141.26, 163.94; ESMS m/z 350.1 (M+1).

HDAC inhibitor compounds A1, A3, A4, A5, A6, A7, A8, A9, A10, A11, and A12 were synthesized by using the same synthetic scheme as given for HDAC inhibitor compound A2 using appropriate starting materials.

According to another embodiment, the present invention provides a process for preparing a substituted benzimidazolone as an HDAC inhibitor. In some such embodiments, the inventive compounds are prepared as shown in the scheme below:

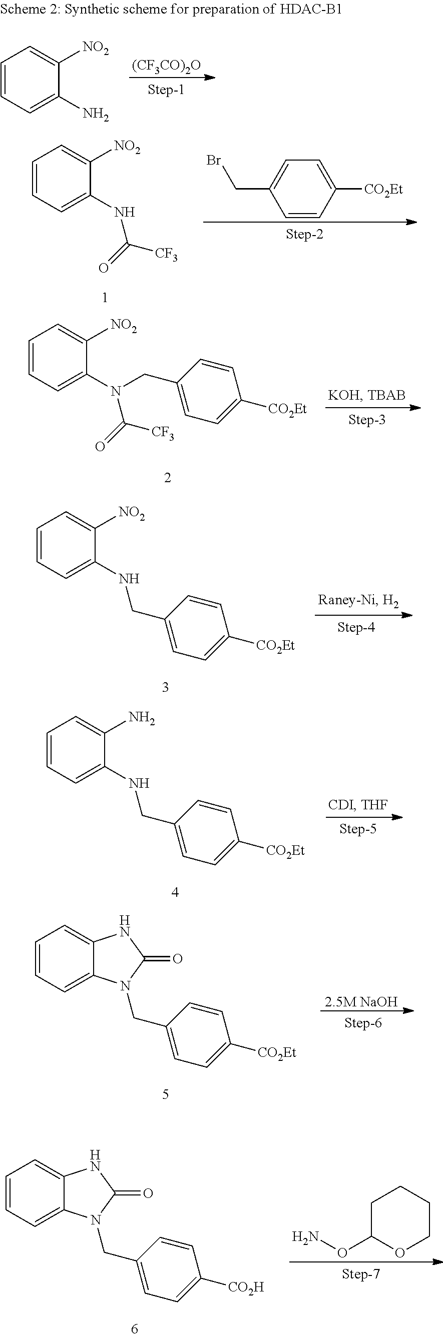

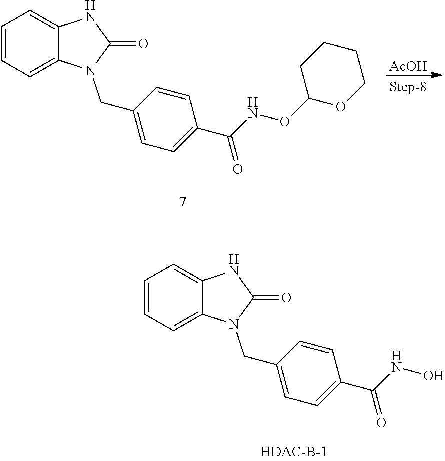 [SMILES: "AcOH" ; O=C(NOC1CCCCO1)c1ccc(Cn2c(=O)[nH]c3ccccc32)cc1 ; O=C(NO)c1ccc(Cn2c(=O)[nH]c3ccccc32)cc1]

Step-1: A solution of 2-nitro aniline (10 g, 50.7 mmol, 1 eq) in dichloromethane (100 mL) was cooled to 0° C. and stirred for 30 minutes. TFAA (14.1 mL, 101.4 mmol, 2 eq) was added and the reaction mixture was stirred at 0° C. for another 30 minutes. After completion of the reaction, NaHCO₃ was added to neutralize the reaction. The organic layer was separated and distilled to dryness to yield compound 1 as a yellow solid (yield: 82%). ¹H NMR (DMSO-d6) δ 7.55 (t, 1H), 7.72 (d, 1H), 7.75 (t, 1H), 7.97 (d, 1H), 11.6 (bs, 1H).

Step-2: To a solution of compound 1 (17.2 mmol, 1 eq) in DMF (15 mL) was added potassium carbonate (4.75 g, 34.4 mmol, 2 eq) and stirred at rt for 15 minutes. Ethyl 4-(bromomethyl)benzoate (4.32 g, 18.9 mmol, 1.1 eq) dissolved in DMF (5 mL) was added drop-wise and the resulting mixture was refluxed for 4 hrs at 50-60° C. After completion of the reaction, the reaction mixture was extracted using water and ethyl acetate and evaporated to dryness to yield compound 2 (yield: 68%). ¹H NMR (DMSO-d6) δ 1.26 (t, 3H), 4.24-4.26 (m, 2H), 4.67-4.69 (d, 2H), 6.64 (t, 1H), 6.77-6.79 (dd, 1H), 7.44-7.47 (m, 3H), 7.87-7.90 (dd, 2H), 8.03-8.05 (d, 1H), 8.69 (t, 1H).

Step-3: To a solution of compound 2 (6.41 g, 16.1 mmol) and NBu₄Br (1.02 g, 3.17 mmol, 0.19 eq) in dichloromethane (65 mL) was added 20% KOH (33.2 mL) and heated at 50° C. for 7 hrs. After completion of the reaction, the organic layer was separated, evaporated to dryness to yield compound 3 as an orange solid (yield: 67%).

Step-4: To a slurry of Rainey Nickel (0.2 g) in dioxane (10 mL) and THF (10 mL) was added to compound 3 (0.20 g, 0.66 mmol) and the resulting reaction mixture was hydrogenated under H₂ for 6 hrs. After completion of the reaction, the crude reaction mixture was filtered through Celite and solvent was evaporated. The residue was dissolved in dichloromethane and washed with water. Organic layer was dried over anhydrous Na₂SO₄ and evaporated to get a crude product. The crude product was purified by column chromatography using EtOAc in hexane (2:8) to yield compound 4 (yield: 89%).

Step-5: To a stirred solution of compound 4 (0.18, 0.66 mmol) in THF (5 mL) under argon was added CDI (0.11 g, 0.7 mmol, 1.1 eq) in portions and stirred at rt for 3-4 hrs. After completion of the reaction, the reaction mixture was evaporated to dryness to furnish a solid product that was washed with diethyl ether to give compound 5 in pure form (yield: 75%).

Step-6: To a solution of compound 5 (0.75 g, 2.53 mmol) in dioxane: methanol (10:8 mL) was added 1.18 M LiOH (8.6 mL) and stirred at rt for 12 hrs. After completion of the reaction, solvents were removed under reduced pressure and the reaction mixture was neutralized by acetic acid. The solid was collected by filtration to afford compound 6 (yield: 90%). ¹H NMR (DMSO-d6) δ 4.75-4.77 (d, 2H), 6.82-6.87 (m, 2H), 7.56 (m, 3H), 8.16-8.18 (d, 2H), 8.32-8.34 (m, 1H), 8.62 (bs, 1H).

Step-7: To a solution of compound 6 (0.37 g, 1.38 mmol) in DMF (5 mL) and NEt₃ (0.37 mL, 3 mmol, 2 eq) was added HBTU (0.57 g, 1.5 mmol, 1.1 eq) in DMF (2 mL) and stirred for 15 min at rt. O-(tetrahydro-2H-pyran-2-yl)-hydroxylamine (0.175 g, 1.5 mmol, 1.1 eq) in DMF (1 mL) was added to the solution. The resulting solution was stirred at rt for 12 hrs. After completion of the reaction, water was added to the reaction mass. The solid thus formed was filtered, dried and purified by washing with ether. The resulting solid of compound 7 was used as it is without any further purification (yield: 78%).

Step-8: To a solution of compound 7 (0.30 g, 0.817 mmol) in THF (4 mL) was added AcOH (8 mL) and water (2 mL). The resulting solution was stirred at 60° C. for 6 hrs. After completion of the reaction, the solvents were evaporated in vacuum. The solid thus formed was washed with water, filtered and recrystallized from ethanol to obtain the target compound HDAC-B-1 (Yield: 60%). ¹H NMR (DMSO-d6) δ 5.04 (s, 2H), 6.95-6.99 (m, 4H), 7.35 (dd, 2H), 7.69 (dd, 2H), 10.98 (bs, 1H); ¹³C NMR (400 MHz, DMSO) δ: 43.40, 61.83, 101.43, 108.48, 109.37, 121.04, 121.58, 127.70, 128.74, 130.28, 130.32, 132.40, 141.19, 154.78, 164.4; ESMS m/z 284.1 (M+1).

HDAC inhibitor compounds B2, B3, B4, B5, B6, and B7 were synthesized using the same synthetic scheme as given for HDAC inhibitor compound B1 with appropriate starting materials.

According to another embodiment, the present invention provides a process for preparing a substituted benzotriazole as an HDAC inhibitor. In some such embodiments, the inventive compounds are prepared as shown in the scheme below:

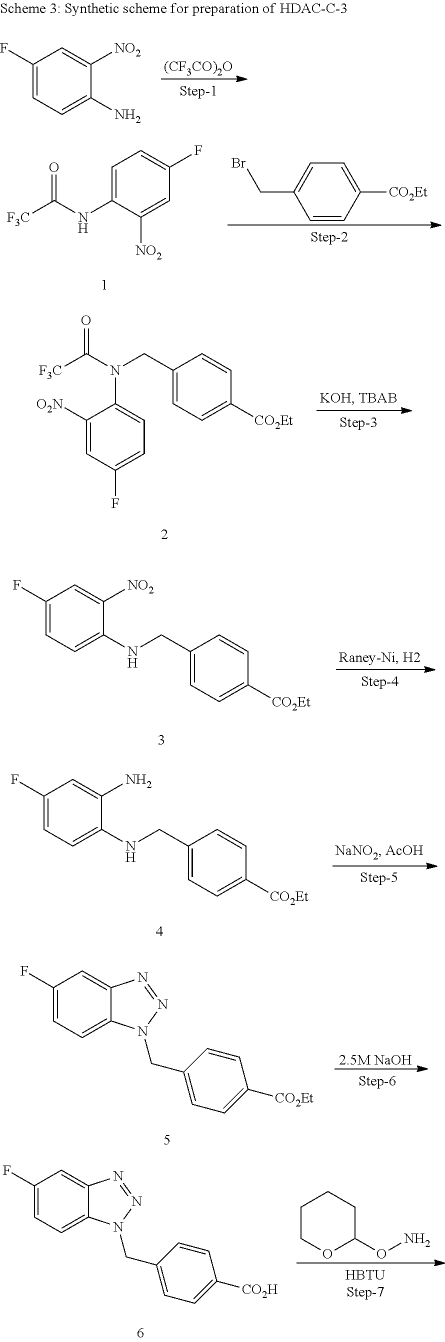

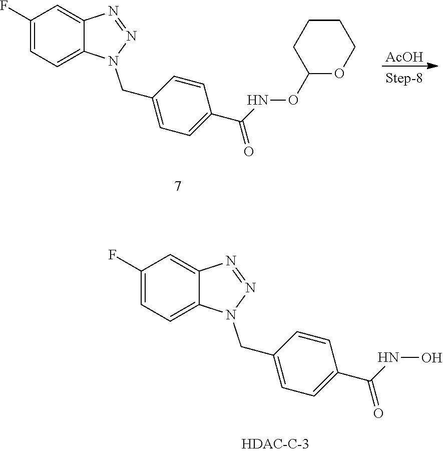 [SMILES: "AcOH" ; O=C(NOC1CCCCO1)c1ccc(Cn2nnc3cc(F)ccc32)cc1 ; O=C(NO)c1ccc(Cn2nnc3cc(F)ccc32)cc1]

Step-1: A solution of 4-fluoro-2-nitro aniline (10.0 g, 64 mmol, 1 eq) in dichloromethane (100 mL) was cooled to 0° C. and stirred for 30 minutes. Trifluoroacetic anhydride (17.8 mL, 128 mmol, 2 eq) was added and the reaction mixture was stirred at 0° C. for another 30 minutes. Completion of the reaction was monitored by TLC and NaHCO₃ was added to neutralize the reaction. The organic layer was separated and distilled to dryness to yield compound 1 as a yellow solid (Yield: 93%).

Step-2: To a solution of compound 1 (4.2 g, 16.7 mmol, 1 eq) in DMF (15 mL) was added Potassium carbonate (4.61 g, 33.4 mmol, 2 eq) and stirred at rt for 15 minutes. Ethyl 4-(bromomethyl)benzoate (4.21 g, 18.4 mmol, 1.1 eq) dissolved in DMF (5 mL) was added drop-wise and the resulting mixture was refluxed for 4 hrs at 50-60° C. Completion of the reaction was monitored by using TLC and the reaction mixture was extracted using water and ethyl acetate and evaporated to dryness to yield compound 2 (yield: 70%).

Step-3: To a solution of compound 2 (4.68 g, 11.5 mmol) and Tetrabutylammonium bromide (0.7 g, 2.2 mmol, 0.19 eq) in dichloromethane (40 mL) was added 20% KOH (24 mL) and heated at 50° C. for 7 hrs. Completion of the reaction was monitored by TLC and the organic layer was separated, evaporated to dryness to yield compound 3 (yield: 73%).

Step-4: To a slurry of Rainey Nickel (2 g) in dioxane (20 mL) and THF (40 mL) was added compound 3 (3.0 g, 9.8 mmol) and the resulting reaction mixture was hydrogenated under H₂ for 6 hrs. Completion of reaction was monitored by TLC and the crude reaction mixture was filtered through Celite and solvent was evaporated. The residue was dissolved in dichloromethane and washed with water. Organic layer was dried over anhydrous Na₂SO₄ and evaporated to get crude product which was purified by column chromatography using ethylacetate and hexane (2:8) to yield compound 4 (Yield: 74%).

Step-5: To a stirred solution of compound 4 (3.0 g, 10.4 mmol) in acetic acid (30 mL) and water at 0° C. was added drop-wise an aqueous solution of NaNO₂ (1.2 g, 17.4 mmol in 30 mL of water) and stirred at 0° C. for 2 hrs. Completion of the reaction was monitored by TLC, and the dark solid that formed was collected through filtration, which was washed with diethyl ether to give compound 5 (yield: 59%). ¹H NMR (DMSO-d6) δ 1.24-1.28 (t, 3H), 4.25-4.27 (m, 2H), 6.08 (2H), 7.41-7.42 (m, 3H), 7.90-7.92 (m, 4H).

Step-6: To a solution of compound 5 (0.40 g, 1.0 mmol) in methanol (10 mL) was added 2.5 M NaOH (3 mL) and stirred at rt for 12 hrs. The completion of the reaction was monitored by TLC. Solvents were removed under reduced pressure and the reaction mixture was neutralized by acetic acid. The solid was collected by filtration to afford compound 6 (yield: 88%).

Step-7: To a solution of compound 6 (0.200 g, 0.623 mmol) in DMF (5 mL) and triethylamine (0.15 mL, 1.25 mmol, 2 eq) was added HBTU (0.426 g, 0.747 mmol, 1.2 eq) in DMF (2 mL) and stirred for 15 minutes at rt. The O-(tetrahydro-2H-pyran-2-yl)-hydroxylamine (0.131 g, 0.747 mmol, 1.2 eq) in DMF (1 mL) was then added. The resulting solution was stirred at rt for 12 hrs. Completion of the reaction was monitored by TLC and water was added to the reaction mixture. The solid thus formed was filtered, dried and purified by washing with ether. The solid obtained was used as it is for the next step without any further purification (yield: 84%). ¹H NMR (DMSO-d6) δ 1.49 (m, 3H), 1.68 (m, 3H), 2.47 (s, 1H), 4.03 (s, 1H), 4.99 (s, 1H), 6.02 (s, 2H), 7.40 (dd, 2H), 7.46 (m, 1H), 7.68 (d, 2H), 7.90 (d, 2H), 11.60 (s, 1H).

Step-8: To a solution of compound 7 (0.490 g, 1.16 mmol) in THF (4 mL) was added acetic acid (8 mL) and water (2 mL). The resulting solution was stirred at 60° C. for 6 hrs. Completion of the reaction was monitored by TLC and the solvents were evaporated in vacuo. The solid thus formed was washed with water, filtered, dried and purified by preparative TLC using 50% ethylacetate in Hexane to obtain the target compound HDAC-C-3 (yield: 63%). ¹H NMR (DMSO-d6) δ 5.91 (s, 2H), 7.24-7.36 (d, 3H), 7.57-7.59 (m, 2H), 7.76-7.78 (m, 2H); ¹³C NMR (400 MHz, DMSO) δ: 51.23, 104.14, 104.38, 112.75, 117.70, 117.97, 127.85, 130.40, 133.07, 139.03, 145.82, 158.43, 160.82, 164.20; ESMS m/z 287.0 (M+1).

HDAC inhibitor compounds C1, C2, C4, C5, were synthesized using the same synthetic scheme as given for the HDAC inhibitor compound C3 using the appropriate starting materials.

### Compositions Comprising HDAC Inhibitors

According to another aspect, the present invention further provides compositions comprising an effective amount of at least one of HDAC inhibitor of Formula I:

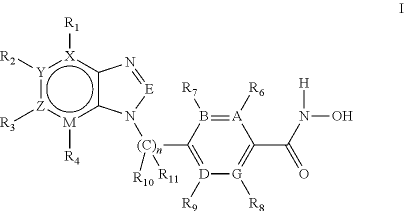

or a pharmaceutically acceptable salt thereof, and a carrier, wherein:

each of X, Y, Z and M is independently C or N;

each of R₁, R₂, R₃ and R₄ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₁, R₂, R₃ and R₄ is H or a substituent when X, Y, Z and M is carbon;

E is C—R₅, or N;

R₅ is H, OH, NH₂, amino optionally substituted by alkyl or aryl, CN, F, Cl, Br, I, C₁-C₆ perfluoroalkyl, O-alkyl, O-aryl, O-heteroaryl, NO₂, cycloalkyl, aryl, acyl, optionally substituted C₁-C₆ alkyl, C₂-C₆ alkene, or C₂-C₆ alkyne, wherein when R₅ is OH, the compound exists as a keto tautomer, as an enol tautomer or as a mixture of keto-enol tautomers;

each of A, B, D, and G is independently C or N;

each of R₆, R₇, R₈, and R₉ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₆, R₇, R₈ and R₉ is H or a substituent when A, B, D and G is carbon;

each of R₁₀ and R₁₁ is independently H, alkyl, or aryl, wherein (C)ₙ optionally is a chiral center, wherein (C)ₙ can exist as both R and S enantiomers, with the proviso that when R₁₀ is H, R₁₁ is alkyl or aryl; and when R₁₁ is H, R₁₀ is alkyl or aryl; and

n is 0, 1, 2, 3, 4, 5, 6, 7, 8, 9 or 10.

According to some embodiments, the present invention further provides a composition of an effective amount of at least one compound of Formula Ia:

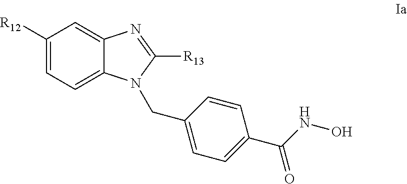

or a pharmaceutically acceptable salt thereof, and a carrier, wherein:

R₁₂ is H, alkyl, F, Cl, Br, I, or O-alkyl; and

R₁₃ is H or C₁-C₆ perfluoroalkyl.

According to some embodiments, the present invention further provides a composition comprising an effective amount of at least one compound of Formula Ib:

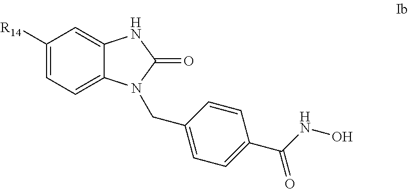

or a pharmaceutically acceptable salt thereof, and a carrier, wherein:

R₁₄ is H, alkyl, F, Cl, Br, I, O-alkyl, or C₁-C₆ perfluoroalkyl.

According to some embodiments, the present invention further provides a composition comprising an effective amount of at least one compound of Formula Ic:

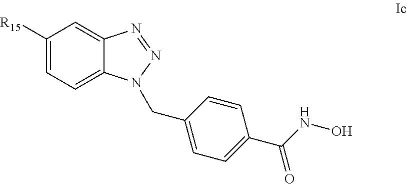

or a pharmaceutically acceptable salt thereof, and a carrier, wherein:

R₁₅ is H, alkyl, F, Cl, Br, I, or O-alkyl.

For any compound described herein the therapeutically effective amount may be initially determined from preliminary in vitro studies and/or animal models. A therapeutically effective dose may also be determined from human data for HDAC inhibitors. The applied dose may be adjusted based on the relative bioavailability and potency of the administered compound. Adjusting the dose to achieve maximal efficacy based on the methods described above and other methods as are well-known in the art is well within the capabilities of the ordinarily skilled artisan.

According to some embodiments, provided compounds are considered HDAC inhibitors in that they inhibit the histone deacetylating activity of histone deacetylase enzymes, i.e., the removal of acetyl groups from an acetylated ε-amino group of a conserved lysine residue on a histone. According to some embodiments, provided compounds are inhibitors of HDAC1, HDAC2, HDAC3, HDAC4, HDAC5, HDAC6, HDAC7, HDAC8, HDAC9, HDAC10, HDAC11, or a combination thereof. According to some embodiments, provided compounds are inhibitors of HDAC1. According to some embodiments, provided compounds are inhibitors of HDAC2. According to some embodiments, provided compounds are inhibitors of HDAC3. According to some embodiments, provided compounds are inhibitors of HDAC4. According to some embodiments, provided compounds are inhibitors of HDAC5. According to some embodiments, provided compounds are inhibitors of HDAC6. According to some embodiments, provided compounds are inhibitors of HDAC7. According to some embodiments, provided compounds are inhibitors of HDAC8. According to some embodiments, provided compounds are inhibitors of HDAC9. According to some embodiments, provided compounds are inhibitors of HDAC10. According to some embodiments, provided compounds are inhibitors of HDAC11. According to some embodiments, provided compounds are selective inhibitors of HDAC6.

According to some embodiments, the HDAC inhibitor inhibits the histone deacetylating activity of an HDAC isoform selected from the group consisting of HDAC1, HDAC2, HDAC3, HDAC4, HDAC5, HDAC7, HDAC8, HDAC9, HDAC10, HDAC11 with a histone deacetylase inhibition activity (IC₅₀) in vitro ranging from about 0.005 μM to about 3 μM. According to some embodiments, the HDAC inhibitor inhibits the histone deacetylating activity of an HDAC isoform selected from the group consisting of HDAC1, HDAC2, HDAC3, HDAC4, HDAC5, HDAC7, HDAC8, HDAC9, HDAC10, HDAC11 with a histone deacetylase inhibition activity (IC₅₀) in vitro of at least about 0.005 μM, at least about 0.010 μM, at least about 0.020 μM, at least about 0.030 μM, at least about 0.040 μM, at least about 0.050 μM, at least about 0.060 μM, at least about 0.070 μM, at least about 0.080 μM, at least about 0.090 μM, at least about 0.1 μM, at least about 0.2 μM, at least about 0.3 μM, at least about 0.4 μM, at least about 0.5 μM, at least about 0.6 μM, at least about 0.7 μM, at least about 0.8 μM, at least about 0.9 μM, at least about 1 μM, at least about 1.1 μM, at least about 1.2 μM, at least about 1.3 μM, at least about 1.4 μM, at least about 1.5 μM, at least about 1.6 μM, at least about 1.7 μM, at least about 1.8 μM, at least about 1.9 μM, at least about 2 μM, at least about 2.1 μM, at least about 2.2 μM, at least about 2.3 μM, at least about 2.4 μM, at least about 2.5 μM, at least about 2.6 μM, at least about 2.7 μM, at least about 2.8 μM, at least about 2.9 μM.

According to some embodiments, the HDAC inhibitor selectively inhibits the histone deacetylating activity of HDAC6. According to one embodiment, the HDAC inhibitor inhibits the histone deacetylating activity of HDAC6 with an inhibition activity (IC₅₀) in vitro ranging from about 0.000001 μM to about 0.001 μM. According to one embodiment, the histone deacetylase inhibition activity (IC₅₀) in vitro is at least about 0.000001 μM. According to another embodiment, the histone deacetylase inhibition activity (IC₅₀) in vitro is at least about 0.000005 μM. According to another embodiment, the histone deacetylase inhibition activity (IC₅₀) in vitro is at least about 0.00001 μM. According to another embodiment, the histone deacetylase inhibition activity (IC₅₀) in vitro is at least about 0.00005 μM. According to another embodiment, the histone deacetylase inhibition activity (IC₅₀) is at least about 0.0001 μM. According to another embodiment, the histone deacetylase inhibition activity (IC₅₀) in vitro is at least about 0.0005 μM. According to another embodiment, the histone deacetylase inhibition activity (IC₅₀) in vitro is about 0.001 μM.

According to some embodiments, the HDAC inhibitor is selective toward HDAC6. According to one embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 100. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 500. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 1,000. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 5,000. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 10,000. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 20,000. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 30,000.

According to one embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 2.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 4.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 6.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 8.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 10.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 15.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 20.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 25.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 30.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 35.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 40.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 45.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 55.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 60.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 65.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 70.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 75.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 80.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 85.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 90.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 95.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 100.0.

The formulations of inhibitors may be administered in pharmaceutically acceptable solutions, which may routinely contain pharmaceutically acceptable concentrations of salt, buffering agents, preservatives, compatible carriers, adjuvants, and optionally other therapeutic agents.

According to another embodiment, the compositions of the present invention can further include one or more additional compatible active ingredients. “Compatible” as used herein means that the components of such a composition are capable of being combined with each other in a manner such that there is no interaction that would substantially reduce the efficacy of the composition under ordinary use conditions.

In one embodiment, the compound of the inventive compositions is an active ingredient.

Additional active ingredients included in the compositions according to the present invention used to inhibit HDAC include, without limitation, one or more, in any combination, of an antibiotic agent, an antifungal agent, an antiviral agent, an antiprotozoal agent, an anesthetic agent, an anti-inflammatory agent, an antipruritic agent, an anti-oxidant agent, a chemotherapeutic agent, an anti-histamine agent, a vitamin, or a hormone.

Examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefmetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

Anti-fungal agents include, but are not limited to, Amphotericin B, Candicidin, Dermostatin, Filipin, Fungichromin, Hachimycin, Hamycin, Lucensomycin, Mepartricin, Natamycin, Nystatin, Pecilocin, Perimycin, Azaserine, Griseofulvin, Oligomycins, Neomycin, Pyrrolnitrin, Siccanin, Tubercidin, Viridin, Butenafine, Naftifine, Terbinafine, Bifonazole, Butoconazole, Chlordantoin, Chlormidazole, Cloconazole, Clotrimazole, Econazole, Enilconazole, Fenticonazole, Flutrimazole, Isoconazole, Ketoconazole, Lanoconazole, Miconazole, Omoconazole, Oxiconazole, Sertaconazole, Sulconazole, Tioconazole, Tolciclate, Tolindate, Tolnaftate, Fluconazole, Itraconazole, Saperconazole, Terconazole, Acrisorcin, Amorolfine, Biphenamine, Bromosalicylchloranilide, Buclosamide, Calcium Propionate, Chlorphenesin, Ciclopirox, Cloxyquin, Coparaffinate, Diamthazole, Exalamide, Flucytosine, Halethazole, Hexetidine, Loflucarban, Nifuratel, Potassium Iodide, Propionic Acid, Pyrithione, Salicylanilide, Sodium Propionate, Sulbentine, Tenonitrozole, Triacetin, Ujothion, Undecylenic Acid, and Zinc Propionate.

Anti-viral agents include, but are not limited to, Acyclovir, Cidofovir, Cytarabine, Dideoxyadenosine, Didanosine, Edoxudine, Famciclovir, Floxuridine, Ganciclovir, Idoxuridine, Inosine Pranobex, Lamivudine, MADU, Penciclovir, Sorivudine, Stavudine, Trifluridine, Valacyclovir, Vidarabine, Zalcitabine, Zidovudine, Acemannan, Acetylleucine, Amantadine, Amidinomycin, Delavirdine, Foscamet, Indinavir, Interferons (e.g., IFN-alpha), Kethoxal, Lysozyme, Methisazone, Moroxydine, Nevirapine, Podophyllotoxin, Ribavirin, Rimantadine, Ritonavir2, Saquinavir, Stailimycin, Statolon, Tromantadine, Zidovudine (AZT) and Xenazoic Acid.

Examples of antiprotozoal agents, without limitation include pyrimethamine (Daraprim®) sulfadiazine, and Leucovorin.

Non-limiting examples of anesthetic drugs that are suitable for use in the context of the present invention include pharmaceutically acceptable salts of lidocaine, bupivacaine, chlorprocaine, dibucaine, etidocaine, mepivacaine, tetracaine, dyclonine, hexylcaine, procaine, cocaine, ketamine, pramoxine and phenol.

Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

Examples of non-steroidal anti-inflammatory agents that are usable in the context of the present invention include, without limitation, ibuprofen (Advil)®, naproxen sodium (Aleve)®, and acetaminophen (Tylenol)®, and oxicams, such as piroxicam, isoxicam, tenoxicam, sudoxicam, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed, as well as the dermatologically acceptable salts and esters of these agents. For example, etofenamate, a flufenamic acid derivative, is particularly useful for topical application.

Suitable antipruritic agents include, without limitation, pharmaceutically acceptable salts of methdilazine and trimeprazine.

Non-limiting examples of anti-oxidants that are usable in the context of the present invention include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename TroloxR), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, glycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

Non-limiting examples of chemotherapeutic agents usable in context of the present invention include daunorubicin, doxorubicin, idarubicin, amrubicin, pirarubicin, epirubicin, mitoxantrone, etoposide, teniposide, vinblastine, vincristine, mitomycin C, 5-FU, paclitaxel, docetaxel, actinomycin D, colchicine, topotecan, irinotecan, gemcitabine cyclosporin, verapamil, valspodor, probenecid, MK571, GF120918, LY335979, biricodar, terfenadine, quinidine, pervilleine A and XR9576.

Non-limiting examples of antihistamines usable in context of the present invention include chlorpheniramine, brompheniramine, dexchlorpheniramine, tripolidine, clemastine, diphenhydramine, promethazine, piperazines, piperidines, astemizole, loratadine and terfenadine.

Non-limiting examples of vitamins usable in context of the present invention include vitamin A and its analogs and derivatives: retinol, retinal, retinyl palmitate, retinoic acid, tretinoin, iso-tretinoin (known collectively as retinoids), vitamin E (tocopherol and its derivatives), vitamin C (L-ascorbic acid and its esters and other derivatives), vitamin B3 (niacinamide and its derivatives), alpha hydroxy acids (such as glycolic acid, lactic acid, tartaric acid, malic acid, citric acid, etc.) and beta hydroxy acids (such as salicylic acid and the like).

Examples of hormones for use in the context of the present invention include, but are not limited to, calciferol (Vitamin D3) and its products, androgens, estrogens and progesterones.

A subject in need thereof is a patient having, or at risk of having a disorder in which HDAC plays a direct or indirect role. According to some embodiments, the term “subject in need of such treatment” also is used to refer to a patient who (i) will be administered at least one HDAC inhibitor of the invention, (ii) is receiving at least one HDAC inhibitor of the invention, or (iii) has received at least one HDAC inhibitor of the invention, unless the context and usage of the phrase indicates otherwise.

### Administration

For use in therapy, a therapeutically effective amount of the HDAC inhibitor may be administered to a subject by any mode, and administering the pharmaceutical composition may be accomplished by any means known to the skilled artisan. Routes of administration include, but are not limited to, intrathecal, intra-arterial, parenteral, intramuscular, oral, buccal, topical, by inhalation or insufflation (i.e, through the mouth or through the nose), or rectal.

### Parenteral Administration

The HDAC inhibitor, when it is desirable to deliver it locally, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension also may contain suitable stabilizers or agents, which increase the solubility of the compounds to allow for the preparation of highly concentrated solutions. Alternatively, the active compounds may be in powder form for constitution with a suitable vehicle, e.g., sterile pyrogen-free water, before use.

The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Suitable liquid or solid pharmaceutical preparation forms are, for example, microencapsulated, and if appropriate, with one or more excipients, encochleated, coated onto microscopic gold particles, contained in liposomes, pellets for implantation into the tissue, or dried onto an object to be rubbed into the tissue. Such pharmaceutical compositions also may be in the form of granules, beads, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, or solubilizers are customarily used as described above. The pharmaceutical compositions are suitable for use in a variety of drug delivery systems. For a brief review of methods for drug delivery, see Langer 1990 Science 249, 1527-1533, which is incorporated herein by reference.

### Pharmaceutically Acceptable Salts

Depending upon the structure, at least one inhibitor of the described invention, and optionally at least one other therapeutic agent, may be administered per se (neat) or, depending upon the structure of the inhibitor, in the form of a pharmaceutically acceptable salt. The inhibitors of the described invention may form pharmaceutically acceptable salts with organic or inorganic acids, or organic or inorganic bases. When used in medicine the salts should be pharmaceutically acceptable, but non-pharmaceutically acceptable salts conveniently may be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts may be prepared as alkaline metal or alkaline earth salts, such as sodium, potassium or calcium salts of the carboxylic acid group.

By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002).

The salts may be prepared in situ during the final isolation and purification of the compounds described within the present invention or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate(isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides, such as methyl, ethyl, propyl, and butyl chlorides, bromides and iodides; dialkyl sulfates like dimethyl, diethyl, dibutyl and diamyl sulfates; long chain halides, such as decyl, lauryl, myristyl and stearyl chlorides, bromides and iodides; arylalkyl halides, such as benzyl and phenethyl bromides, and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid. Basic addition salts may be prepared in situ during the final isolation and purification of compounds described within the invention by reacting a carboxylic acid-containing moiety with a suitable base such as the hydroxide, carbonate or bicarbonate of a pharmaceutically acceptable metal cation or with ammonia or an organic primary, secondary or tertiary amine Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts may be also obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids may also be made.

The formulations may be presented conveniently in unit dosage form and may be prepared by any of the methods well known in the art of pharmacy. All methods include the step of bringing into association an HDAC inhibitor, or a pharmaceutically acceptable salt or solvate thereof (“active compound”) with the carrier which constitutes one or more accessory agents. In general, the formulations are prepared by uniformly and intimately bringing into association the active agent with liquid carriers or finely divided solid carriers or both and then, if necessary, shaping the product into the desired formulation.

The pharmaceutical agent or a pharmaceutically acceptable ester, salt, solvate or prodrug thereof may be mixed with other active materials that do not impair the desired action, or with materials that supplement the desired action. Solutions or suspensions used for parenteral, intradermal, subcutaneous, intrathecal, or topical application may include, but are not limited to, for example, the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation may be enclosed in ampoules, disposable syringes or multiple dose vials made of glass or plastic. Administered intravenously, particular carriers are physiological saline or phosphate buffered saline (PBS).

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions also may contain adjuvants including preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

Injectable depot forms are made by forming microencapsulated matrices of a described inhibitor in biodegradable polymers such as polylactide-polyglycolide. Depending upon the ratio of inhibitor to polymer and the nature of the particular polymer employed, the rate of drug release may be controlled. Such long acting formulations may be formulated with suitable polymeric or hydrophobic materials (for example as an emulsion in an acceptable oil) or ion exchange resins, or as sparingly soluble derivatives, for example, as a sparingly soluble salt. Examples of other biodegradable polymers include poly(orthoesters) and poly(anhydrides). Depot injectable formulations also are prepared by entrapping the inhibitor of the described invention in liposomes or microemulsions, which are compatible with body tissues.

The locally injectable formulations may be sterilized, for example, by filtration through a bacterial-retaining filter or by incorporating sterilizing agents in the form of sterile solid compositions that may be dissolved or dispersed in sterile water or other sterile injectable medium just prior to use. Injectable preparations, for example, sterile injectable aqueous or oleaginous suspensions may be formulated according to the known art using suitable dispersing or wetting agents and suspending agents. The sterile injectable preparation also may be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils conventionally are employed or as a solvent or suspending medium. For this purpose any bland fixed oil may be employed including synthetic mono- or diglycerides. In addition, fatty acids such as oleic acid are used in the preparation of injectables.

Formulations for parenteral administration include aqueous and non-aqueous sterile injection solutions that may contain anti-oxidants, buffers, bacteriostats and solutes, which render the formulation isotonic with the blood of the intended recipient; and aqueous and non-aqueous sterile suspensions, which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example, saline, water-for-injection, immediately prior to use. Extemporaneous injection solutions and suspensions may be prepared from sterile powders, granules and tablets of the kind previously described.

Examples of suitable buffering agents include, without limitation: acetic acid and a salt (1%-2% w/v); citric acid and a salt (1%-3% w/v); boric acid and a salt (0.5%-2.5% w/v); and phosphoric acid and a salt (0.8%-2% w/v). Suitable preservatives include benzalkonium chloride (0.003%-0.03% w/v); chlorobutanol (0.3%-0.9% w/v); parabens (0.01%-0.25% w/v) and thimerosal (0.004%-0.02% w/v).

### Oral Administration

For oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents also may be incorporated in the mixture. Powders and tablets may be comprised of from about 5 to about 95 percent inventive composition. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum and the like.

Sweetening and flavoring agents and preservatives may also be included where appropriate.

Liquid form preparations include solutions, suspensions and emulsions. As an example may be mentioned water or water-propylene glycol solutions for parenteral injections or addition of sweeteners and pacifiers for oral solutions, suspensions and emulsions. Liquid form preparations also may include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides, such as cocoa butter, is first melted, and the active ingredient is dispersed homogeneously therein by stirring or similar mixing. The molten homogeneous mixture is then poured into convenient sized molds, allowed to cool and thereby solidify.

Also included are solid form preparations, which are intended to be converted, shortly before use, to liquid form preparations for either oral or parenteral administration. Such liquid forms include solutions, suspensions and emulsions.

The compounds of the described invention also may be deliverable transdermally. The transdermal compositions may take the form of creams, lotions, aerosols and/or emulsions and can be included in a transdermal patch of the matrix or reservoir type as are conventional in the art for this purpose.

Conventional methods for preparing tablets are known. Such methods include dry methods such as direct compression and compression of granulation produced by compaction, or wet methods or other special procedures. Conventional methods for making other forms for administration such as, for example, capsules, suppositories and the like are also well known.

### Pharmaceutically Acceptable Carrier

The pharmaceutical compositions within the described invention contain a therapeutically effective amount of an HDAC inhibitor and optionally other therapeutic agents included in a pharmaceutically-acceptable carrier. The components of the pharmaceutical compositions also are capable of being commingled in a manner such that there is no interaction which would substantially impair the desired pharmaceutical efficiency.

The therapeutic agent(s), including the HDAC inhibitor(s) of the described invention may be provided in particles. The particles may contain the therapeutic agent(s) in a core surrounded by a coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, etc., and any combination thereof. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules that contain the HDAC inhibitor in a solution or in a semi-solid state. The particles may be of virtually any shape.

Both non-biodegradable and biodegradable polymeric materials may be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels as described by Sawhney et al in Macromolecules (1993) 26, 581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. In order to prolong the effect of a drug, it often is desirable to slow the absorption of the drug from subcutaneous, intrathecal, or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Use of a long-term sustained release formulations may be particularly suitable for treatment of chronic conditions. Long-term sustained release formulations are well-known to those of ordinary skill in the art and include some of the release systems described above.

### Kits Comprising HDAC Inhibitors

According to another aspect, the described invention provides kits for treating diseases associated with HDACs.

A kit for treating a histone deacetylase (HDAC)-associated disease, comprising a pharmaceutical composition comprising (a) a therapeutic amount of at least one HDAC inhibitor of Formula I:

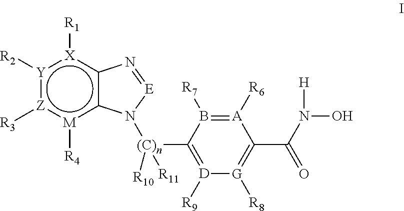

or a pharmaceutically acceptable salt thereof, and a carrier, wherein:

each of X, Y, Z and M is independently C or N;

each of R₁, R₂, R₃ and R₄ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₁, R₂, R₃ and R₄ is H or a substituent when X, Y, Z and M is carbon;

E is C—R₅, or N;

R₅ is H, OH, NH₂, amino optionally substituted by alkyl or aryl, CN, F, Cl, Br, I, C₁-C₆ perfluoroalkyl, O-alkyl, O-aryl, O-heteroaryl, NO₂, cycloalkyl, aryl, acyl, optionally substituted C₁-C₆ alkyl, C₂-C₆ alkene, or C₂-C₆ alkyne, wherein when R₅ is OH, the compound exists as a keto tautomer, as an enol tautomer or as a mixture of keto-enol tautomers;

each of A, B, D, and G is independently C or N;

each of R₆, R₇, R₈, and R₉ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₆, R₇, R₈ and R₉ is H or a substituent when A, B, D and G is carbon;

each of R₁₀ and R₁₁ is independently H, alkyl, or aryl, wherein (C)ₙ optionally is a chiral center, wherein (C)ₙ can exist as both R and S enantiomers, with the proviso that when R₁₀ is H, R₁₁ is alkyl or aryl; and when R₁₁ is H, R₁₀ is alkyl or aryl; and
n is 0, 1, 2, 3, 4, 5, 6, 7, 8, 9 or 10;

(b) a pharmaceutically acceptable carrier, wherein the therapeutic amount is effective to inhibit the activity of at least one HDAC isoform and in treating symptoms of the HDAC-associated disease, and

(c) a means for administering the composition.

According to some embodiments, a kit for treating an HDAC-associated disease, disorder or condition comprises a form containing a composition comprising a therapeutically effective amount of at least one HDAC inhibitor of Formula Ia:

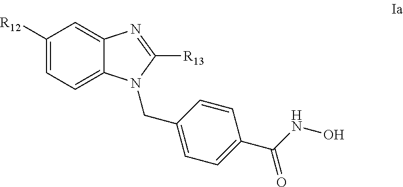

or a pharmaceutically acceptable salt thereof, and a carrier, wherein:

R₁₂ is H, alkyl, F, Cl, Br, I, or O-alkyl; and

R₁₃ is H or C₁-C₆ perfluoroalkyl.

According to some embodiments, a kit for treating an HDAC-associated disease, disorder or condition comprises a form containing a composition comprising a therapeutically effective amount of at least one HDAC inhibitor of Formula Ib:

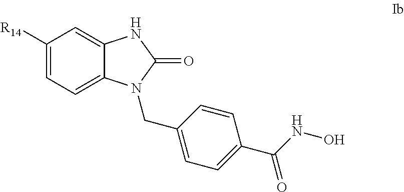

or a pharmaceutically acceptable salt thereof, and a carrier, wherein:

R₁₄ is H, alkyl, F, Cl, Br, I, O-alkyl, or C₁-C₆ perfluoroalkyl.

According to some embodiments, a kit for treating an HDAC-associated disease comprises a form containing a composition comprising an effective amount of at least one HDAC inhibitor of Formula Ic:

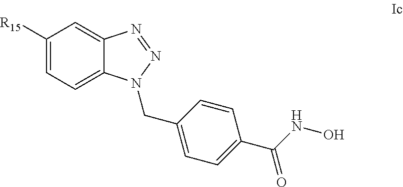

or a pharmaceutically acceptable salt thereof, and a carrier, wherein:

R₁₅ is H, alkyl, F, Cl, Br, I, or O-alkyl.

According to some embodiments, the HDAC inhibitor inhibits histone deacetylating activity of at least one HDAC isoform selected from the group consisting of HDAC1, HDAC2, HDAC3, HDAC4, HDAC5, HDAC6, HDAC7, HDAC8, HDAC9, and a combination thereof.

According to some embodiments, the HDAC inhibitor is selective toward HDAC6. According to one embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 100. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 500. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 1,000. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 5,000. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 10,000. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 20,000. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 30,000.

According to one embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 2.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 4.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 6.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 8.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 10.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 15.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 20.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 25.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 30.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 35.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 40.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 45.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 55.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 60.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 65.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 70.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 75.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 80.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 85.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 90.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 95.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 100.0.

According to some embodiments, the means for administering the composition is a syringe, a nebulizer, an inhaler, or a combination thereof.

According to some embodiments, the kit further comprises instructions. According to some embodiments, the kit further comprises packaging materials. According to some embodiments, the form may be selected from a tablet, a capsule, a pill, a lozenge, a gel, an injectable solution, a powder, and an aerosol, etc. According to some embodiments, the packaging material may be selected from a box, a pouch, a vial, a bottle, a tube, etc.

### Methods for Inhibiting HDACs

According to another aspect, the present disclosure provides a method for inhibiting an HDAC in a subject in need thereof, the method comprising administering a pharmaceutical composition comprising a therapeutically effective amount of a compound of Formula I:

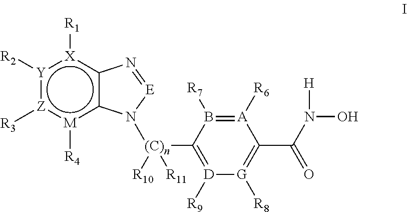

or a pharmaceutically acceptable salt thereof, and a carrier, wherein:

each of X, Y, Z and M is independently C or N;

each of R₁, R₂, R₃ and R₄ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₁, R₂, R₃ and R₄ is H or a substituent when X, Y, Z and M is carbon;

E is C—R₅, or N;

R₅ is H, OH, NH₂, amino optionally substituted by alkyl or aryl, CN, F, Cl, Br, I, C₁-C₆ perfluoroalkyl, O-alkyl, O-aryl, O-heteroaryl, NO₂, cycloalkyl, aryl, acyl, optionally substituted C₁-C₆ alkyl, C₂-C₆ alkene, or C₂-C₆ alkyne, wherein when R₅ is OH, the compound exists as a keto tautomer, as an enol tautomer or as a mixture of keto-enol tautomers;

each of A, B, D, and G is independently C or N;

each of R₆, R₇, R₈, and R₉ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₆, R₇, R₈ and R₉ is H or a substituent when A, B, D and G is carbon;

each of R₁₀ and R₁₁ is independently H, alkyl, or aryl, wherein (C)ₙ optionally is a chiral center, wherein (C)ₙ can exist as both R and S enantiomers, with the proviso that when R₁₀ is H, R₁₁ is alkyl or aryl; and when R₁₁ is H, R₁₀ is alkyl or aryl; and

n is 0, 1, 2, 3, 4, 5, 6, 7, 8, 9 or 10.

According to some embodiments, the present invention provides a method for inhibiting an HDAC in a subject, the method comprising administering a pharmaceutical composition comprising a therapeutically effective amount of a compound of Formula Ia:

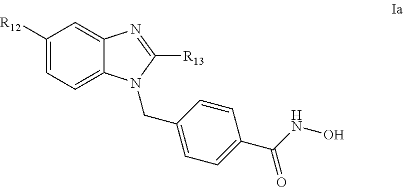

or a pharmaceutically acceptable salt thereof, and a carrier, wherein:

R₁₂ is H, alkyl, F, Cl, Br, I, or O-alkyl; and

R₁₃ is H or C₁-C₆ perfluoroalkyl.

According to some embodiments, the present invention provides a method for inhibiting an HDAC in a subject, the method comprising administering a pharmaceutical composition comprising a therapeutically effective amount of a compound of Formula Ib:

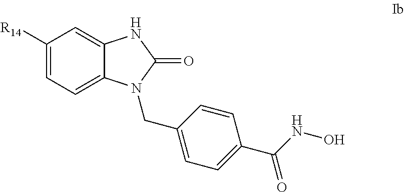

or a pharmaceutically acceptable salt thereof, and a carrier, wherein:

R₁₄ is H, alkyl, F, Cl, Br, I, O-alkyl, or C₁-C₆ perfluoroalkyl.

According to some embodiments, the present invention provides a method for inhibiting an HDAC in a subject, the method comprising administering a pharmaceutical composition comprising a therapeutically effective amount of a compound of Formula Ic:

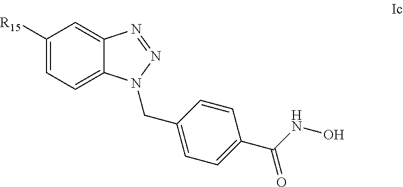

or a pharmaceutically acceptable salt thereof, and a carrier, wherein:

R₁₅ is H, alkyl, F, Cl, Br, I, or O-alkyl.

According to another embodiment, the HDAC is selected from the group consisting of HDAC1, HDAC2, HDAC3, HDAC4, HDAC5, HDAC6, HDAC7, HDAC8, HDAC7, HDAC8, HDAC9, HDAC10, HDAC11, and a combination thereof. According to another embodiment, the HDAC is HDAC1. According to another embodiment, the HDAC is HDAC2. According to another embodiment, the HDAC is HDAC3. According to another embodiment, the HDAC is HDAC4. According to another embodiment, the HDAC is HDAC5. According to another embodiment, the HDAC is HDAC6. According to another embodiment, the HDAC is HDAC7. According to another embodiment, the HDAC is HDAC8. According to another embodiment, the HDAC is HDAC9. According to another embodiment, the HDAC is HDAC10. According to another embodiment, the HDAC is HDAC11.

According to another embodiment, the therapeutically effective amount of the HDAC inhibitor is from about 1 pg/day to about 15 g/day.

According to another embodiment, the therapeutically effective amount of the HDAC inhibitor is from about 0.000001 mg/kg body weight to about 10 g/kg body weight. According to another embodiment, the therapeutically effective amount of the HDAC inhibitor is from about 0.000002 mg/kg body weight to about 10 g/kg body weight. According to another embodiment, the therapeutically effective amount of the HDAC inhibitor is from about 0.000003 mg/kg body weight to about 10 g/kg body weight. According to another embodiment, the therapeutically effective amount of the HDAC inhibitor is from about 0.000004 mg/kg body weight to about 10 g/kg body weight. According to another embodiment, the therapeutically effective amount of the HDAC inhibitor is from about 0.000005 mg/kg body weight to about 10 g/kg body weight. According to another embodiment, the therapeutically effective amount of the HDAC inhibitor is from about 0.000006 mg/kg body weight to about 10 g/kg body weight. According to another embodiment, the therapeutically effective amount of the HDAC inhibitor is from about 0.000007 mg/kg body weight to about 10 g/kg body weight. According to another embodiment, the therapeutically effective amount of the HDAC inhibitor is from about 0.000008 mg/kg body weight to about 10 g/kg body weight. According to another embodiment, the therapeutically effective amount of the HDAC inhibitor is from about 0.000009 mg/kg body weight to about 10 g/kg body weight. According to another embodiment, the therapeutically effective amount of the HDAC inhibitor is from about 0.00001 mg/kg body weight to about 10 g/kg body weight. According to another embodiment, the therapeutically effective amount of the HDAC inhibitor is from about 0.00002 mg/kg body weight to about 10 g/kg body weight. According to another embodiment, the therapeutically effective amount of the HDAC inhibitor is from about 0.0003 mg/kg body weight to about 10 g/kg body weight. According to another embodiment, the therapeutically effective amount of the HDAC inhibitor is from about 0.00004 mg/kg body weight to about 10 g/kg body weight. According to another embodiment, the therapeutically effective amount of the HDAC inhibitor is from about 0.00005 mg/kg body weight to about 10 g/kg body weight. According to another embodiment, the therapeutically effective amount of the HDAC inhibitor is from about 0.00006 mg/kg body weight to about 10 g/kg body weight. According to another embodiment, the therapeutically effective amount of the HDAC inhibitor is from about 0.00007 mg/kg body weight to about 10 g/kg body weight. According to another embodiment, the therapeutically effective amount of the HDAC inhibitor is from about 0.00008 mg/kg body weight to about 10 g/kg body weight. According to another embodiment, the therapeutically effective amount of the HDAC inhibitor is from about 0.00009 mg/kg body weight to about 10 g/kg body weight. According to another embodiment, the therapeutically effective amount of the HDAC inhibitor is from about 0.0001 mg/kg body weight to about 10 g/kg body weight. According to some such embodiments, the therapeutically effective amount of the HDAC inhibitor is about 0.0005 mg/kg body weight. According to some such embodiments, the therapeutically effective amount of the HDAC inhibitor is about 0.001 mg/kg body weight. According to some such embodiments, the therapeutically effective amount of the HDAC inhibitor is about 0.005 mg/kg body weight. According to some such embodiments, the therapeutically effective amount of the HDAC inhibitor is about 0.01 mg/kg body weight. According to some such embodiment, the therapeutically effective amount is about 0.1 mg/kg body weight. According to some such embodiments, the therapeutically effective amount of the HDAC inhibitor is about 1 mg/kg body weight. According to some such embodiments, the therapeutically effective amount of the HDAC inhibitor is about 10 mg/kg body weight. According to some such embodiments, the therapeutically effective amount of the HDAC inhibitor is about 20 mg/kg body weight. According to some such embodiments, the therapeutically effective amount of the HDAC inhibitor is about 30 mg/kg body weight. According to some such embodiments, the therapeutically effective amount of the HDAC inhibitor is about 40 mg/kg body weight. According to some such embodiments, the therapeutically effective amount of the HDAC inhibitor is about 50 mg/kg body weight. According to some such embodiments, the therapeutically effective amount of the HDAC inhibitor is about 60 mg/kg body weight. According to some such embodiments, the therapeutically effective amount of the HDAC inhibitor is about 70 mg/kg body weight. According to some such embodiments, the therapeutically effective amount of the HDAC inhibitor is about 80 mg/kg body weight. According to some such embodiments, the therapeutically effective amount of the HDAC inhibitor is about 90 mg/kg body weight. According to some such embodiments, the therapeutically effective amount of the HDAC inhibitor is about 100 mg/kg body weight. According to some such embodiments, the therapeutically effective amount of the HDAC inhibitor is about 110 mg/kg body weight. According to some such embodiments, the therapeutically effective amount of the HDAC inhibitor is about 120 mg/kg body weight. According to some such embodiments, the therapeutically effective amount of the HDAC inhibitor is about 130 mg/kg body weight. According to some such embodiments, the therapeutically effective amount of the HDAC inhibitor is about 140 mg/kg body weight. According to some such embodiments, the therapeutically effective amount of the HDAC inhibitor is about 150 mg/kg body weight. According to some such embodiments, the therapeutically effective amount of the HDAC inhibitor is about 160 mg/kg body weight. According to some such embodiments, the therapeutically effective amount of the HDAC inhibitor is about 170 mg/kg body weight. According to some such embodiments, the therapeutically effective amount of the HDAC inhibitor is about 180 mg/kg body weight. According to some such embodiments, the therapeutically effective amount of the HDAC inhibitor is about 190 mg/kg body weight. According to some such embodiments, the therapeutically effective amount of the HDAC inhibitor is about 250 mg/kg body weight. According to some such embodiments, the therapeutically effective amount of the HDAC inhibitor is about 500 mg/kg body weight.

According to some embodiments, the therapeutic amount of the HDAC inhibitor is effective in achieving an inhibition of IC₅₀ value in vitro ranging from about 0.000001 μM to about 10 μM. According to some embodiments, the therapeutic amount of the HDAC inhibitor is effective in achieving an inhibition of IC₅₀ value in vitro ranging from about 0.000001 μM to about 0.00001 μM. According to some embodiments, the therapeutic amount of the HDAC inhibitor is effective in achieving an inhibition of IC₅₀ value in vitro ranging from about 0.00001 μM to about 0.0001 μM. According to some embodiments, the therapeutic amount of the HDAC inhibitor is effective in achieving an inhibition of IC₅₀ value in vitro ranging from about 0.0001 μM to about 0.001 μM. According to some embodiments, the therapeutic amount amount of the HDAC inhibitor is effective in achieving an inhibition of IC₅₀ value in vitro ranging from about 0.001 μM to about 0.01 μM. According to some embodiments, the therapeutic amount of the HDAC inhibitor is effective in achieving an inhibition of IC₅₀ value in vitro ranging from about 0.01 μM to about 0.1 μM. According to some embodiments, the therapeutic amount of the HDAC inhibitor is effective in achieving an inhibition of IC₅₀ value in vitro ranging from about 0.1 μM to about 1.0 μM. According to some embodiments, the therapeutic amount of the HDAC inhibitor is effective in achieving an inhibition of IC₅₀ value in vitro ranging from about 1.0 μM to about 10.0 μM.

According to some embodiments, the HDAC inhibitor is selective toward HDAC6. According to one embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 100. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 500. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 1,000. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 5,000. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 10,000. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 20,000. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 30,000.

According to some embodiments, the therapeutic amount of the HDAC inhibitor compound is capable of achieving a half-maximal dose response (EC50) value of acetylated tubulin obtained in cell ranging between 0.05 μM to 0.5 μM. According to some embodiments, the therapeutic amount of the HDAC inhibitor compound is capable of achieving a half-maximal dose response (EC50) value of acetylated tubulin obtained in cell ranging between 0.01 μM to 2.7 μM. According to one embodiment, the therapeutic amount of the HDAC inhibitor compound is capable of achieving a half-maximal dose response (EC50) value of acetylated tubulin obtained in cell is at least 0.01 μM. According to one embodiment, the therapeutic amount of the HDAC inhibitor compound is capable of achieving a half-maximal dose response (EC50) value of acetylated tubulin obtained in cell is at least 0.02 μM. According to one embodiment, the therapeutic amount of the HDAC inhibitor compound is capable of achieving a half-maximal dose response (EC50) value of acetylated tubulin obtained in cell is at least 0.03 μM. According to one embodiment, the therapeutic amount of the HDAC inhibitor compound is capable of achieving a half-maximal dose response (EC50) value of acetylated tubulin obtained in cell is at least 0.04 μM. According to one embodiment, the therapeutic amount of the HDAC inhibitor compound is capable of achieving a half-maximal dose response (EC50) value of acetylated tubulin obtained in cell is at least 0.05 μM.

According to one embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 2.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 4.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 6.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 8.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 10.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 15.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 20.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 25.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 30.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 35.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 40.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 45.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 55.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 60.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 65.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 70.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 75.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 80.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 85.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 90.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 95.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 100.0.

According to another embodiment, the composition is a pharmaceutical composition.

According to another embodiment, the composition further comprises at least one therapeutic agent. According to another embodiment, the additional therapeutic agent is of a therapeutically effective amount.

### Method of Treating an HDAC-Associated Disease

According to another aspect, the present disclosure provides method of treating a histone deacetylase (HDAC)-associated disease, comprising

(a) providing at least one HDAC inhibitor of Formula I:

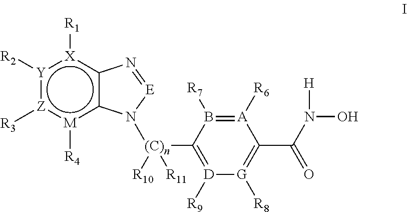

or a pharmaceutically acceptable salt thereof, and a carrier, wherein:

each of R₁, R₂, R₃ and R₄ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₁, R₂, R₃ and R₄ is H or a substituent when X, Y, Z and M is carbon;

E is C—R₅, or N;

R₅ is H, OH, NH₂, amino optionally substituted by alkyl or aryl, CN, F, Cl, Br, I, C₁-C₆ perfluoroalkyl, O-alkyl, O-aryl, O-heteroaryl, NO₂, cycloalkyl, aryl, acyl, optionally substituted C₁-C₆ alkyl, C₂-C₆ alkene, or C₂-C₆ alkyne, wherein when R₅ is OH, the compound exists as a keto tautomer, as an enol tautomer or as a mixture of keto-enol tautomers;

each of A, B, D, and G is independently C or N;

each of R₆, R₇, R₈, and R₉ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₆, R₇, R₈ and R₉ is H or a substituent when A, B, D and G is carbon;

each of R₁₀ and R₁₁ is independently H, alkyl, or aryl, wherein (C)ₙ optionally is a chiral center, wherein (C)ₙ can exist as both R and S enantiomers, with the proviso that when R₁₀ is H, R₁₁ is alkyl or aryl; and when R₁₁ is H, R₁₀ is alkyl or aryl; and

n is 0, 1, 2, 3, 4, 5, 6, 7, 8, 9 or 10; and

(b) administering a composition comprising a therapeutic amount of the at least one HDAC inhibitor of formula I, wherein the therapeutic amount is effective to inhibit the activity of at least one HDAC isoform and in treating symptoms of the HDAC-associated disease.

According to some embodiments, the HDAC inhibitor is a compound of Formula Ia:

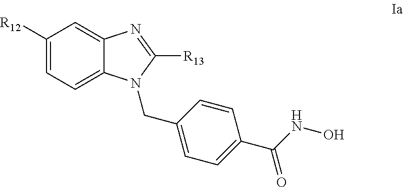

or a pharmaceutically acceptable salt thereof, and a carrier, wherein:

R₁₂ is H, alkyl, F, Cl, Br, I, or O-alkyl; and

R₁₃ is H or C₁-C₆ perfluoroalkyl; and a carrier.

According to some embodiments, the HDAC inhibitor is a compound of Ib:

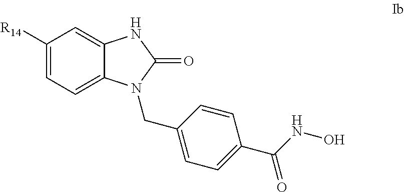

or a pharmaceutically acceptable salt thereof, and a carrier, wherein:

R₁₄ is H, alkyl, F, Cl, Br, I, O-alkyl, or C₁-C₆ perfluoroalkyl.

According to some embodiments, the HDAC inhibitor is a compound of Formula Ic:

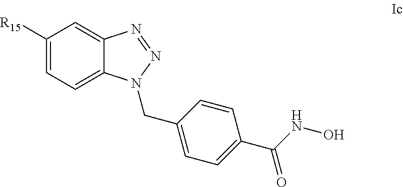

or a pharmaceutically acceptable salt thereof, and a carrier, wherein:

R₁₅ is H, alkyl, F, Cl, Br, I, or O-alkyl.

According to some embodiments, the HDAC inhibitor is selective toward HDAC6. According to one embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 100. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 500. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 1,000. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 5,000. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 10,000. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 20,000. According to another embodiment, a ratio of the inhibitory activity (IC₅₀) of the HDAC inhibitor obtained in vitro in the presence of an HDAC isoform selected from the group consisting of HDAC1, HDAC2, HDAC3, HDAC4, HDAC5, HDAC7, HDAC8, HDAC9, to the inhibition activity (IC₅₀) value of the HDAC inhibitor selective toward HDAC6 obtained in vitro in the presence of HDAC6 (in vitro selectivity value) has a value of at least 30,000.

According to some embodiments, the therapeutic amount of the HDAC inhibitor compound is capable of achieving a half-maximal dose response (EC50) value of acetylated tubulin obtained in cell ranging between 0.05 μM to 0.5 μM. According to some embodiments, the therapeutic amount of the HDAC inhibitor compound is capable of achieving a half-maximal dose response (EC50) value of acetylated tubulin obtained in cell ranging between 0.01 μM to 2.7 μM. According to one embodiment, the therapeutic amount of the HDAC inhibitor compound is capable of achieving a half-maximal dose response (EC50) value of acetylated tubulin obtained in cell is at least 0.01 μM. According to one embodiment, the therapeutic amount of the HDAC inhibitor compound is capable of achieving a half-maximal dose response (EC50) value of acetylated tubulin obtained in cell is at least 0.02 μM. According to one embodiment, the therapeutic amount of the HDAC inhibitor compound is capable of achieving a half-maximal dose response (EC50) value of acetylated tubulin obtained in cell is at least 0.03 μM. According to one embodiment, the therapeutic amount of the HDAC inhibitor compound is capable of achieving a half-maximal dose response (EC50) value of acetylated tubulin obtained in cell is at least 0.04 μM. According to one embodiment, the therapeutic amount of the HDAC inhibitor compound is capable of achieving a half-maximal dose response (EC50) value of acetylated tubulin obtained in cell is at least 0.05 μM.

According to one embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 2.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 4.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 6.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 8.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 10.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 15.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 20.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 25.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 30.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 35.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 40.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 45.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 55.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 60.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 65.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 70.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 75.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 80.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 85.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 90.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 95.0. According to another embodiment, a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 100.0.

HDACs have been associated with the pathology of a number of diseases such that inhibition of HDAC activity may be used to treat such diseases. Nonlimiting examples of indications that can be treated with HDAC inhibitors of the present application are described herein. Additional diseases beyond those disclosed herein may be later identified as being associated with HDACs. HDAC inhibitors of the described invention may be used to treat all such diseases.

Initial screening of the library of compounds against HDAC isoforms (using the protocol described in Bradner J E, West N, Grachan M L, Greenberg E F, Haggarty S J, Warnow T, Mazitschek R. Chemical phylogenetics of histone deacetylases. Nature Chemical Biology. 2010; 6: 238-243, which is incorporated herein in its entirety) have shown that these molecules are active in a 1 pM (picomolar)—10 μM (micromolar) concentration range towards HDAC6 isoforms. Because of the high selectivity profile of the compounds of the present invention against HDAC6 isoform, the HDAC inhibitors of the described invention are capable of being less toxic than currently available non-selective HDAC inhibitors, and thus can be selective towards specific cancers.

According to some embodiments, the HDAC-associated disease is selected from the group consisting of a cell proliferative disease, an autoimmune or inflammatory disorder and a neurodegenerative disease.

According to some embodiments, the HDAC-associated disease is characterized by lower level of acetylated tubulin in cells isolated from the subject with symptoms of the HDAC-associated disease relative to the level of acetylated tubulin in cells isolated from a healthy subject.

### Cell Proliferative Diseases

According to one embodiment, the HDAC associated disease is a cell proliferative disease. Such diseases include, but are not limited to, benign tumors, various types of cancers (such as with primary and metastasizing tumors), fibrotic diseases (such as, restenosis (such as coronary, carotid, and cerebral lesions), atherosclerosis, abnormal wound healing, abnormal angiogenesis, proliferative diseases associated with tissues with low levels of vaculature, and proliferative responses associated with organ transplants.

According to some embodiments, the method of treating an HDAC-associated disease achieves an inhibition of tumor growth. According to some embodiments, the method of treating an HDAC-associated disease achieves a reduction in number of viable cancer cells. According to some embodiments, the method of treating an HDAC-associated disease achieves an inhibition of tumor cell motility

According to some embodiments, HDAC inhibitors may be used in combination with other agents useful in treating cell proliferative diseases. According to some such embodiments, the agent is an anti-cell proliferation agent. Such anti-cell proliferation agents include, but are not limited to, retinoid acid and derivatives thereof, 2-methoxyestradiol, ANGIOSTATIN™ protein, ENDOSTATIN™ protein, suramin, squalamine, tissue inhibitor of metalloproteinase-I, tissue inhibitor of metalloproteinase-2, plasminogen activator inhibitor-1, plasminogen activator inhibitor-2, cartilage-derived inhibitor, paclitaxel, platelet factor 4, protamine sulfate (clupeine), sulfated chitin derivatives (prepared from queen crab shells), sulfated polysaccharide peptidoglycan complex (sp-pg), staurosporine, modulators of matrix metabolism, including for example, proline analogs ((I-azetidine-2-carboxylic acid (LACA), cishydroxyproline, d, 1-3, 4-dehydroproline, thiaproline), beta.-aminopropionitrile fumarate, 4-propyl-5-(4-pyridinyl)-2 (3H)-oxazolone; methotrexate, mitoxantrone, heparin, interferons, 2 macroglobulin-serum, chimp-3, chymostatin, beta-cyclodextrin tetradecasulfate, eponemycin; fumagillin, gold sodium thiomalate, d-penicillamine (CDPT), beta-1-anticollagenase-serum, alpha-2-antiplasmin, bisantrene, lobenzarit disodium, n-(2-carboxyphenyl-4-chloroanthronilic acid disodium or“CCA”, thalidomide; angostatic steroid, carboxyaminoimidazole; and metalloproteinase inhibitors such as BB94. Other agents that may be used include antibodies, for example monoclonal antibodies against angiogenic growth factors, e.g., bFGF, aFGF, FGF-5, VEGF isoforms, VEGF-C, HGF/SF and Ang-1/Ang-2. Ferrara N. and Alitalo, K. “Clinical application of angiogenic growth factors and their inhibitors” Nature Medicine 5: 1359-1364 (1999).

According to some embodiments, the HDAC-associated disease is a benign tumor disease. Such benign tumor disease that may be treated with HDAC inhibitors of the described invention may include, without limitation, hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

According to some embodiments, the HDAC-associated disease is a malignant tumor disease.

In one embodiment, the cell proliferative disease is a cancer. According to one embodiment, the cancer is primary or secondary. Exemplary cancers that may be treated with provided HDAC inhibitors include, but are not limited to, ovarian cancer, prostate cancer, lung cancer, acute myeloid leukemia, multiple myeloma, bladder carcinoma, renal carcinoma, breast carcinoma, colorectal carcinoma, neuroblastoma, melanoma, and gastric cancer.

According to some embodiments, the HDAC-associated disease is a cell proliferative condition associated with wounds. Such conditions may include, but are not limited to, surgical wounds, such as keloid scarring associated with surgery.

According to some embodiments, the HDAC-associated disease is a cell proliferative condition associated with fibrotic tissue. Such conditions may include, but are not limited to, emphysema, renal fibrosis, diabetic nephropathy, cardiac hypotrophy and fibrosis, idiopathic pulmonary fibrosis, system sclerosis, and cystic fibrosis.

According to some embodiments, the HDAC-associated disease is a cell proliferative condition associated with organ rejection during organ transplant. Such conditions may include, but are not limited to, organ transplants such as of heart, lung, liver, kidney and other body organs.

According to some embodiments, the HDAC-associated disease is a cell proliferative condition associated with abnormal angiogenesis. Such conditions may include, but are not limited to, abnormal angiogenesis accompanying rheumatoid arthritis, ischemic-reperfusion related brain edema and injury, cortical ischemia, ovarian hyperplasia and hypervascularity, (polycystic ovary syndrome), endometriosis, psoriasis, diabetic retinopathy, and other ocular angiogenic diseases such as retinopathy of prematurity (retrolental fibroplastic), macular degeneration, corneal graft rejection, and neuroscular glaucoma.

### Autoimmune or Inflammatory Conditions

According to some embodiments, the HDAC-associated disease is an autoimmune or inflammatory disorder. Such disorders may include, but are not limited to, rheumatoid arthritis, psoriasis, inflammatory bowel disease, multiple sclerosis, systemic lupus erthematosus, airway hyperresponsiveness, Crohn's disease, ulcerative colitis, autoimmune or inflammatory conditions associated with organ transplants, and autoimmune or inflammatory conditions associated with microbial infections.

Such autoimmune or inflammatory disorders may involve G-protein pathways (e.g., purinergic receptor-mediated, etc.) or non G-protein pathways (e.g., PPAR-mediated, Toll-like receptor-mediated, and TNF-alpha receptor-mediated, etc.).

### Neurodegenerative Conditions

According to some embodiments, the HDAC-associated disease is a neurodegenerative condition. Such conditions may include, but are not limited to, cerebral ischemia, Huntington's disease, amyotrophic lateral sclerosis, spinal musclular atrophy, Parkinson's disease, Alzheimer's disease and other cognitive disorders.

### Equivalents

Unless defined otherwise, all technical and scientific terms used herein have the same meaning as commonly understood by one of ordinary skill in the art to which this invention belongs. Although any methods and materials similar or equivalent to those described herein also can be used in the practice or testing of the present invention, the preferred methods and materials are now described. All publications mentioned herein are incorporated herein by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto. Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

It must be noted that as used herein and in the appended claims, the singular forms “a”, “and”, and “the” include plural references unless the context clearly dictates otherwise. All technical and scientific terms used herein have the same meaning.

The publications discussed herein are provided solely for their disclosure prior to the filing date of the present application and are incorporated herein by reference in their entirety. Nothing herein is to be construed as an admission that the present invention is not entitled to antedate such publication by virtue of prior invention. Further, the dates of publication provided may be different from the actual publication dates which may need to be independently confirmed.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### HDAC Inhibition Assay

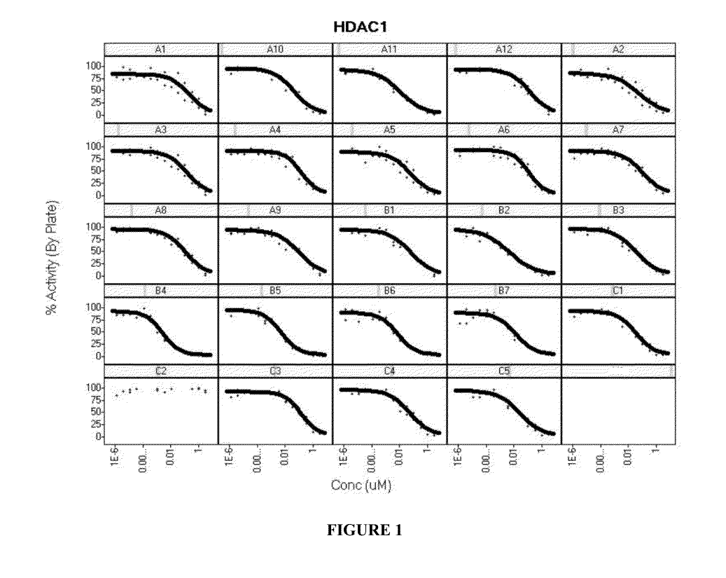

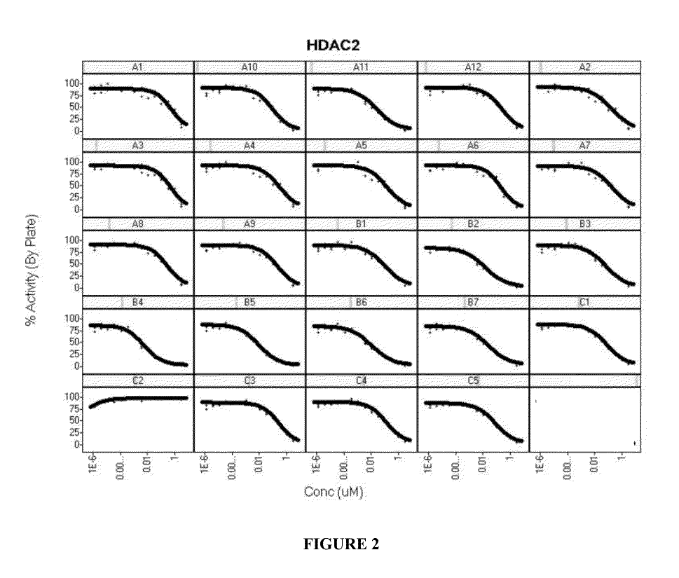

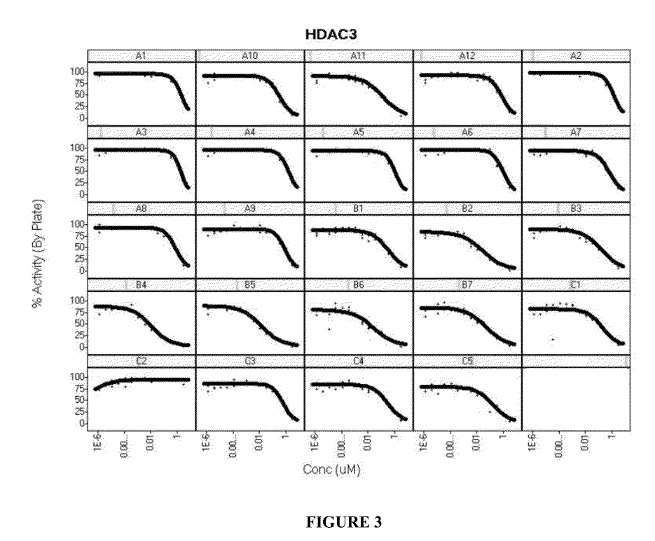

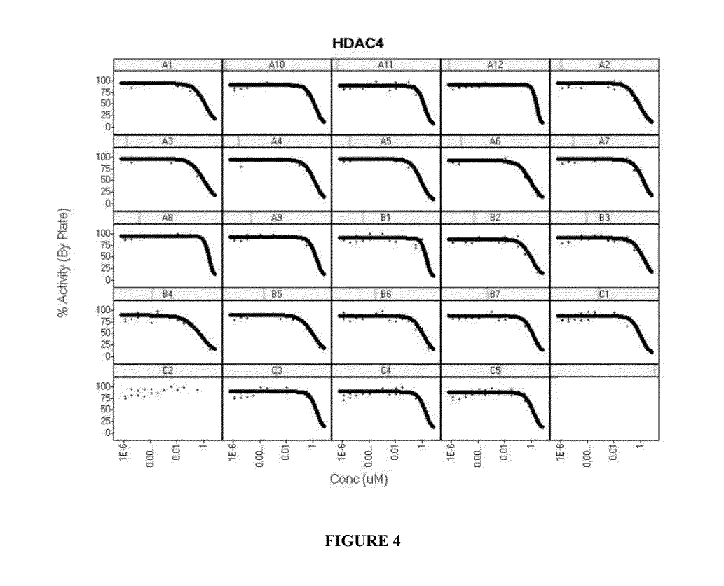

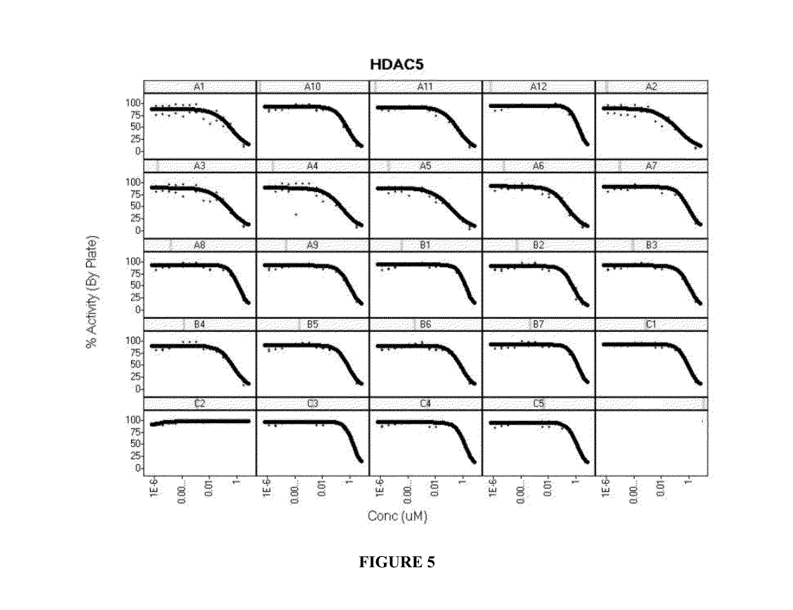

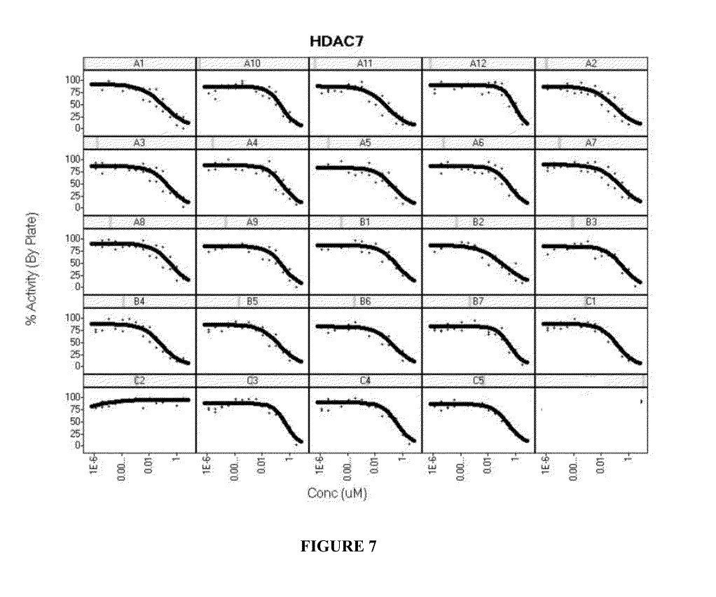

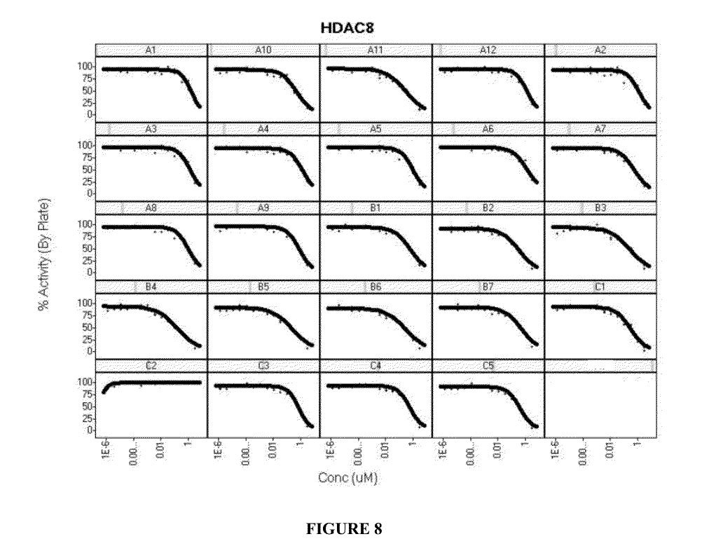

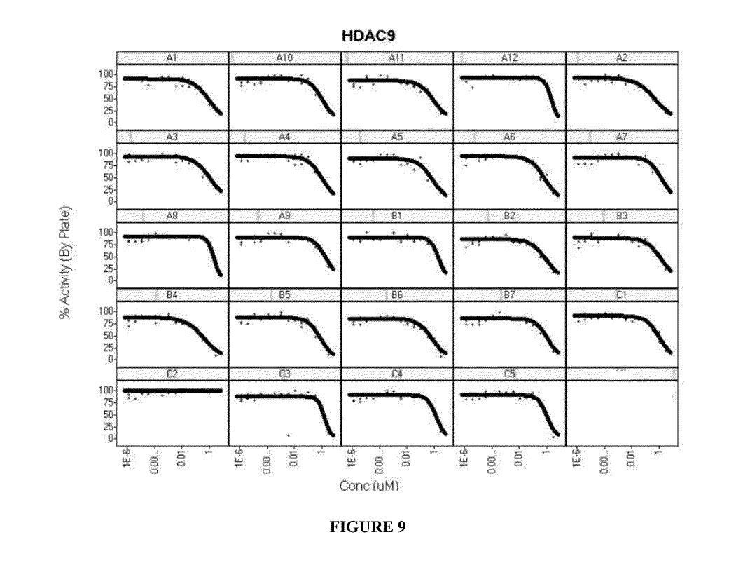

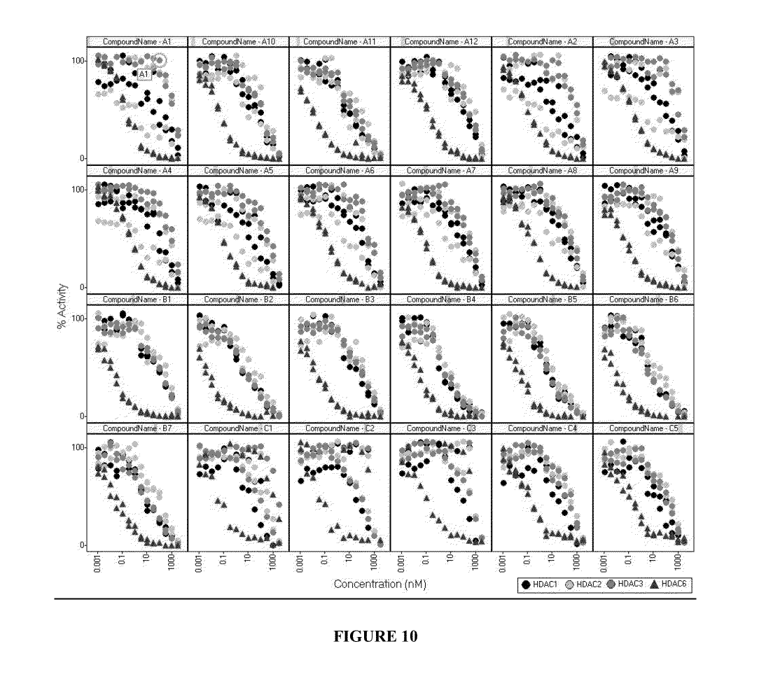

This Example shows the HDAC inhibitors of the described invention inhibit the activities of HDAC1, HDAC2, HDAC3, HDAC4, HDAC5, HDAC6, HDAC7, HDAC8 and HDAC9. The IC₅₀ values are listed in Tables 3-7. The dose response curves obtained with HDAC inhibitors and control drugs are shown in FIGS. 1-10.

In vitro Histone Deacetylase enzyme assays were performed using Class I (isoforms: HDAC1, HDAC2, HDAC3 and HDAC8), class IIa (isoforms: HDAC4, HDAC5, HDAC7 and HDAC9) and class IIb (isoform HDAC6).

The enzymatic activities of HDAC10 and HDAC11 are not yet determined with these or other prepared substrates. (Bradner J E, West N, Grachan M L, Greenberg E F, Haggarty S J, Warnow T, Mazitschek R. Chemical phylogenetics of histone deacetylases. Nature Chemical Biology. 2010; 6: 238-243).

HDAC activities were determined in vitro with an optimized homogenous assay performed in a 384-well plate. Recombinant, full-length HDAC protein (BPS Biosciences) was incubated with fluorophore conjugated substrate, MAZ1600 and MAZ1675, at Kₘ=[S]. (MAZ1600; 21 μM for HDAC1, 22 μM for HDAC2, 9 μM for HDAC3, 9 μM for HDAC6; MAZ1675; 10 μM for HDAC4, 40 μM for HDAC5, 22 μM for HDAC7, 282 μM for HDAC8, 26 μM for HDAC9). Reactions were performed in assay buffer (50 mM HEPES, 100 mM KCl, 0.001% Tween-20, 0.05% BSA, 200 μM tris(2-carboxyethyl)phosphine (TCEP), pH 7.4) and followed for fluorogenic release of 7-amino-4-methylcoumarin from substrate upon deacetylase and trypsin enzymatic activity. Fluorescence measurements were obtained every five minutes using a multilabel plate reader and plate-stacker (Envision; Perkin-Elmer). Each plate was analyzed by plate repeat, and the first derivative within the linear range was imported into analytical software (Spotfire DecisionSite and GraphPad Prism). Replicate experimental data from incubations with inhibitor were normalized to controls.

As shown in Tables 3-8 and FIGS. 1-10, initial screening of the library of compounds have shown promising results towards HDAC6 isoform selectivity in pm (picomolar) concentration range inhibition when compared to those of currently available HDAC inhibitors and drugs. Because of the high selectivity profile of the compounds of the present invention against HDAC6 isoform, it is expected that the HDAC inhibitors of the described invention are capable of being less toxic than currently available non-selective HDAC inhibitors, and would be selective towards specific cancers. These HDAC inhibitors therefore are promising as leads for a potential anti-cancer drug.

Table 3 below shows the 1050 values obtained with exemplary HDAC inhibitors of the described invention towards HDAC1 and HDAC2.

TABLE 3
Inhibition activity (IC₅₀) of HDAC1 and HDAC2 isoforms by the library of
HDAC inhibitor analogs (HDAC A1-A12, HDAC B1-B7 and HDAC C1-C5)

| | HDAC1 | | HDAC2 | |
|---|---|---|---|---|
| | IC₅₀ (μM) | SD | IC₅₀ (μM) | SD |
| A1 | 0.6821212811125 | 0.1603521512045 | 0.5811323325197 | 0.1309596517098 |
| A10 | 0.09649533745672 | 0.01596454282132 | 0.09414855531275 | 0.01693632898412 |
| A11 | 0.02874269854386 | 0.007557140808296 | 0.02667368216250 | 0.004551385175651 |
| A12 | 0.2987569159107 | 0.04431280027906 | 0.2440043176229 | 0.03797280018893 |
| A2 | 0.2470689496317 | 0.07740555301849 | 0.1691141436755 | 0.03886471497859 |
| A3 | 0.4660741316991 | 0.09216151680149 | 0.4844941608199 | 0.09158272310605 |
| A4 | 0.3975302340974 | 0.06453239041583 | 0.3004316579045 | 0.06412992994250 |
| A5 | 0.1674774490269 | 0.03886542659423 | 0.1459022624362 | 0.02933665393595 |
| A6 | 0.2462791830061 | 0.04246311181216 | 0.2046840944740 | 0.03474698133978 |
| A7 | 0.2636938671823 | 0.04034564983343 | 0.1813720895137 | 0.03384639037828 |
| A8 | 0.2622925276927 | 0.04218699386274 | 0.2902644287292 | 0.04318126468147 |
| A9 | 0.1864372263839 | 0.03257316785526 | 0.2075998111029 | 0.03471126701364 |
| B1 | 0.1403037307497 | 0.03672468899503 | 0.1012382141034 | 0.01897000485899 |
| B2 | 0.01323270804192 | 0.003512869115716 | 0.01430614165643 | 0.001862342414717 |
| B3 | 0.05927115345939 | 0.01395474223320 | 0.06473502550929 | 0.01204740612953 |
| B4 | 0.00528801637672 | 0.001381742077699 | 0.00530401063637 | 0.000875380925987 |
| B5 | 0.01040009149034 | 0.002557636178489 | 0.00908869997563 | 0.001294938170432 |
| B6 | 0.01210498310657 | 0.003231360860337 | 0.01088345766502 | 0.002098745088161 |
| B7 | 0.03151708323807 | 0.009559586818819 | 0.02550480418221 | 0.003901251475099 |
| C1 | 0.06390967853773 | 0.01952737325628 | 0.07811076832639 | 0.009140020145568 |
| C2 | — | | — | — |
| C3 | 0.2182100703290 | 0.06487261510389 | 0.2657378260915 | 0.03688134481976 |
| C4 | 0.09452183200053 | 0.03888174526831 | 0.1414430966421 | 0.02003773118696 |
| C5 | 0.05448298466739 | 0.01894306750158 | 0.08449990223432 | 0.009380378525529 |
| SAHA | 0.00168484130819 | 0.000658019220193 | 0.00108158097208 | 0.000183357976096 |

Table 4 below shows the IC₅₀ values obtained with exemplary HDAC inhibitors of the described invention towards HDAC3 and HDAC4.

TABLE 4
Inhibition activity (IC₅₀) of HDAC3 and HDAC4 isoforms by the library of
HDAC inhibitor analogs (HDAC A1-A12, HDAC B1-B7 and HDAC C1-C5)

| | HDAC3 | | HDAC4 | |
|---|---|---|---|---|
| | IC₅₀ (μM) | SD | IC₅₀ (μM) | SD |
| A1 | 1.790967440228 | 0.1378487949190 | 1.392615443589 | 0.1575491431530 |
| A10 | 0.2101785407622 | 0.04166521075760 | 1.484569361007 | 0.1677121564767 |
| A11 | 0.06559175302248 | 0.01307091814619 | 1.552789499649 | 0.1650246226425 |
| A12 | 0.4972826946030 | 0.08400893299566 | 2.693864415339 | 0.1353060887741 |
| A2 | 1.136374994444 | 0.1469747683034 | 0.8483431796265 | 0.1112855730109 |
| A3 | 1.794745519294 | 0.2114052177318 | 1.091099630371 | 0.1349261775349 |
| A4 | 1.269014412017 | 0.1499987774765 | 1.375039161187 | 0.1807398739231 |
| A5 | 0.7836017510088 | 0.1014321842781 | 0.8622070281862 | 0.06739958867946 |
| A6 | 0.8099157342808 | 0.1497062936944 | 0.9455497461935 | 0.1505074058457 |
| A7 | 0.4688582360920 | 0.06339054250715 | 1.757749674034 | 0.2637151385837 |
| A8 | 0.6079907182776 | 0.1033517143156 | 2.761121562453 | 0.1747231094849 |
| A9 | 0.6059154113492 | 0.09554474219438 | 1.905917120588 | 0.1970509441741 |
| B1 | 0.2115695854381 | 0.04568549779476 | 2.226566498640 | 0.2586069950985 |
| B2 | 0.01806720896423 | 0.004315728998806 | 1.182012621742 | 0.1611840515696 |
| B3 | 0.1359818948670 | 0.02633708678245 | 1.723330712278 | 0.2320361179595 |
| B4 | 0.008920457005940 | 0.001942882687986 | 0.6391112459778 | 0.1232517342058 |
| B5 | 0.01013435101107 | 0.001907901574258 | 1.182054906232 | 0.1253021617673 |
| B6 | 0.01468503378564 | 0.004469482269596 | 1.421411121399 | 0.2287584320995 |
| B7 | 0.03182551997606 | 0.008143331299028 | 1.544986807929 | 0.1405443846603 |
| C1 | 0.1643501836896 | 0.06881536620057 | 1.069196447436 | 0.1617749869470 |
| C2 | — | — | — | — |
| C3 | 0.5164670421108 | 0.08577718926030 | 2.131581673912 | 0.2728705281500 |
| C4 | 0.2164750638761 | 0.05104041977340 | 1.921971568138 | 0.2410357647237 |
| C5 | 0.1154779734197 | 0.03186914496797 | 1.555287819412 | 0.2049989284196 |
| SAHA | 0.002904403525140 | 0.0005097521465502 | — | — |

Table 5 below shows the IC₅₀ values obtained with exemplary HDAC inhibitors of the described invention towards HDAC5 and HDAC6.

TABLE 5
Inhibition activity (IC₅₀) of HDAC5 and HDAC6 isoforms by the library of
HDAC inhibitor analogs (HDAC A1-A12, HDAC B1-B7 and HDAC C1-C5)

| | HDAC5 | | HDAC6 | |
|---|---|---|---|---|
| | IC₅₀ (μM) | SD | IC₅₀ (μM) | SD |
| A1 | 0.450049168305 | 0.1433882516948 | 0.000472443216348 | 0.000104211082173 |
| A10 | 0.668337264005 | 0.0837786066883 | 0.000068999000662 | 0.0000152293903408 |
| A11 | 0.427380911741 | 0.0474869743150 | 0.000016662604281 | 0.0000035138506945 |
| A12 | 1.695616623403 | 0.1405459694312 | 0.000216853134742 | 0.0000358893921521 |
| A2 | 0.16403266027 | 0.050030681373 | 0.000305677888860 | 0.0000428324438911 |
| A3 | 0.278963086892 | 0.0763408201003 | 0.000574075982027 | 0.0000888600072707 |
| A4 | 0.285012156943 | 0.1123124541214 | 0.001157776674081 | 0.0001932118998361 |
| A5 | 0.147634383245 | 0.0305335724195 | 0.000309842372528 | 0.0000604564031579 |
| A6 | 0.249647145369 | 0.0419764352705 | 0.000391760417879 | 0.0000567931965958 |
| A7 | 0.958424720828 | 0.0972211402804 | 0.000097857573599 | 0.0000194108846970 |
| A8 | 1.404603445795 | 0.1325629435900 | 0.000380998284559 | 0.0000563737986148 |
| A9 | 1.017598477002 | 0.0901623639114 | 0.000066290953807 | 0.0000168320801515 |
| B1 | 1.711840034209 | 0.1347662779782 | 0.000014516129866 | 0.0000022871926234 |
| B2 | 0.658265313441 | 0.0797217771437 | 0.000002863150604 | 8.299373431663E-7 |
| B3 | 1.109073296836 | 0.1062349110494 | 0.000021561429316 | 0.0000036629424595 |
| B4 | 0.575751247463 | 0.0750429370864 | 0.000025260506558 | 0.0000037417405972 |
| B5 | 0.807521826281 | 0.0847157239243 | 0.000020490951506 | 0.0000031614310147 |
| B6 | 0.762165522076 | 0.0834553431261 | 0.000006205918382 | 0.0000012311813431 |
| B7 | 1.491412897552 | 0.1381266919627 | 0.000025747322879 | 0.0000045155773627 |
| C1 | 0.955861013736 | 0.0538930355362 | 0.000015991588180 | 0.0000026271756004 |
| C2 | — | — | 1.472038561353 | 0.2098275358578 |
| C3 | 1.805136501592 | 0.1135515755907 | 0.000026289078854 | 0.0000032122916179 |
| C4 | 1.455053407932 | 0.1170961801477 | 0.000055216443398 | 0.000007902065 |
| C5 | 1.256999474955 | 0.1070737765689 | 0.000025784244155 | 0.0000032722883233 |
| SAHA | — | — | 1.739060919142 | 0.3085928677040 |

Table 6 below shows the IC₅₀ values obtained with exemplary HDAC inhibitors of the described invention towards HDAC7 and HDAC8.

TABLE 6
Inhibition activity (IC₅₀) of HDAC7 and HDAC8 isoforms by the library of
HDAC inhibitor analogs (HDAC A1-A12, HDAC B1-B7 and HDAC C1-C5)

| | HDAC7 | | HDAC8 | |
|---|---|---|---|---|
| | IC₅₀ (μM) | SD | IC₅₀ (μM) | SD |
| A1 | 0.1205656575334 | 0.05489077486908 | 1.778061835295 | 0.2249197373295 |
| A10 | 0.2780265069642 | 0.07800875272268 | 0.5191793657651 | 0.07785570167502 |
| A11 | 0.07106133462910 | 0.02883035960306 | 0.3046772604865 | 0.04665534942523 |
| A12 | 0.8825884730388 | 0.2366159490751 | 1.418861493263 | 0.1413107173376 |
| A2 | 0.1063687412911 | 0.04975518595856 | 1.333942888320 | 0.2219364500598 |
| A3 | 0.2780757743029 | 0.1118570909545 | 1.458413782888 | 0.1740788120704 |
| A4 | 0.3741980992035 | 0.1237555499247 | 1.435493440706 | 0.1709743173183 |
| A5 | 0.2277999299838 | 0.09674789988848 | 1.084609182829 | 0.1337998507459 |
| A6 | 0.4144657865937 | 0.1305508817728 | 1.538536446569 | 0.2345842265175 |
| A7 | 0.2550422416813 | 0.09614716975257 | 0.6432265644249 | 0.07031727967786 |
| A8 | 0.4184660230077 | 0.1554237961540 | 1.084585062067 | 0.1278784283671 |
| A9 | 0.3265085696819 | 0.09253125545577 | 0.8185543098392 | 0.07627935584756 |
| B1 | 0.4428776810504 | 0.1251322425456 | 0.6725071361174 | 0.07963716248270 |
| B2 | 0.1594152507722 | 0.07357716113531 | 0.3458110394371 | 0.04163696001051 |
| B3 | 0.3670028980196 | 0.1131506133214 | 0.2781243650698 | 0.04964546621147 |
| B4 | 0.07964124254332 | 0.02791172199726 | 0.1685603307572 | 0.02759425936254 |
| B5 | 0.1904975360922 | 0.05704857068751 | 0.2593060566318 | 0.04230834626890 |
| B6 | 0.2372049096400 | 0.06208692719992 | 0.3018368056364 | 0.04894747139744 |
| B7 | 0.4102613240776 | 0.08600974702538 | 0.5510613744976 | 0.07150063602373 |
| C1 | 0.1484927943016 | 0.03119358348524 | 0.3054066597845 | 0.04273959399357 |
| C2 | — | — | — | — |
| C3 | 0.5553865873610 | 0.1110683009090 | 0.7589690620882 | 0.06709193853401 |
| C4 | 0.4200895895736 | 0.09238015812141 | 0.6146827735696 | 0.05686260437948 |
| C5 | 0.3046804952723 | 0.05099291081939 | 0.4249943131302 | 0.03850793215887 |
| SAHA | — | — | 1.655843204326 | 0.1540839030085 |

Table 7 below shows the IC₅₀ values obtained with exemplary HDAC inhibitors of the described invention towards HDAC9.

TABLE 7
Inhibition activity (IC₅₀) of HDAC9 isoform by the library of HDAC
inhibitor analogs (HDAC A1-A12, HDAC B1-B7 and HDAC C1-C5)

| | HDAC9 | |
|---|---|---|
| | IC₅₀ (μM) | SD |
| A1 | 1.023725410378 | 0.1957519117788 |
| A10 | 1.192186775784 | 0.2145108728713 |
| A11 | 1.018082951601 | 0.2102910319256 |
| A12 | 2.370849403047 | 0.2315482471325 |
| A2 | 0.5937378724875 | 0.09970992387581 |
| A3 | 1.221072893174 | 0.2266472468046 |
| A4 | 1.091436603281 | 0.1798287753643 |
| A5 | 0.7497235021610 | 0.1779667070610 |
| A6 | 0.7327960133745 | 0.1536967140537 |
| A7 | 1.627507327994 | 0.3448218358158 |
| A8 | 2.276407324523 | 0.2033975139930 |
| A9 | 2.186116420811 | 0.3676561127276 |
| B1 | 2.252700603546 | 0.2547368307899 |
| B2 | 0.9149268115423 | 0.1731834231979 |
| B3 | 1.443530373360 | 0.2933835684551 |
| B4 | 0.4161543284842 | 0.08657453993171 |
| B5 | 0.8089812532063 | 0.1500919826351 |
| B6 | 0.7116957986452 | 0.1190580240481 |
| B7 | 1.233802259145 | 0.1983527675494 |
| C1 | 1.084234393641 | 0.1484798877969 |
| C2 | — | — |
| C3 | 1.633554071536 | 0.5015248777202 |
| C4 | 1.437657542634 | 0.1783756138806 |
| C5 | 1.085704608266 | 0.1463953755456 |
| SAHA | — | — |

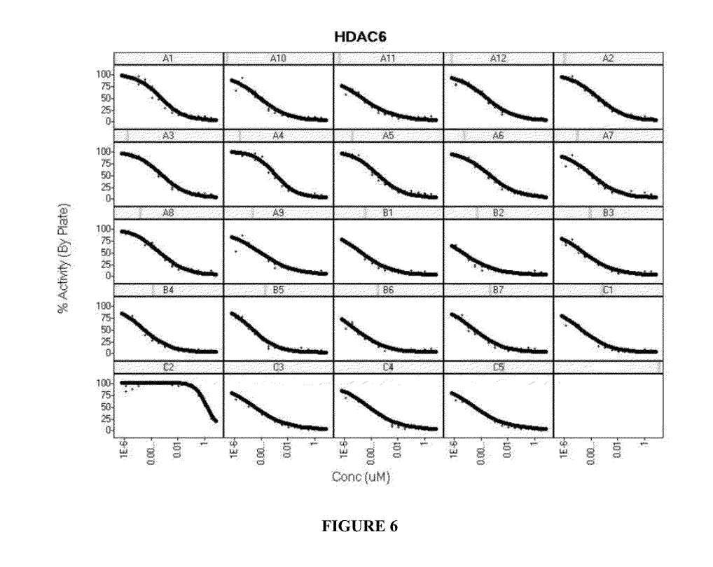

FIGS. 1-10 represent dose-response curves for inhibition of HDAC1, HDAC2, HDAC3, HDAC4, HDAC5, HDAC6, HDAC7, HDAC8 and HDAC9 obtained with HDAC inhibitors of the present invention, and show high selectivity and activity of the HDAC inhibitors towards the HDAC6 isoform. In the process of enzyme inhibition, the inhibitor binds to the active site of the enzyme. As shown in the HDAC6 curve (FIG. 6), the percent (%) activity of the enzyme decreases drastically as concentration of inhibitor is increased, because as the concentration of inhibitor is increased, binding of the inhibitor (drug) to the enzyme is increased eventually inhibiting the enzyme. For HDAC1, HDAC2, HDAC3, HDAC4, HDAC5, HDAC7, HDAC8 and HDAC9 as shown in the dose response curves (FIGS. 1-5 and FIGS. 7-9), the % activity of the enzyme remains constant (approximately 85%) as concentration of inhibitor is increased; eventually, the enzyme activity decreases only at very high inhibitor concentrations, because the inhibitor is not binding well to the active site of these enzyme isoforms; therefore it has less selectivity towards these isoforms. Therefore lower inhibition activity is observed with isoforms HDAC1, HDAC2, HDAC3, HDAC4, HDAC5, HDAC7, HDAC8 and HDAC9.

Table 8 demonstrates selectivity of HDAC inhibitors A1, A2, A3, A4, A5, A6, A7, A8, A9, A10, A11, A12, B1, B2, B3, B4, B5, B6, B7, C1, C2, C3, C4 and C5 toward HDAC6 isoform. The in vitro selectivity value for a given inhibitor is calculated as the ratio of IC₅₀ value obtained in vitro with the inhibitor for a given HDAC isoform relative to that of HDAC6. Inhibitors with in vitro selectivity values for HDAC6 of at least 100 are considered to have high selectivity toward HDAC6. Inhibitors with in vitro selectivity values for HDAC6 of at least 30,000 are considered to have exceptionally high selectivity toward HDAC6.

For example, HDAC inhibitor A11 has exceptionally high selectivity toward HDAC6 compared to HDAC4 and HDAC9 when tested in vitro.

HDAC inhibitor A9 as exceptionally high selectivity toward HDAC6 compared to HDAC9 when tested in vitro.

HDAC inhibitor B1 displays exceptionally high selectivity toward HDAC6 compared to HDAC4, HDAC5, HDAC7, HDAC8, and HDAC9 when tested in vitro.

HDAC inhibitor B2 displays exceptionally high selectivity toward HDAC6 compared to HDAC4, HDAC5, HDAC7, HDAC8, and HDAC9 when tested in vitro.

HDAC inhibitor B3 displays exceptionally high selectivity toward HDAC6 compared to HDAC4, HDAC5, and HDAC9 when tested in vitro.

HDAC inhibitor B5 displays exceptionally high selectivity toward HDAC6 compared to HDAC4, HDAC5, and HDAC9 when tested in vitro.

HDAC inhibitor B6 displays exceptionally high selectivity toward HDAC6 compared to HDAC4, HDAC5, and HDAC9 when tested in vitro.

HDAC inhibitor B7 displays exceptionally high selectivity toward HDAC6 compared to HDAC4, HDAC5, and HDAC9 when tested in vitro.

HDAC inhibitor Cl displays exceptionally high selectivity toward HDAC6 compared to HDAC4, HDAC5, and HDAC9 when tested in vitro.

HDAC inhibitor C3 displays exceptionally high selectivity toward HDAC6 compared to HDAC4, HDAC5, and HDAC9 when tested in vitro.

HDAC inhibitor C4 displays exceptionally high selectivity toward HDAC6 compared to HDAC4 when tested in vitro.

HDAC inhibitor C5 displays exceptionally high selectivity toward HDAC6 compared to HDAC4, HDAC5, and HDAC9 when tested in vitro.

SAHA, a canonical pan-inhibitor, showed no selectivity toward HDAC6 compared to the other HDAC isoforms.

TABLE 8
In vitro Selectivity of HDAC Inhibitors toward HDAC6

| Inhibit- | HDAC1/ | HDAC2/ | HDAC3/ | HDAC4/ | HDAC5/ | HDAC7/ | HDAC8/ | HDAC9/ |
|---|---|---|---|---|---|---|---|---|
| or | HDAC6 | HDAC6 | HDAC6 | HDAC6 | HDAC6 | HDAC6 | HDAC6 | HDAC6 |
| A1 | 1444 | 1230 | 3791 | 2948 | 953 | 255 | 3764 | 2167 |
| A2 | 808 | 553 | 3718 | 2775 | 537 | 348 | 4364 | 1942 |
| A3 | 812 | 844 | 3126 | 1901 | 486 | 484 | 2540 | 2127 |
| A4 | 343 | 259 | 1096 | 1188 | 246 | 323 | 1240 | 943 |
| A5 | 541 | 471 | 2529 | 2783 | 476 | 735 | 3501 | 2420 |
| A6 | 629 | 522 | 2067 | 2414 | 637 | 1058 | 3927 | 1871 |
| A7 | 2695 | 1853 | 4791 | 17962 | 9794 | 2606 | 6573 | 16631 |
| A8 | 688 | 762 | 1596 | 7247 | 3687 | 1098 | 2847 | 5975 |
| A9 | 2812 | 3132 | 9140 | 28751 | 15350 | 4925 | 12348 | 32978 |
| A10 | 1399 | 1364 | 3046 | 21516 | 9686 | 4029 | 7524 | 17278 |
| A11 | 1725 | 1601 | 3936 | 93190 | 25649 | 4265 | 18285 | 61100 |
| A12 | 1378 | 1125 | 2293 | 12423 | 7819 | 4070 | 6543 | 10933 |
| B1 | 9665 | 6974 | 14575 | 153386 | 117927 | 30509 | 46328 | 155186 |
| B2 | 4622 | 4997 | 6310 | 412836 | 229909 | 55678 | 120780 | 319552 |
| B3 | 2749 | 3002 | 6307 | 79927 | 51438 | 17021 | 12899 | 66950 |
| B4 | 209 | 210 | 353 | 25301 | 22793 | 3153 | 6673 | 16475 |
| B5 | 508 | 444 | 495 | 57687 | 39409 | 9297 | 12655 | 39480 |
| B6 | 1951 | 1754 | 2366 | 229041 | 122813 | 38222 | 48637 | 114680 |
| B7 | 1224 | 991 | 1236 | 60006 | 57925 | 15934 | 21403 | 47920 |
| C1 | 3996 | 4884 | 10277 | 66860 | 59773 | 9286 | 19098 | 67800 |
| C2 | | | | | | | | |
| C3 | 8300 | 10108 | 19646 | 81082 | 68665 | 21126 | 28870 | 62138 |
| C4 | 1712 | 2562 | 3920 | 34808 | 26352 | 7608 | 11132 | 26037 |
| C5 | 2113 | 3277 | 4479 | 60319 | 48751 | 11817 | 16483 | 42107 |
| SAHA | 0 | 0 | 0 | 0 | 0 | 0 | 1 | 0 |

### Example 2

### High-Content Image Analysis of Induction of Acetylated Histones Versus Acetylated Tubulin in Cultured Cancer Cells

Acetylated histone is an endogenous marker for HDAC1, HDAC2 and HDAC3, whereas acetylated tubulin is a marker of HDAC6 activity. This Example shows that exemplary HDACi compounds of the present invention, such as A1, A4 and B6, show greater selectivity for HDAC6 in comparison to HDAC1, HDAC2 and HDAC3 relative to the non-specific marker SAHA. For obtaining the dose response curves for half-maximal induction of acetylated histones or acetylated tubulin, A549 (adenocarcinomic human alveolar basal epithelial) cells were plated at 4,000 cells/well in 50 μL in 384-well clear bottom plates (Corning 3712) and incubated overnight. Cells were treated with each test HDACi compound with an automated pin transfer instrument (Janus, Perkin Elmer) and incubated for 8 hours. Following incubation with the test HDACi compound, medium was aspirated (EL406, BioTek), cells were fixed in 40 μL formaldehyde solution (3.7% formaldehyde in PBS) and incubated 20 minutes at 4° C. Fixation solution was aspirated and 90 μL of washing solution (0.1% Triton X-100 in PBS) was added prior to 10 minute incubation at 4° C. Washing solution was aspirated and cells were incubated at 4° C. for about 1 hour in blocking solution (0.1% Triton X-100+2% BSA in PBS). Washing solution was aspirated, and cells were incubated for about 1 hour at 4° C. in primary antibody for acetylated-tubulin (Sigma T7451) and acetylated-histone (Cell Signaling #9441L) at a 1:1000 dilution in blocking solution. Primary antibody solution was aspirated, and cells were washed three times in 90 μL of washing solution. Following the third wash, cells were incubated in 10 μL for 90 minutes at room temperature in secondary antibody (Invitrogen A-21202, A-21244) and nuclear staining (Invitrogen H3570) solution at a 1:500 and 1:1000 dilution, respectively, in blocking solution. Secondary antibody and nuclear staining solution was aspirated and cells were washed three times in 90 μL of washing solution. After the third wash, 50 μL of PBS was added to each well. Image acquisition was done on a high content imaging microscope (ImageXpress Micro, Molecular Devices), and image analysis (MetaXpress, Molecular Devices) was performed to obtain average acetylated-tubulin and acetylated-histone signal per cell based on treatment. Replicate experimental data from incubations with inhibitor were normalized to DMSO controls. Dose response data was generated (Graphpad Prism) by normalization of maximum and minimum acetylated-tubulin and acetylated-histone signal compared to control (SAHA).

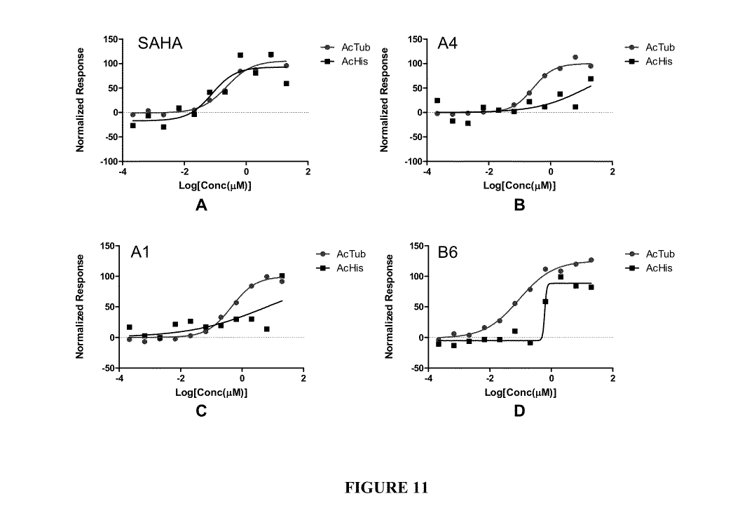

FIG. 11 shows plots of EC50 (μM) values obtained for half-maximal induction of acetylated histones (Squares) or acetylated tubulin (Circles) as measured by quantitative, automated epifluorescence microscopy, with a control compound, SAHA in (A), HDAC inhibitor A4 in (B), HDAC inhibitor A1 in (C), and HDAC inhibitor B6 in (D). Data are presented relative to a control compound, SAHA (vorinostat; Merck Research Laboratories), which is non-selective for nuclear deacetylases (HDAC1, HDAC2, HDAC3) and the tubulin deacetylase (HDAC6).

Table 9 lists EC50 (μM) values obtained for half-maximal induction of acetylated histones (AcHistone) or acetylated tubulin (AcTubulin) as measured by quantitative, automated epifluorescence microscopy, with HDACi A1, HDACi A2, HDACi A3, HDACi A4, HDACi A5, HDACi A6, HDACi A7, HDACi A8, HDACi A9, HDACi A10, HDACi A11, HDACi A12, HDACi B1, HDACi B2, HDACi B3, HDACi B4, HDACi B5, HDACi B6, HDACi B7, HDACi Cl, HDACi C2, HDACi C3, HDACi C4, or HDACi C5 relative to SAHA.

TABLE 9
EC50 (μM) values obtained for half-maximal induction of acetylated
histones (AcHistone) or acetylated tubulin (AcTubulin)

| Compound | AcHistone | AcTubulin |
|---|---|---|
| A1 | 6.337 | 0.4523 |
| A2 | 0.2418 | 0.1003 |
| A3 | 0.5924 | 0.0773 |
| A4 | 14.88 | 0.275 |
| A5 | 1.932 | 0.19 |
| A6 | 2.593 | 0.2124 |
| A7 | 0.2315 | 0.1587 |
| A8 | 0.07948 | 0.1047 |
| A9 | 0.06271 | 0.1316 |
| A10 | 0.9 | 0.188 |
| A11 | 1.971 | 0.1742 |
| A12 | 0.1048 | 0.2212 |
| B1 | 0.2628 | 0.08936 |
| B2 | 0.2289 | 0.09529 |
| B3 | 1.482 | 0.07024 |
| B4 | 0.4198 | 0.08047 |
| B5 | 0.8465 | 0.1323 |
| B6 | ~0.6293 | 0.04848 |
| B7 | 1.541 | 0.1539 |
| C1 | 7.142 | 2.704 |
| C2 | 2.515 | 0.1239 |
| C3 | 1.504 | 0.1439 |
| C4 | 6.036 | 1.924 |
| C5 | 2.299 | 0.1496 |
| SAHA | 0.143 | 0.2062 |

FIG. 11 and Table 9 show that the non-specific inhibitor SAHA is capable of inhibition of both nuclear deacetylases such as HDAC1, HDAC2 and HDAC3 as evidenced by the dose-dependent increase in the levels of both acetylated histone, marker for nuclear deacetylase inhibition, as well as inhibition of the tubulin-specific deacetylase HDAC6, as evidenced by the dose-dependent increase in acetylated tubulin (FIG. 11A). In contrast, exemplary HDACi compounds of the present invention, such as compounds A1, A4 and B6, show a dose-dependent increase in acetylated tubulin, but absence of the dose-dependent response on increased levels of acetylated histone, supporting the selective inhibition of the tubulin-specific deacetylase HDAC6 as compared to the nuclear deacetylases such as HDAC1, HDAC2 and HDAC3. (FIGS. 11B, 11C and 11D).

HDAC inhibitors of the present invention shows high in cell selectivity toward HDAC6 as evidenced by the high ratio of acetylated histone to acetylated tubulin.

TABLE 10
In cell Selectivity of
HDAC Inhibitors toward HDAC6

| Inhibitor | AcHistone/AcTubulin |
|---|---|
| A1 | 14.01061 |
| A2 | 2.410768 |
| A3 | 7.663648 |
| A4 | 54.10909 |
| A5 | 10.16842 |
| A6 | 12.2081 |
| A7 | 1.458727 |
| A8 | 0.759121 |
| A9 | 0.47652 |
| A10 | 4.787234 |
| A11 | 11.31458 |
| A12 | 0.473779 |
| B1 | 2.940913 |
| B2 | 2.402141 |
| B3 | 21.09909 |
| B4 | 5.216851 |
| B5 | 6.398337 |
| B6 | 12.98061 |
| B7 | 10.013 |
| C1 | 2.641272 |
| C2 | 20.29863 |
| C3 | 10.4517 |
| C4 | 3.137214 |
| C5 | 15.36765 |
| SAHA | 0.693501 |

### Example 3

### Cell Viability and Proliferation Assays

The effect of exemplary HDACi compounds of the present invention on proliferation of cancer cell lines can be assessed by measuring increase in dye absorbance of 3-(4,5-dimethylthiazol-2-yl)-2,5 diphenyl tetrasodium bromide (MTT) as an indicator of proliferation, as described in Mosmann T., “Rapid colorimetric assay for cellular growth and survival. Application to proliferation and cytotoxicity assays.” J. Immunol. Methods,” 1983, 16; 65 (1-2):55-63; Santo, L. et al., “A novel small molecule multi-cyclin-dependent kinase inhibitor, induces apoptosis in multiple myeloma via GSK-3 beta activayion and RNA polymerase II inhibition,” Oncogene, 2010, 29(16): 2325-2336; Santo L. et al., “Preclinical activity, pharmacodynamic and pharmacokinetic properties of a selective HDAC6 inhibitor, ACY-1215, in combination with bortezomib in multiple myeloma,” Blood, 2012, 119(11): 2579-89, the contents of which are incorporated by reference herein.

Exemplary cells that can be used include, but are not limited to, multiple myeloma (MM) cell lines (e.g. dexamethasone (Dex) sensitive (MM.1S) and Dex resistant (MM.1R) human MM cell lines, RPMI8226, U266 human MM cell lines, melphalan-resistant RPMI-LR5 (LR5) and doxorubicin-resistant RPMI_Dox40 (Dox40) cell lines, OPMI1 cells, ANBL-6 bortezomib-resistant (ANBL-6.BR) cells, fresh peripheral blood mononuclear cells (PBMNCs) obtained from multiple myeloma patients as well as healthy volunteers as control. An exemplary cell line is incubated with different concentrations of each test HDACi compound of the present invention for about 5-10 hours. Following incubation with each test HDACi compound, MTT solution is added to each sample and incubated at 37° C. for about 4 hours. The MTT assay involves the conversion of the water soluble MTT (3-(4,5-dimethylthiazol-2-yl)-2,5-diphenyltetrazolium bromide) to an insoluble formazan. The formazan is then solubilized, and the concentration determined by optical density at 570 nm. A dose-response curve as to the affect of each test HDACi compound on cell proliferation can then be obtained by normalization against a control.

Cell viability can be assessed with an exemplary viable stain such as Alamar Blue, Evans blue, TUNEL assay, etc.

### Example 4

### Detection of Apoptosis

Exemplary cells, such as multiple myeloma (MM) cell lines (e.g. dexamethasone (Dex) sensitive (MM.1S) and Dex resistant (MM.1R) human MM cell lines, RPMI8226, U266 human MM cell lines, melphalan-resistant RPMI-LR5 (LR5) and doxorubicin-resistant RPMI_Dox40 (Dox40) cell lines, OPMI1 cells, ANBL-6 bortezomib-resistant (ANBL-6.BR) cells, are cultured for about 24 hours with or without different concentrations of test HDACi compounds of the present invention. The cells are harvested, washed and stained with Annexin V/PI, as described in Raje N. et al., “Preclinical activity of P276-00, a novel small-molecule cyclin-dependent kinase inhibitor in the therapy of multiple myeloma,” Leukemia, 2009, 23(5): 961-970; Santo L. et al., “Preclinical activity, pharmacodynamic and pharmacokinetic properties of a selective HDAC6 inhibitor, ACY-1215, in combination with bortezomib in multiple myeloma,” Blood, 2012, 119(11): 2579-89, the contents of which are incorporated by reference herein. The number of Annexin V+PI-apoptotic cells can be counted using a flow cytometer.

### Example 5

### Immunofluorescence Assay

Cancer cell lines, such as MM.1S cells, can be cultured on tissue culture medium treated glass slides with or without different concentrations of test HDACi compounds of the present invention at about 1 μm to about 10 μm, as described in Santo L. et al., “Preclinical activity, pharmacodynamic and pharmacokinetic properties of a selective HDAC6 inhibitor, ACY-1215, in combination with bortezomib in multiple myeloma,” Blood, 2012, 119(11): 2579-89, which is incorporated herein by reference. The cells are fixed, permeabilized, blocked and stained with anti-ubiquitin antibody. The cells are then washed and incubated with a secondary antibody, such as Alexa-fluor goat anti-mouse antibody. Following subsequent washes and Hoechst staining, slides are mounted and images taken with a fluorescence microscope.

### Example 6

### Multiple Myeloma Mouse Model (Plasmacytoma Xenograft Model)

To induce multiple myeloma, male SCID mice are inoculated with exemplary multiple myeloma cells in a serum free medium, as described in Santo L. et al., “Preclinical activity, pharmacodynamic and pharmacokinetic properties of a selective HDAC6 inhibitor, ACY-1215, in combination with bortezomib in multiple myeloma,” Blood, 2012, 119(11): 2579-89, which is incorporated by reference herein After the tumors have reached a measurable size, the mice are treated with increasing concentrations of test HDACi compounds intraperitoneally at about 0.5 mg/kg to about 50 mg/kg, once daily for about 2-3 weeks. A control group receives the carrier alone according to an identical regimen as the test group. Tumor size and volume are measured and recorded daily. The mice are euthanized once the tumor size reaches about 2 cm³ or ulcerated.

For pharmacokinetic and pharmacodynamic studies, the mice are treated with increasing concentrations of test HDACi compounds orally, or intraperitoneally at about 0.5 mg/kg to about 50 mg/kg, once daily for about 2-3 weeks, and a control group with the carrier alone, after the tumors have reached about 150-200 mm³. The mice are then euthanized at predetermined time points, such as at 1 hour, at 6 hours, and at 24 hours after treatment. Tumors and blood are collected from each animal for immunohistochemistry, Western blot and flow cytometry.

### Example 7

### Disseminated Multiple Myeloma Model

Female SCID-beige mice are inoculated with multiple myeloma cells to induce disseminated multiple myeloma with metastases of small size, as described in Santo L. et al., “Preclinical activity, pharmacodynamic and pharmacokinetic properties of a selective HDAC6 inhibitor, ACY-1215, in combination with bortezomib in multiple myeloma,” Blood, 2012, 119(11): 2579-89, which is incorporated by reference herein. After the tumors have reached a measurable size, the mice are treated with increasing concentrations of test HDACi compounds intraperitoneally at about 0.5 mg/kg to about 50 mg/kg, once daily for about 2-3 weeks. A control group receives the carrier alone according to an identical regimen as the test group. Bioluminescence imaging is performed prior to and weekly upon treatment to follow disease progression.

### Example 8

### Biolumiscent Multiple Myeloma Model

To evaluate the effect of test HDACi compounds of the present invention in vivo, a bioluminescent multiple myeloma model (MM.1S-luc) can be used, such as described in Mitsiades, C. S. et al, “Inhibition of the insulin-like growth factor receptor-1 tyrosine kinase activity as a therapeutic strategy for multiple myeloma, other hematologic malignancies, and solid tumors,” Cancer Cell, 2004, 5: 221-230; and Delmore, J. E. et al., “BET bromodomain inhibition as a therapeutic strategy to target c-Myc,” Cell, 2011, 146: 904-917, each of which is incorporated by reference herein The tumor-bearing mice are treated with increasing concentrations of test HDACi compounds intraperitoneally at about 0.5 mg/kg to about 50 mg/kg, once daily for about 2-3 weeks. A control group receives the carrier alone according to an identical regimen as the test group. Tumor size and volume are measured and recorded daily.

### Example 9

### Vk*MYC Multiple Myeloma Mouse Model

To evaluate the effect of test HDACi compounds of the present invention in vivo, a Vk*MYC mouse model can be used, such as described in Chesi, M. et al., “AID-dependent activation of a MYC transgene induces multiple myeloma in a conditional mouse model of post-germinal center malignancies,” Cancer Cell, 2008, 13(2): 167-180; Keats, J. J. et al., “Clonal competition with alternating dominance in multiple myeloma,” Blood, 2012, Published online Apr. 12, 2012, Epub ahead of print, doi: 10.1182/blood-2012-01-405985, pages 1-27; and Chesi, M. et al., “Drug response in a genetically engineered mouse model of multiple myeloma is predictive of clinical efficacy,” Blood, 2012, published online on Mar. 26, 2012, Epub ahead of print, doi: 10.1182/blood-2012-02-412783, pages 1-30, each of which is incorporated by reference herein. The Vk*-MYC mice have characteristic genetic rearrangements of MYC gene and undergo a progression of monoclonal gammopathy to multiple myeloma. The disease progression is induced by introducing the Vk*-MYC transgene into a strain of mice, such as C57B1/6. The multiple myeloma cells in Vk*-MM mice secrete a high level of serum monoclonal antibody, termed an M-spike, that is detected using serum protein electrophoresis, as described in Chesi, M. et al., “AID-dependent activation of a MYC transgene induces multiple myeloma in a conditional mouse model of post-germinal center malignancies,” Cancer Cell, 2008, 13(2): 167-180; and Chesi, M. et al., “Drug response in a genetically engineered mouse model of multiple myeloma is predictive of clinical efficacy,” Blood, 2012, published online on Mar. 26, 2012, Epub ahead of print, doi: 10.1182/blood-2012-02-412783, pages 1-30, each of which is incorporated by reference herein. Vk*-MYC mice with established multiple myeloma are treated with increasing concentrations of test HDACi compounds intraperitoneally at about 0.5 mg/kg to about 50 mg/kg, once daily for about 2-3 weeks. A control group receives the carrier alone according to an identical regimen as the test group. Tumor size and volume are measured and recorded daily.

### Example 10

### Dextran Sodium Sulfate (DSS) and Adoptive Transfer Colitis Mouse Model

To evaluate the effect of test HDACi compounds of the present invention, dextran sodium sulfate (DSS) and an adoptive transfer colitis mouse model can be used as described in Wirtz, S. et al., “Mouse models of inflammatory bowel disease,” Adv. Drug Deliv. Rev., 2007, 59: 1073-1083; and de Zoeten, E. F. et al., “Histone deacetylase 6 and heat shock protein 90 control the functions of Foxp3+T-regulatory cells,” Mol. Cell. Biol., 2011, 31(10): 2066-2078, each of which is incorporated by reference herein. Wild-type B6 mice are given freshly prepared 4% (wt/vol) DSS in tap water for 7 days with tubacin or niltubacin to induce colitis. The colitis mice are treated with increasing concentrations of test HDACi compounds intraperitoneally at about 0.5 mg/kg to about 50 mg/kg, once daily for about 2-3 weeks. A control group receives the carrier alone according to an identical regimen as the test group. Effect on colitis is monitored by stool consistency and fecal blood. A T-cell dependent model can also be used, as described in Mudter, J. et al., “A new model of chronic colitis in SCID mice induced by adoptive transfer of CD62L+CD4+T cells: insights into the regulatory role of interleukin-6 on apoptosis,” Pathobiology, 2002, 70: 170-176; and de Zoeten, E. F. et al., “Histone deacetylase 6 and heat shock protein 90 control the functions of Foxp3+T-regulatory cells,” Mol. Cell. Biol., 2011, 31(10): 2066-2078.

The present invention may be embodied in other specific forms without departing from the spirit or essential attributes thereof and, accordingly, reference should be made to the appended claims, rather than to the foregoing specification, as indicating the scope of the invention.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

1. A compound of Formula I: 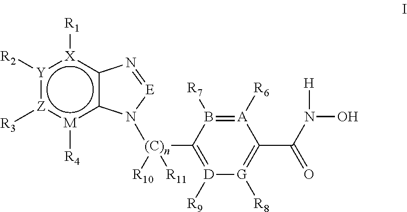
or a pharmaceutically acceptable salt thereof, wherein:
each of X, Y, Z and M is independently C or N;
each of R₁, R₂, R₃ and R₄ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₁, R₂, R₃ and R₄ is H or a substituent when X, Y, Z and M is carbon;
E is C—R₅, or N;
R₅ is H, OH, NH₂, amino optionally substituted by alkyl or aryl, CN, F, Cl, Br, I, C₁-C₆ perfluoroalkyl, O-alkyl, O-aryl, O-heteroaryl, NO₂, cycloalkyl, aryl, acyl, optionally substituted C₁-C₆ alkyl, C₂-C₆ alkene, or C₂-C₆ alkyne, wherein when R₅ is OH, the compound exists as a keto tautomer, as an enol tautomer or as a mixture of keto-enol tautomers;
each of A, B, D, and G is independently C or N;
each of R₆, R₇, R₈, and R₉ is independently H, OH, NH₂, amino optionally substituted by alkyl or aryl, carboxy, carbamoyl optionally substituted by alkyl, aminosulfonyl optionally substituted by alkyl, silyloxy optionally substituted by alkoxy, alkyl, or aryl, silyl optionally substituted by alkoxy, CN, F, Cl, Br, I, C₁-C₆ perfluoroalkyl, O-alkyl, O-aryl, O-heteroaryl, NO₂, cycloalkyl, aryl, acyl, mercapto, oxo, carboxy, optionally substituted C₁-C₆ alkyl, C₂-C₆ alkene, or C₂-C₆ alkyne, with the-proviso that R₆, R₇, R₈ and R₉ is H or a substituent when A, B, D and G is carbon;
each of R₁₀ and R₁₁ is independently H, alkyl, or aryl, wherein (C)ₙ optionally is a chiral center, wherein (C)ₙ can exist as both R and S enantiomers, with the proviso that when R₁₀ is H, R₁₁ is alkyl or aryl; and when R₁₁ is H, R₁₀ is alkyl or aryl; and
n is 0, 1, 2, 3, 4, 5, 6, 7, 8, 9 or 10,
wherein the compound is a histone deacetylatase (HDAC) inhibitor, and wherein the HDAC inhibitor inhibits histone deacetylating activity of at least one HDAC isoform selected from the group consisting of HDAC1, HDAC2, HDAC3, HDAC4, HDAC5, HDAC6, HDAC7, HDAC8, HDAC9, and a combination thereof.

2. The compound according to claim 1, wherein the compound of formula I is a compound of Formula Ia: 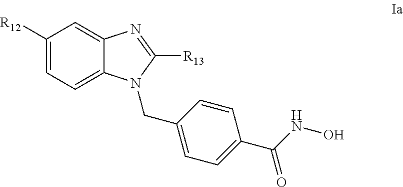
or a pharmaceutically acceptable salt thereof, wherein:
R₁₂ is selected from the group consisting of H, alkyl, F, Cl, Br, I, and O-alkyl; and
R₁₃ is selected from the group consisting of H and C₁-C₆ perfluoroalkyl.

3. The compound according to claim 1, wherein the compound of Formula I is a compound of Formula Ib: 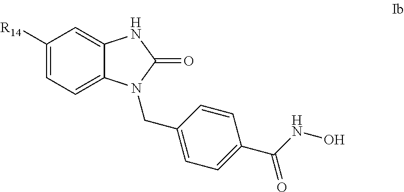
or a pharmaceutically acceptable salt thereof, wherein:
R₁₄ is selected from the group consisting of H, alkyl, F, Cl, Br, I, O-alkyl, and C₁-C₆ perfluoroalkyl.

4. The compound according to claim 1, wherein the compound of Formula I is a compound of Formula Ic: 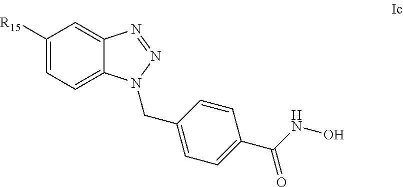
or a pharmaceutically acceptable salt thereof, wherein:
R₁₅ is selected from the group consisting of H, alkyl, F, Cl, Br, I, and O-alkyl.

5. The compound according to claim 1, wherein the HDAC inhibitor inhibits the histone deacetylating activity of at least one HDAC isoform with an inhibition activity (IC₅₀) from about 0.005 μM to about 2.76 μM.

6. The compound according to claim 1, wherein the HDAC inhibitor inhibits the histone deacetylating activity of HDAC6 with an inhibition activity (IC₅₀) from about 0.000001 μM to about 0.001 μM.

7. The compound according to claim 1, wherein the HDAC inhibitor is selective toward HDAC6.

8. The compound according to claim 7, wherein a ratio of the inhibitory activity (IC₅₀) of the HDAC inhibitor obtained in the presence of an HDAC isoform selected from the group consisting of HDAC1, HDAC2, HDAC3, HDAC4, HDAC5, HDAC7, HDAC8, and HDAC9, to the inhibition activity (IC₅₀) value of the HDAC inhibitor selective toward HDAC6 obtained in vitro in the presence of HDAC6 (in vitro selectivity value) has a value of at least 100.

9. The compound according to claim 7, wherein a ratio of the inhibitory activity (IC₅₀) of the HDAC inhibitor obtained in the presence of an HDAC isoform selected from the group consisting of HDAC1, HDAC2, HDAC3, HDAC4, HDAC5, HDAC7, HDAC8, and HDAC9, to the inhibition activity (IC₅₀) value of the HDAC inhibitor selective toward HDAC6 obtained in vitro in the presence of HDAC6 (in vitro selectivity value) has a value of at least 30,000.

10. The compound according to claim 7, wherein a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 2.0.

11. The compound according to claim 7, wherein a ratio of the half-maximal dose response (EC50) value of acetylated histone obtained in cell with a HDAC inhibitor to the half-maximal dose response (EC50) value of acetylated tubulin obtained in cell with the HDAC inhibitor (in cell selectivity value) has a value of at least 50.0.

12-36. (canceled)

37. A composition for treating a histone deacetylase (HDAC)-associated disease, wherein the composition comprises
(a) at least one compound of Formula I according to claim 1; and
(b) a pharmaceutically acceptable carrier.

38-40. (canceled)

41. The composition according to claim 37, wherein the HDAC inhibitor compound inhibits the histone deacetylating activity of at least one HDAC isoform with an inhibition activity (IC₅₀) of from about 0.005 μM to about 2.76 μM.

42. The composition according to claim 37, wherein the HDAC inhibitor compound inhibits the histone deacetylating activity of HDAC6 with an inhibition activity (IC₅₀) from about 0.000001 μM to about 0.001 μM.

43. The composition according to claim 37, wherein the HDAC inhibitor compound is selective toward HDAC6.

44-51. (canceled)

52. A method of treating a histone deacetylase (HDAC)-associated disease, comprising:
(a) providing at least one compound of Formula I according to claim 1; and
(b) administering a composition to a subject with symptoms of the HDAC-associated disease, comprising a therapeutic amount of the HDAC inhibitor compound and a pharmaceutically acceptable carrier, wherein the therapeutic amount is effective to inhibit the activity of at least one HDAC isoform and in treating the symptoms of the HDAC-associated disease,
wherein the therapeutic amount of the HDAC inhibitor compound is capable of achieving a half-maximal dose response (EC50) value of acetylated tubulin obtained in cell ranging between 0.05 μM to 0.5 μM,
wherein the HDAC-associated disease is characterized by lower level of acetylated tubulin in cells isolated from the subject with symptoms of the HDAC-associated disease relative to the level of acetylated tubulin in cells isolated from a healthy subject, and
wherein the HDAC-associated disease is selected from the group consisting of a cell proliferative disease, an autoimmune or inflammatory disorder, a neurodegenerative disease, or a combination thereof.

53-55. (canceled)

56. The method according to claim 52, wherein the HDAC inhibitor compound inhibits the histone deacetylating activity of at least one HDAC isoform with an inhibition activity (IC₅₀) of from about 0.005 μM to about 2.76 μM.

57. The method according to claim 52, wherein the HDAC inhibitor compound inhibits the histone deacetylating activity of HDAC6 with an inhibition activity (IC₅₀) from about 0.000001 μM to about 0.001 μM.

58. The method according to claim 57, wherein the HDAC inhibitor compound is selective toward HDAC6.

59-60. (canceled)

61. The method according to claim 58, wherein a ratio of the half-maximal dose response (EC50) value of acetylated histone obtained in cell with the HDAC inhibitor compound to the half-maximal dose response (EC50) value of acetylated tubulin obtained in cell with the HDAC inhibitor compound (in cell selectivity value) has a value of at least 2.0.

62. The method according to claim 58, wherein a ratio of the half-maximal dose response (EC50) value of acetylated histone obtained in cell with the HDAC inhibitor compound to the half-maximal dose response (EC50) value of acetylated tubulin obtained in cell with the HDAC inhibitor compound (in cell selectivity value) has a value of at least 50.0.

63-66. (canceled)

67. The method according to claim 52, wherein the cell proliferative disease is a cancer, selected from the group consisting of an ovarian cancer, a prostate cancer, a lung cancer, an acute myeloid leukemia, a multiple myeloma, a bladder carcinoma, a renal carcinoma, abreast carcinoma, a colorectal carcinoma, a neuroblastoma, a melanoma, a gastric cancer, or a combination thereof.

68. The method according to claim 52, wherein the autoimmune or inflammatory disorder is selected from the group consisting of a rheumatoid arthritis, a psoriasis, an inflammatory bowel disease, a multiple sclerosis, a systemic lupus erthematosus, an airway hyperresponsiveness, a Crohn's disease, an ulcerative colitis, or a combination thereof.

69. The method according to claim 52, wherein the neurodegenerative disorder is selected from the group consisting of a cerebral ischemia, a Huntington's disease, an amyotrophic lateral sclerosis, a spinal musclular atrophy, a Parkinson's disease, an Alzheimer's disease, or a combination thereof.
